(19) United States
(12) Patent Application Publication
(10) Pub. No.: US 2004/0043438 A1
(43) Pub. Date: Mar. 4, 2004

(54) Allergen mutants

(75) Inventors: Jens Holm, Copenhagen N (DK); Mercedes Ferreras, Copenhagen O (DK)

Correspondence Address: DARBY & DARBY P.C., P. O. BOX 5257, NEW YORK, NY, 10150-5257, US

(73) Assignee: ALK-Abello A/S, Horsholm (DK)

(21) Appl. No.: 10/440,516

(22) Filed: May 16, 2003

Related U.S. Application Data
(60) Provisional application No. 60/381,440, filed on May 16, 2002.

(51) Int. Cl. G01N 33/53; G01N 33/537; G01N 33/543; C07K 14/415; C07K 16/00

(52) U.S. Cl. 435/7.92; 530/370; 530/387.1

(57) ABSTRACT

Novel recombinant allergens with multiple mutations and reduced IgE binding affinity are disclosed. The allergens are mutants of naturally occurring allergens. The overall α-carbon backbone tertiary structure is essentially preserved. Also disclosed is a method for preparing such recombinant allergens as well as uses thereof.

### CROSS REFERENCE TO RELATED APPLICATIONS

The application claims benefit of priority under 35 U.S.C § 119(e) of provisional application serial No. 60/381,440, filed May 16, 2002, which is hereby incorporated by reference herein in its entirety.

### FIELD OF THE INVENTION

The present invention relates to diagnosis and treatment of allergy. More specifically the invention provides ways of obtaining mutated allergen molecules suitable for these purposes. The invention furthermore relates to novel recombinant allergens, which are mutants of naturally occurring allergens as well as their use. Also, the invention relates to a composition comprising a mixture of novel recombinant mutant allergens. Further, the invention relates to a method of preparing such recombinant mutant allergens as well as to pharmaceutical compositions, including vaccines, comprising the recombinant mutant allergens. In further embodiments, the present invention relates to methods of generating immune responses in a subject, vaccination or treatment of a subject as well as processes for preparing the compositions of the invention.

### BACKGROUND OF THE INVENTION

Genetically predisposed individuals become sensitised (allergic) to antigens originating from a variety of environmental sources, to the allergens of which the individuals are exposed. The allergic reaction occurs when a previously sensitised individual is re-exposed to the same or a homologous allergen. Allergic responses range from hay fever, rhinoconductivitis, rhinitis and asthma to systemic anaphylaxis and death in response to e.g. bee or hornet sting or insect bite. The reaction is immediate and can be caused by a variety of atopic allergens such as compounds originating from grasses, trees, weeds, insects, food, drugs, chemicals and perfumes.

However, the responses do not occur when an individual is exposed to an allergen for the first time. The initial adaptive response takes time and does usually not cause any symptoms. But when antibodies and T cells capable of reacting with the allergen have been produced, any subsequent exposure may provoke symptoms. Thus, allergic responses demonstrate that the immune response itself can cause significant pathological states, which may be life threatening.

The antibodies involved in atopic allergy belong primarily to immunoglobulins of the IgE class. IgE binds to specific receptors on the surface of mast cells and basophils. Following complex formation of a specific allergen with IgE bound to mast cells, receptor cross-linking on the cell surface results in signalling through the receptors and the physiological response of the target cells. Degranulation of a mast cell results in the release of i.a. histamine, heparin, a chemotactic factor for eosinophilic leukocytes, leukotrienes C4, D4 and E4, which cause prolonged constriction of the bronchial smooth muscle cells. The resulting effects may be systemic or local in nature.

The antibody-mediated hypersensitivity reactions can be divided into four classes, namely type I, type II, type III and type IV. Type I allergic reactions is the classic immediate hypersensitivity reaction occurring within seconds or minutes following antigen exposure. These symptoms are mediated by allergen specific IgE.

Commonly, allergic reactions are observed as a response to protein allergens present e.g. in pollens, house dust mites, animal hair and dandruff, venoms, and food products.

In order to reduce or eliminate allergic reactions, carefully controlled and repeated administration of allergy vaccines is commonly used. Allergy vaccination is traditionally performed by parenteral, intranasal, or sublingual administration in increasing doses over a fairly long period of time, and results in desensitisation of the patient. The exact immunological mechanism is not known, but induced differences in the phenotype of allergen specific T cells is thought to be of particular importance.

Allergy Vaccination

The concept of vaccination is based on two fundamental characteristics of the immune system, namely specificity and memory. Vaccination will prime the immune system of the recipient, and upon repeated exposure to similar proteins the immune system will be in a position to respond more rigorously to the challenge of for example a microbial infection. Vaccines are mixtures of proteins intended to be used in vaccination for the purpose of generating such a protective immune response in the recipient. The protection will comprise only components present in the vaccine and homologous antigens.

Compared to other types of vaccination allergy vaccination is complicated by the existence of an ongoing immune response in allergic patients. This immune response is characterised by the presence of allergen specific IgE mediating the release of allergic symptoms upon exposure to allergens. Thus, allergy vaccination using allergens from natural sources has an inherent risk of side effects being in the utmost consequence life threatening to the patient.

Approaches to circumvent this problem may be divided in three categories. In practise measures from more than one category are often combined. First category of measures includes the administration of several small doses over prolonged time to reach a substantial accumulated dose. Second category of measures includes physical modification of the allergens by incorporation of the allergens into gel substances such as aluminium hydroxide. Aluminium hydroxide formulation has an adjuvant effect and a depot effect of slow allergen release reducing the tissue concentration of active allergen components. Third category of measures include chemical modification of the allergens for the purpose of reducing allergenicity, i.e. IgE binding.

The detailed mechanism behind successful allergy vaccination remains controversial. It is, however, agreed that T cells play a key role in the overall regulation of immune responses. According to current consensus the relation between two extremes of T cell phenotypes, Th1 and Th2, determine the allergic status of an individual. Upon stimulation with allergen Th1 cells secrete interleukines dominated by interferon-y leading to protective immunity and the individual is healthy. Th2 cells on the other hand secrete predominantly interleukin 4 and 5 leading to IgE synthesis and eosinophilia and the individual is allergic. In vitro studies have indicated the possibility of altering the responses of allergen specific T cells by challenge with allergen derived peptides containing relevant T cell epitopes. Current approaches to new allergy vaccines are therefore largely based on addressing the T cells, the aim being to silence the T cells (anergy induction) or to shift the response from the Th2 phenotype to the Th1 phenotype.

Antibody-Binding Epitopes (B-Cell Epitopes)

X-ray crystallographic analyses of F_{ab}-antigen complexes has increased the understanding of antibody-binding epitopes. According to this type of analysis antibody-binding epitopes can be defined as a section of the surface of the antigen comprising atoms from 15-25 amino acid residues, which are within a distance from the atoms of the antibody enabling direct interaction. The affinity of the antigen-antibody interaction can not be predicted from the enthalpy contributed by van der Waals interactions, hydrogen bonds or ionic bonds, alone. The entropy associated with the almost complete expulsion of water molecules from the interface represent an energy contribution similar in size. This means that perfect fit between the contours of the interacting molecules is a principal factor underlying antigen-antibody high affinity interactions.

In WO 97/30150 (ref. 1), a population of protein molecules is claimed, which protein molecules have a distribution of specific mutations in the amino acid sequence as compared to a parent protein. From the description, it appears that the invention is concerned with producing analogues which are modified as compared to the parent protein, but which are taken up, digested and presented to T cells in the same manner as the parent protein (naturally occurring allergens). Thereby, a modified T cell response is obtained. Libraries of modified proteins are prepared using a technique denoted PM (Parsimonious Mutagenesis).

In WO 92/02621 (ref. 2), recombinant DNA molecules are described, which molecules comprise a DNA coding for a polypeptide having at least one epitope of an allergen of trees of the order Fagales, the allergen being selected from Aln g 1, Cor a 1 and Bet v 1. The recombinant molecules described herein do all have an amino acid sequence or part of an amino acid sequence that corresponds to the sequence of a naturally occurring allergen.

WO 90/11293 (ref. 3) relates i.a. to isolated allergenic peptides of ragweed pollen and to modified ragweed pollen peptides. The peptides disclosed therein have an amino acid sequence corresponding either to the sequence of the naturally occurring allergen or to naturally occurring isoforms thereof.

Chemical Modification of Allergens

Several approaches to chemical modification of allergens have been taken. Approaches of the early seventies include chemical coupling of allergens to polymers, and chemical cross-linking of allergens using formaldehyde, etc., producing the so-called ‘allergoids’. The rationale behind these approaches was random destruction of IgE binding epitopes by attachment of the chemical ligand thereby reducing IgE-binding while retaining immunogenicity by the increased molecular weight of the complexes. Inherent disadvantages of ‘allergoid’ production are linked to difficulties in controlling the process of chemical cross-linking and difficulties in analysis and standardisation of the resulting high molecular weight complexes. ‘Allergoids’ are currently in clinical use and due to the random destruction of IgE binding epitopes higher doses can be administered as compared to conventional vaccines, but the safety and efficacy parameters are not improved over use of conventional vaccines.

More recent approaches to chemical modification of allergens aim at a total disruption of the tertiary structure of the allergen thus eliminating IgE binding assuming that the essential therapeutic target is the allergen specific T cell. Such vaccines contain allergen sequence derived synthetic peptides representing minimal T cells epitopes, longer peptides representing linked T cells epitopes, longer allergen sequence derived synthetic peptides representing regions of immunodominant T cell epitopes, or allergen molecules cut in two halves by recombinant technique. Another approach based on this rationale has been the proposal of the use of “low IgE binding” recombinant isoforms. In recent years it has become clear that natural allergens are heterogeneous containing isoallergens and variants having up to approximately 25% of their amino acids substituted. Some recombinant isoallergens have been found to be less efficient in IgE binding possibly due to irreversible denaturation and hence total disruption of tertiary structure.

In Vitro Mutagenesis and Allergy Vaccination

Attempts to reduce allergenicity by in vitro site directed mutagenesis have been performed using several allergens including Der f 2 (Takai et al, ref. 4), Der p 2 (Smith et al, ref. 5), a 39 kDa Dermatophagoides farinae allergen (Aki et al, ref. 6), bee venom phospholipase A2 (Förster et al, ref. 7), Ara h 1 (Burks et al, ref. 8), Ara h 2 (Stanley et al, ref. 9), Bet v 1 (Ferreira et al, ref. 10 and 11), birch profilin (Wiedemann et al, ref. 12), and Ory s 1 (Alvarez et al, ref. 13).

The rationale behind these approaches, again, is addressing allergen specific T cells while at the same time reducing the risk of IgE mediated side effects by reduction or elimination of IgE binding by disruption of the tertiary structure of the recombinant mutant allergen.

The article by Ferreira et al (ref. 11) discloses the use of site directed mutagenesis for the purpose of reducing IgE binding. Although the three-dimensional structure of Bet v 1 is mentioned in the article the authors do not use the structure for prediction of solvent exposed amino acid residues for mutation, half of which have a low degree of solvent exposure. Rather they use a method developed for prediction of functional residues in proteins. Although the authors do discuss conservation of α-carbon backbone tertiary structure this concept is not a part of the therapeutic strategy but merely included to assess in vitro IgE binding. Furthermore, the evidence presented is not adequate since normalisation of CD-spectra prevents the evaluation of denaturation of a proportion of the sample, which is a common problem. The therapeutic strategy described aim at inducing tolerance in allergen specific T cells and initiation of a new immune response is not mentioned.

The article by Wiedemann et al. (ref. 12) describes the use of site directed mutagenesis and peptide synthesis for the purpose of monoclonal antibody epitope characterisation. The study demonstrates that substitution of a surface exposed amino acid has the capacity to modify the binding characteristics of a monoclonal antibody, which is not surprising considering common knowledge. The experiments described are not designed to assess modulation in the binding of polyclonal antibodies such as allergic patients' serum IgE. One of the experiments does apply serum IgE and although this experiment is not suitable for quantitative assessment, IgE binding does not seem to be affected by the mutations performed.

The article by Smith et al. (ref. 5) describes the use of site directed mutagenesis for the purpose of monoclonal antibody epitope mapping and reduction of IgE binding. The authors have no knowledge of the tertiary structure and make no attempt to assess the conservation of α-carbon backbone tertiary structure. The algorithm used does not ensure that amino acids selected for mutation are actually exposed to the molecular surface. Only one of the mutants described lead to a substantial reduction in IgE binding. This mutant is deficient in binding of all antibodies tested indicating that the tertiary structure is disrupted. The authors do not define a therapeutic strategy and initiation of a new immune response is not mentioned.

The article by Colombo et al. (ref. 14) describes the study of an IgE binding epitope by use of site directed mutagenesis and peptide synthesis. The authors use a three dimensional computer model structure based on the crystal structure of a homologous protein to illustrate the presence of the epitope on the molecular surface. The further presence of an epitope on a different allergen showing primary structure homology is addressed using synthetic peptides representing the epitope. The therapeutic strategy is based on treatment using this synthetic peptide representing a monovalent IgE binding epitope.

The article by Spangfort et al. (ref. 15) describes the three-dimensional structure and conserved surface exposed areas of the major birch allergen. The article does not disclose site directed mutagenesis, neither is therapeutic application addressed.

In none of the studies described above is IgE binding being reduced by substitution of surface exposed amino acids while conserving α-carbon backbone tertiary structure. Neither is the concept of initiating a new protective immune response mentioned.

WO 01/83559 discloses a method of selecting a protein variant with modified immunogenicity by using antibody binding peptide sequences to localise epitope sequences on the 3-dimensional structure of the parent protein. An epitope area is subsequently defined and one or more of the amino acids defining the epitope area are mutated. The invention is exemplified by industrial enzymes that function as allergens.

WO 99/47680 discloses the introduction of artificial amino acid substitutions into defined critical positions while retaining the α-carbon backbone tertiary structure of the allergen. In particular, WO 99/47680 discloses a recombinant allergen, which is a non-naturally occurring mutant derived from a naturally occurring allergen, wherein at least one surface-exposed, conserved amino acid residue of a B cell epitope is substituted by another residue which does not occur in the same position in the amino acid sequence of any known homologous protein within the taxonomic order from which said naturally occurring allergen originates, said mutant allergen having essentially the same α-carbon backbone tertiary structure as said naturally occurring allergen, and the specific IgE binding to the mutated allergen being reduced as compared to the binding to said naturally occurring allergen.

The recombinant allergen disclosed in WO 99/47680 is obtainable by a) identifying amino acid residues in a naturally occurring allergen which are conserved with more than 70% identity in all known homologous proteins within the taxonomic order from which said naturally occurring allergen originates, b) defining at least one patch of conserved amino acid residues being coherently connected over at least 400 Å² of the surface of the three-dimensional structure of the allergen molecule as defined by having a solvent accessibility of at least 20%, said at least one patch comprising at least one B cell epitope, and c) substituting at least one amino acid residue in said at least one patch by another amino acid being non-conservative in the particular position while essentially preserving the overall α-carbon backbone tertiary structure of the allergen molecule.

Patent application PCT/DK 01/00764 relates to mutants of naturally occurring allergens. The following specific Bet v 1 mutants are disclosed therein:

Mutant A: Asn28Thr, Lys32Gln, Asn78Lys, Lys103Val, Arg145Glu, Asp156His, +160Asn.

Mutant B: Tyr5Val, Glu42Ser, Glu45Ser, Asn78Lys, Lys103Val, Lys123lle, Lys134Glu, Asp156His.

Mutant 2628: Tyr5Val, Glu45Ser, Lys65Asn, Lys97Ser, Lys134Glu.

Mutant 2637: Ala16Pro, Asn28Thr, Lys32Gln, Lys103Thr, Pro108Gly, Leu152Lys, Ala153Gly, Ser155Pro.

Mutant 2724: N28T, K32Q, N78K, K103V, P108G, R145E, D156H, +160N.

Mutant 2733: Tyr5Val, Lys134Glu, Asn28Thr, Lys32Gln, Glu45Ser, Lys65Asn, Asn78Lys, Lys103Val, Lys97Ser, Pro108Gly, Arg145Glu, Asp156His, +160Asn.

Mutant 2744: Tyr5Val, Lys134Glu, Glu42Ser, Glu45Ser, Asn78Lys, Lys103Val, Lys123lle, Asp156His, +160Asn.

Mutant 2753: Asn28Thr, Lys32Gln, Lys65Asn, Glu96Leu, Lys97Ser, Pro108Gly, Asp109Asn, Asp125Tyr, Glu127Ser, Arg145Glu.

Mutant 2744+2595: Y5V, N28T, K32Q, E42S, E45S, N78K, K103V, P108G, K123I, K134E, D156H, +160N.

Mutant 2744+2628: Y5V, E42S, E45S, K65N, N78K, K97S, K103V, K123I, K134E, D156H, +160N.

Mutant 2744+2595+2628: Y5V, N28T, K32Q, E42S, E45S, K65N, N78K, K97S, K103V, P108G, K123I, K134E, D156H, +160N.

### BRIEF DESCRIPTION OF THE FIGURES

FIG. 1 shows a theoretical model of the reaction between an allergen and mast cells by IgE cross-linking.

FIG. 2. (Left) The molecular surface of Bet v 1 with the location of group 1 to 10 shown in black and grey tones. (Right) View of the amino acid residues constituting group 1 to 10. Groups are marked 1 to 10.

FIG. 3 shows mutant-specific oligonucleotide primers used for mutation of Bet v 1 mutant number 1. Mutated nucleotides are underlined.

FIG. 4 shows oligonucleotide primers for site directed mutagenesis of Bet v 1 (No. 2801). Two generally applicable primers denoted “all-sense” and “all non-sense” were used for all mutants.

FIG. 5 shows the DNA and amino acid sequence of the naturally occurring allergen Bet v 1 as well as a number of Bet v 1 mutations.

FIG. 6 shows the inhibition of the binding of biotinylated recombinant Bet v 1 to serum IgE from a pool of allergic patients by non-biotinylated Bet v 1 and by Bet v 1 Glu45Ser mutant.

FIG. 7 shows the inhibition of the binding of biotinylated recombinant Bet v 1 to serum IgE from a pool of allergic patients by non-biotinylated Bet v 1 and by Bet v 1 mutant Asn28Thr+Lys32Gln.

FIG. 8 shows the inhibition of the binding of biotinylated recombinant Bet v 1 to serum IgE from a pool of allergic patients by non-biotinylated Bet v 1 and by Bet v 1 Pro108Gly mutant.

FIG. 9 shows the inhibition of the binding of biotinylated recombinant Bet v 1 to serum IgE from a pool of allergic patients by non-biotinylated Bet v 1 and by Bet v 1 Glu60Ser mutant.

FIG. 10 shows the CD spectra of recombinant and the (Asn28Thr, Lys32Gln, Glu45Ser, Pro108Gly) mutant, recorded at close to equal concentrations.

FIG. 11 shows the inhibition of the binding of biotinylated recombinant Bet v 1 to serum IgE from a pool of allergic patients by non-biotinylated Bet v 1 and by the (Asn28Thr, Lys32Gln, Glu45Ser, Pro108Gly) mutant.

FIG. 12 shows a graphical illustration of the 2-step PCR mutation technique used for generating mutated Bet v 1 allergens. (I) shows the sense and antisense primers. (II) shows the final recombinant protein harbouring mutations at the indicated positions. Lines represent DNA sequences; numbers in parentheses above lines represent sense oligonucleotide primers (1), (3), (5), (7), (9) and (11); numbers in parentheses below lines represent anti-sense nucleotide primers (2), (4) (6), (8), (10) and (12); notation is X (position) Y represents mutations; (1) represents the sense oligonucleotide primer accommodating the proteins N-terminus; and (12) represents the anti-sense oligonucleotide accommodating the protein C-terminus.

FIG. 13 shows a graphical illustration of the PCR mutation events leading to the cloning of Bet v 1 (3004), (3005), (3007) and (3009). Primers used for introducing point mutations are listed.

FIG. 14 shows a graphical illustration of the PCR mutation events leading to the cloning of Bet v 1 (3031) to (3045). Degenerated primers used for introducing random mutations in position 10, 20, 36, 73, 87, 129 and 149 are listed. The possible outcome of mutation for each position is shown at the top.

FIG. 15 shows schematically the primers used to create the mutations. (I) shows the sense and antisense primers. (II) shows the final recombinant protein harbouring mutations at the indicated positions. Lines represent DNA sequences; numbers in parentheses above lines represent sense oligonucleotide primers (1), (3), (5), (7), (9) and (11); numbers in parentheses below lines represent anti-sense nucleotide primers (2), (4) (6), (8), (10) and (12); notation is X (position) Y represents mutations; (1) represents the sense oligonucleotide primer accommodating the proteins N-terminus; and (12) represents the anti-sense oligonucleotide accommodating the protein C-terminus.

FIG. 16 shows an illustration of the construction of Bet v 1 mutants and a listing of the primers used. The mutants contain from five to nine amino acids.

FIG. 17 shows introduced point mutations at the surface of Bet v 1 (2628) and Bet v 1 (2637) showing backbone+amino acids 95-100% conserved among Fagales (grey) and introduced point mutations (black). In mutant Bet v 1 (2628), five primary mutations were introduced in one half of Bet v 1 leaving the other half unaltered. In mutant Bet v 1 (2637), five primary and three secondary mutations were introduced in the other half, leaving the first half unaltered.

FIG. 18 shows the circular dichroism (CD) spectra of recombinant Bet v 1.2801 (wild type; closed circles) and the Bet v 1 (2637) mutant (open squares) recorded at nearly identical concentrations.

FIG. 19 shows the inhibition of the binding of biotinylated recombinant Bet v 1.2801 (wild type) to serum IgE from a pool of allergic patients by non-biotinylated Bet v 1.2801 and by Bet v 1 (2628), Bet v 1 (2637), and a 1:1 mix of Bet v 1 (2628) and Bet v1 (2637).

FIG. 20 shows histamine release in human basophil cells of Bet v 1.2801 (wild type), Bet v 1 (2628), and Bet v 1 (2637).

FIG. 21 shows histamine release in human basophil cells of Bet v 1.2801 (wild type), Bet v 1 (2628), and Bet v 1 (2637).

FIG. 22 shows point mutations at the surface of Bet v 1 (2744). Grey: Back bone+Amino acid residues 95-100% conserved among Fagales. Black: Point mutations.

FIG. 23 shows point mutations at the surface of Bet v 1 (2753). Grey: Back bone+Amino acid residues 95-100% conserved among Fagales. Black: Point mutations.

FIG. 24 shows point mutations at the surface of Bet v 1 (2744) [white] and Bet v 1 (2753) [Black]. Grey: Back bone+Amino acid residues 95-100% conserved among Fagales. Black: Mutations (Y5V, K134E), (E42S, E45S), (N78K, K103V), K123 I, (D156H, +160N). White: Mutations (N28T, K32Q), K65N, (E96L, K97S), (P108G, D109N), (D125Y, E127S), R145E.

FIG. 25 shows circular dichroism (CD) spectra of Bet v 1.2801 (wild type) and Bet v 1 (2744), recorded at nearly equal concentrations.

FIG. 26 shows histamine release in human basophil cells of Bet v 1.2801 (wild type), and mutant Bet v 1 (2744).

FIGS. 27A-D shows histamine release in human basophil cells of Bet v 1.2801 (wild type), and mutant Bet v 1 (2744). (A) Donor MJ (B) Donor MH (C) Donor CJB (D) Donor MCDS.

FIG. 28 shows point mutations at the surface of Bet v 1 (2733). Grey: Back bone+Amino acid residues 95-100% conserved among Fagales. Black: Point mutations: Y5V, N28T, K32Q, E45S, K65N, N78K, K97S, K103V, P108G, K134E, R145E, D156H, +160N

FIG. 29 shows the proliferation of patient Peripheral Blood Lymphocytes expressed as Stimulation Index (SI) for various Bet v 1 preparations.

FIG. 30 shows the cytokine profile of T cells stimulated with various Bet v 1 preparations from a patient with a Th0 profile.

FIG. 31 shows the cytokine profile of T cells stimulated with various Bet v 1 preparations from a patient with a Th1 profile.

FIG. 32 shows the cytokine profile of T cells stimulated with various Bet v 1 preparations from a patient with a Th2 profile.

FIG. 33 shows Circular dichroism (CD) spectroscopy of rBet v 1.2801(●) (wildtype) and the rBet v 1 3007) mutant [Δ] with 12 mutations, recorded at equal concentrations. Overlay of circular dichroism (CD) spectra obtained at 15° C. are shown.

FIG. 34 shows the inhibition of the binding of biotinylated rBet v 1.2801 to pooled IgE serum from birch allergic patients by rBet v 1.2801 (●) (wildtype) or mutated rBet v 1 (3007) [Δ] with 12 mutations.

### OBJECT OF THE INVENTION

The object of the invention is to provide improved recombinant mutant allergen proteins.

Rationale Behind the Present Invention

The current invention is based on a unique rationale. According to this rationale the mechanism of successful allergy vaccination is not an alteration of the ongoing Th2-type immune response, but rather a parallel initiation of a new immune response involving tertiary epitope recognition by B-cells and antibody formation. It is believed that this new immune response is partly a Th1-type immune response. When the vaccine (or pharmaceutical compositions) is administered through another route than the airways, it is hypothesised, that the new immune response evolves in a location physically separated from the ongoing Th2 response thereby enabling the two responses to exist in parallel.

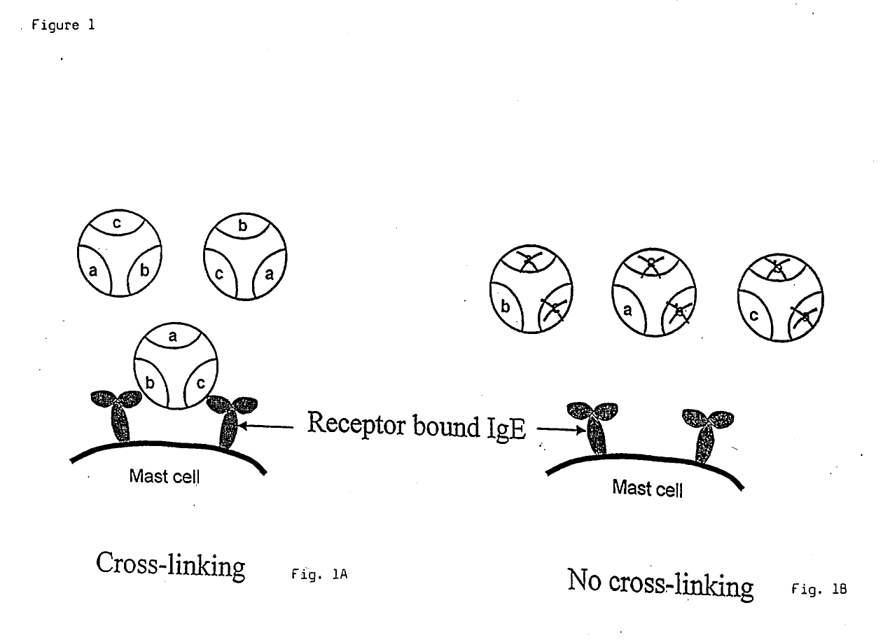

Furthermore, the invention is based on the finding that allergic symptoms are triggered by the cross-linking of allergen with at least two specific IgE's bound to the surface of effector cells, i.e. mast cells and basophils, via the high affinity IgE receptor, FcεRI. For illustration, we refer to FIG. 1, which depicts a theoretical model of an allergen with three IgE binding epitopes. Induction of mediator release from the mast cell and hence allergic symptoms is effected by allergen-mediated cross-linking of IgE bound to the surface of the mast cell, cf. FIG. 1A. In the situation shown in FIG. 1B two of the epitopes have been mutated so as to reduce their IgE binding ability, and hence the allergen-mediated cross-linking is prevented. In this connection it should be noted that allergens usually comprise more than three B cell epitopes.

In order for a mutant allergen to be able to raise the new immune response, including an IgG response, the mutant allergen must comprise at least one intact epitope or an epitope, which has been altered only moderately. The surface topography of a moderately altered epitope preferably resembles the original epitope, allowing new more numerous IgG antibodies to be raised. These new IgG antibodies have specificities which can compete and to some degree oust IgE binding to the natural occurring allergen. Further, it may be assumed that the more epitopes, which have been mutated so as to eliminate or reduce their IgE binding ability, the lower the risk of allergen-mediated cross-linking and resulting allergic symptoms upon administration of an allergen vaccine.

According to this rationale it is essential that the mutant allergen has an α- carbon backbone tertiary structure which is essentially the same as that of the natural allergen.

It has previously been assumed that positions suitable for mutation are located exclusively in areas consisting of conserved amino acid residues believed to harbour dominant IgE binding epitope. However, according to the present invention it appears that surface exposed amino acid residues suitable for mutation comprise both highly conserved residues and residues that are not conserved or only conserved to a smaller degree. Such amino acid residues appear to be distributed over the entire molecular surface with a tendency to form small groupings covering a defined area on the molecular surface.

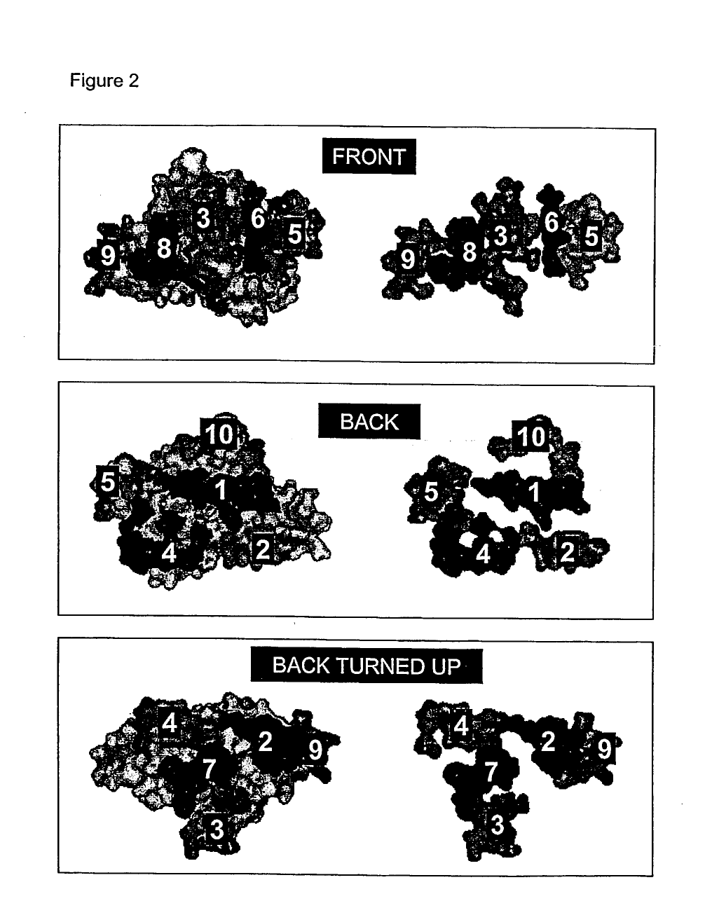

Thus, according to the present invention, surface exposed amino acids suitable for mutation can be divided into groups as illustrated in FIG. 2. The groupings rely on the tendency of these amino acid residues to form separate areas and these groupings are furthermore independent of the degree of conservation of the amino acid residues. Each group represents a number of surface exposed amino acid residues that are found within a limited area on the surface of the allergen. Each individual group most likely comprises part of at least one epitope or at least one intact epitope. Each separate group may comprise as well amino acids positions that will result in a moderately altered epitope upon mutation as well as amino acid positions that will result in a more drastic alteration of the epitope upon mutation. A single amino acid residue typically results in a moderate alteration of an epitope if the original amino acid residue is substituted with an amino acid that posseses similar chemical features (E.g. exchanging a hydrophobic amino acid with another hydrophobic amino acid residue). In conclusion, by selecting mutations among amino acid residues from at least four of the defined groups provides a tool for rendering it very likely that a mutant allergen according to the present invention is mutated in several B-cell epitopes and has a α-carbon backbone structure that is similar to the naturally occurring allergen.

It is furthermore an important aspect of the present invention that the mutated allergen retains a continous surface region with an area of about 400-800 Å² that contains either no mutations or only moderate mutations. It is believed that an allergen comprises a number of potential binding regions for specific IgE's, wherein each region has an area of approximately 800 Å².

The inventive idea of the present invention is based on the recognition that a mutated allergen having IgE binding reducing mutations in at least 4 defined groups, each group comprising surface exposed amino acids suitable for mutation, but retaining at least one intact or moderately altered epitope, would on the one hand reduce the allergen-mediated cross-linking and on the other hand allow the raising of an IgG response with a binding ability competitive with that of IgE. Thus, the said mutated allergen constitutes a highly advantageous allergen in that the risk of anaphylactic reactions is being strongly reduced. The mutant allergen has the potential to be administered in relatively higher doses improving its efficacy in generating a protective immune response without compromising safety.

Also, the present invention is based on the recognition that a vaccine comprising a mixture of different such mutated allergens, wherein ideally many or all epitopes are represented as intact epitopes or epitopes that are only moderately altered on different mutated allergens, would be equally efficient in its ability to induce protection against allergic symptoms as the natural occurring allergen from which the mutated allergens are derived.

### SUMMARY OF THE INVENTION

The present invention relates to the introduction of amino acid substitutions into allergens. The amino acid substitutions are chosen from at least four groups of amino acids suitable for amino acid substitution. The object being to reduce the specific IgE binding capability of each mutated epitope while retaining at least one intact or only moderately altered epitope on the mutated allergen.

In particular the present invention relates to a recombinant Bet v 1 allergen, characterised in that it is a mutant of a naturally occurring Bet v 1 allergen where:

a. the mutant retains essentially the same α-carbon backbone structure as the naturally occurring allergen,

b. the mutant comprises at least four primary mutations, which each reduce the specific IgE binding capability of the mutated allergen as compared to the IgE binding capability of the naturally occurring Bet v 1 allergen,

c. each primary mutation is a substitution of one surface-exposed amino acid residue with another residue,

d. the mutations are placed in such a manner that at least one area of 400-800 Å² comprises either no mutations or one or more moderate mutations,

e. the primary mutations are selected from at least 4 of the following 10 groups, each group comprising surface exposed amino acid positions suitable for amino acid substitution:

group 1: A130, E131, K134, A135, K137, E138, E141, T142, R145;

group 2: V2, F3, N4, Y5, E6, T7, K 119;

group 3: D27, S39, S40, Y41, E42, N43, 144, E45, G46, N47, P50, G51, K55, D72, E73;

group 4: E8, T10, V12, P14, V105, A106, T107, P108, D109, G110, I113, K115;

group 5: A16, K20, S149, Y150, L152, A153, H154, S155, D156, Y158, N159, +160, wherein +160 represents addition of an N-terminal amino acid;

group 6: L24, D25, N28, K32;

group 7: H76, T77, N78, F79, K80, E101, K103;

group 8: K68, R70, I86, E87, E96, K97;

group 9: G1, G92, D93, T94, K123, G124, D125, H126, E127, K129;

group 10: P35, Q36, E60, G61, P63, F64, K65, Y66;

with the proviso that the recombinant Bet v 1 allergen is not one of the following specific mutants: (Asn28Thr, Lys32Gln, Asn78Lys, Lys103Val, Arg145Glu, Asp156His, +160Asn); (Tyr5Val, Glu42Ser, Glu45Ser, Asn78Lys, Lys103Val, Lys123lle, Lys134Glu, Asp156His); (Tyr5Val, Glu45Ser, Lys65Asn, Lys97Ser, Lys134Glu); (Ala16Pro, Asn28Thr, Lys32Gln, Lys103Thr, Pro108Gly, Leu152Lys, Ala153Gly, Ser155Pro); (N28T, K32Q, N78K, K103V, P108G, R145E, D156H, +160N); (Tyr5Val, Lys134Glu, Asn28Thr, Lys32Gln, Glu45Ser, Lys65Asn, Asn78Lys, Lys103Val, Lys97Ser, Pro108Gly, Arg145Glu, Asp156His, +160Asn); (Tyr5Val, Lys134Glu, Glu42Ser, Glu45Ser, Asn78Lys, Lys103Val, Lys231lle, Asp156His, +160Asn); (Asn28Thr, Lys32Gln, Lys65Asn, Glu96Leu, Lys97Ser, Pro108Gly, Asp109Asn, Asp125Tyr, Glu127Ser, Arg145Glu); (Y5V, N28T, K32Q, E42S, E45S, N78K, K103V, P108G, K123I, K134E, D156H, +160N); (Y5V, E42S, E45S, K65N, N78K, K97S, K103V, K123I, K134E, D156H, +160N); and (Y5V, N28T, K32Q, E42S, E45S, K65N, N78K, K97S, K103V, P108G, K1231, K134E, D156H, +160N).

More specifically, the present invention relates to a recombinant Bet v 1 allergen where the primary mutations are selected from at least 4 of the following 10 groups, each group comprising surface exposed amino acid positions suitable for the following amino acid substitutions:

group 1: A130: A130V, A130G, A130I, A130L, A130S, A130H, A130T; E131: E131D, E131H, E131K, E131R, E131S; K134: K134R, K134H, K134S, K134Q, K1341, K134E; A135: A135V, A135G, A135I, A135L, A135S, A135H, A135T; K137: K137R, K137H, K137S, K137Q, K1371, K137E; E138: E138D, E138H, E138K, E138R, E138S, E138N; E141: E141D, E141H, E141K, E141R, E141S; T142: T142A, T142S, T142L, T142V, T142D, T142K, T142N; R145: R145K, R145H, R145T, R145D, R145E;

group 2: V2: V2A, V21, V2K, V2L, V2R, V2T; F3: F3H, F3W, F3S, F3D N4: N4H, N4K, N4M, N4Q, N4R; Y5: Y5D, Y5G, Y5H, Y51, Y5K, Y5V; E6: E6D, E6H, E6K, E6R, E6S; T7: T7P, T7S, T7L, T7V, T7D, T7K, T7N; K119: K119R, K119H, K119S, K119Q, K1191, K119E, K119N;

group 3: D27: D27E, D27H, D27K, D27R, D27S; S39: S39T, S39L, S39V, S39D, S39K; S40: S40T, S40L, S40V, S40D, S40K; Y41: Y41D, Y41G, Y41H, Y41I, Y41K, Y41V; E42: E42S, E42D, E42H, E42K, E42R; N43: N43H, N43K, N43M, N43Q, N43R; I44: I44L, I44K, I44R, I44D; E45: E45S, E45D, E45H, E45K, E45R; G46: G46N, G46H, G46K, G46M, G46Q, G46R; N47: N47H, N47K, N47M, N47Q, N47R; P50: P50G; G51: G51N, G51H, G51K, G51M, G51Q, G51R; K55: K55R, K55H, K55S, K55Q, K55I, K55E, K55N; D72: D72E, D72S, D72H, D72R, D72K; E73: E73D, E73S, E73H, E73R, E73K;

group 4: E8: E8D, E8H, E8K, E8R, E8S; T10: T10P, T10S, T10L, T10V, T10D, T10K, T10N; V12: V12A, V12I, V12K, V12L, V12R, V12T; P14: P14G; V105: V105A, V105I, V105K, V105L, V105R, V105T; A106: A106V, A106G, A106I, A106L, A106S, A106H, A106T; T107: T107A, T107S, T107L, T107V, T107D, T107K, T107N; P108: P108G; D109: D109N D109E, D109S, D109H, D109R, D109K; G110: G110N, G110H, G110K, G110M, G110Q, G110R; I113: I113L, I113K, I113R, I113D, K115: K115R, K115H, K115S, K115Q, K151, K115E, K115N;

group 5: A16: A16V, A16G, A16I, A16L, A16S, A16H, A16T; K20: K20R, K20H, K20S, K20Q, K20I, K20E, K20N; S149: S149T, S149L, S149V, S149D, S149K; Y150: Y150T, Y150L, Y150V, Y150D, Y150K; L152: L152A, L152V, L152G, L1521, L152S, L152H, L152T; A153: A153V, A153G, A153I, A153L, A153S, A153H, A153T; H154: H154W, H154F, H154S, H154D; S155: S155T, S155L, S155V, S155D, S155K; D156: D156H, D156E, D156S, D156R, D156K; Y158: Y158D, Y158G, Y158H, Y158I, Y158K, Y158V; N159: N159H, N159K, N159M, N159Q, N159R, N159G, +160N;

group 6: L24: L24A, L24V, L24G, L24I, L24S, L24H, L24T; D25: D25E, D25H, D25K, D25R, D25S; N28: N28H, N28K, N28M, N28Q, N28R, N28T; K32: K32Q, K32R, K32N, K32H, K32S, K321, K32E;

group 7: H76: H76W, H76F, H76S, H76D; T77: T77A, T77S, T77L, T77V, T77D, T77K, T77N; N78: N78H, N78K, N78M, N78Q, N78R; F79: F79H, F79W, F79S, F79D; K80: K80R, K80H, K80S, K80Q, K801, K80E, K80N; E101: E101D, E101H, E101K, E101R, E101S; K103: K103R, K103H, K103S, K103Q, K103I, K103E, K103V;

group 8: K68: K68R, K68H, K68S, K68Q, K681, K68E, K68N; R70: R70K, R70H, R70T, R70D, R70E, R70N; 186: 186L, I86K, I86R, I86D; E87: E87D, E87H, E87K, E87R, E87S, E87A; E96: E96D, E96H, E96K, E96R, E96S, E96L; K97: K97R, K97H, K97S, K97Q, K971, K97E;

group 9: G1: G1N, G1H, G1K, G1M, G1Q, G1R; G92: G92N, G92H, G92K, G92M, G92Q, G92R; D93: D93N, D93E, D93S, D93H, D93R, D93K; T94: T94A, T94S, T94L, T94V, T94D, T94K, T94N; K123: K123R, K123H, K123S, K123Q, K123I, K123E; G124: G124N, G124H, G124K, G124M, G124Q, G124R; D125: D125E, D125H, D125K, D125R, D125S, D125Y; H126: H126W, H126F, H126S, H126D; E127: E127D, E127H, E127K, E127R, E127S; K129: K129R, K129H, K129S, K129Q, K129I, K129E, K129N;

group 10: P35: P35G; Q36: Q36K, Q36R, Q36N, Q36H, Q36S, Q36I, Q36E; E60: E60H, E60K, E60M, E60Q, E60R; G61: G61N, G61H, G61K, G61M, G61Q, G61R; P63: P63G; F64: F64H, F64W, F64S, F64D; K65: K65R, K65H, K65S, K65Q, K651, K65E, K65N; Y66: Y66D, Y66G, Y66H, Y661, Y66K, Y66V.

The present invention further relates to a recombinant Bet v 1 mutant allergen comprising substitutions that are selected from at least four of the following 10 groups:

Group 1: A130V, K134E, E141N,

Group 2: V2L, Y5V, E6S, K119N,

Group 3: E42S, E45S, N47K, K55N, E73S, E73T, E73S,

Group 4: E8S, T10P, P14G, P108G, D109N, K115N,

Group 5: A16G, K20S, S149T L152A A153V, S155T, N159G, +160N,

Group 6: L24A, D25E, N28T, K32Q,

Group 7: T77A, T77N, N78K, K103V,

Group 8: R70N, E87A, E96S, K97S,

Group 9: D93S, K1231, D125Y, K129N,

Group 10: Q36N, E60S, G61S, P63G.

The present invention further relates to a recombinant Bet v 1 mutant allergen comprising substitutions that are selected from at least four of the following 10 groups:

Group 1: K134E,

Group 2: Y5V, K119N, V2L,

Group 3: E45S, E42S, K55N, N47K, E73S,

Group 4: E96S, K97S, P108G, D109N, T10N, K115N, P14G,

Group 5: N159G, +160N, S149T, A153V, L152A, A16G, K20S,

Group 6: N28T, K32Q, L24A,

Group 7: K103V, T77N, N78K,

Group 8: E96S, K97S, E87A,

Group 9: K129N, D125Y, K123I, D93S,

Group 10: E60S, Q36N, G61S, P63G.

The present invention further relates to the following:

Recombinant Bet v 1 allergens variants that can be used as a pharmaceutical and for preparing a pharmaceutical for preventing and/or treating birch pollen allergy.

A composition comprising two or more different recombinant mutant Bet v 1 allergen variants according to the present invention wherein each variant has at least one primary mutation, which is absent in at least one of the other variants. The composition comprises 2-12, preferably 3-10, more preferably 4-9 and most preferably 5-8 variants. A composition according to the present invention can be used as a pharmaceutical and for preparing a pharmaceutical for preventing and/or treating birch pollen allergy. The pharmaceutical composition preferably comprises a pharmaceutically acceptable carrier, and/or excipient, and optionally an adjuvant.

A pharmaceutical composition in the form of a vaccine against allergic reactions elicited by a naturally occurring Bet v 1 allergen in patients suffering from birch pollen allergy.

Methods of generating an immune response in a subject comprising administering to a subject a recombinant allergen, a composition, or a pharmaceutical composition.

Vaccination or treatment of a subject comprises administering to the subject a recombinant allergen, a composition, or a pharmaceutical composition.

A method for preparing a pharmaceutical composition comprising mixing a recombinant allergen, or a composition with pharmaceutically acceptable substances, and/or excipients.

A method for the treatment, prevention or alleviation of allergic reactions in a subject that comprises administering to a subject a recombinant Bet v 1 allergen, a composition, or a pharmaceutical composition.

A method of preparing a recombinant Bet v 1 allergen characterised in that the substitution of amino acids is carried out by site-directed mutagenesis, or DNA shuffling (molecular breeding) (Punnonen et al., ref. 25).

A DNA sequence encoding a recombinant Bet v 1 allergen, a derivative thereof, a partial sequence thereof, a degenerated sequence thereof or a sequence which hybridises thereto under stringent conditions, wherein said derivative, partial sequence, degenerated sequence or hybridising sequence encodes a peptide having at least one B cell epitope.

A DNA sequence which is a derivative of the DNA sequence encoding the naturally occurring allergen. The DNA sequence encoding the derivative is obtained by site-directed mutagenesis of the DNA encoding the naturally occurring Bet v 1 allergen.

An expression vector comprising DNA encoding a recombinant Bet v 1 variant, a host cell comprising the expression vector, and a method of producing a recombinant mutant Bet v 1 allergen comprising cultivating the host cell.

A recombinant Bet v 1 allergen or a recombinant Bet v 1 allergen that is encoded by the DNA sequence comprises at least one T celle epitope capable of stimulating a T cell clone or T cell line specific for the naturally occurring Bet v 1 allergen.

A diagnostic assay for assessing relevance, safety, or outcome of therapy of a subject using a recombinant mutant Bet v 1 allergen or a composition, wherein an IgE containing sample of a subject is mixed with said mutant or said composition and assessed for the level of reactivity between the IgE in said sample and said mutant.

### DETAILED DESCRIPTION OF THE INVENTION

In connection with the present invention the expression “reduce the specific IgE binding capability as compared to the IgE binding capability of the naturally occurring allergen” means that the reduction is measurable in a statistically significant manner (p<0.05) in at least one immunoassay using serum from a subject allergic to the natural-occurring allergen. Preferably, the IgE binding capability is reduced by at least 10%, more preferably at least 30%, more preferably at least 50%, and most preferably at least 70%.

The expression “surface-exposed amino acid” means that the amino acid residue is located at the surface of the three-dimensional structure in such a manner that when the allergen is in solution at least a part of at least one atom of the amino acid residue is accessible for contact with the surrounding solvent. Preferably, the amino acid residue in the three-dimensional structure has a solvent (water) accessibility of at least 20%, suitably at least 30%, more suitably at least 40% and most preferably at least 50%.

The expression “solvent accessibility” is defined as the area of the molecule accessible to a sphere with a radius comparable to a solvent (water, r=1.4 Å) molecule. The expressions “surface-exposed” and “solvent-exposed” are used interchangeably.

“Group of amino acids” should be understood as division of surface exposed amino acids suitable for mutation into groups. Each group represents a number of surface exposed amino acid residues that are found within a limited area on the surface of the allergen. An individual group comprises a number of amino acids that are part of at least one epitope. An individual group may also cover an area that comprises an entire epitope. One or more mutations within a single group is defined as one primary mutation. A mutated allergen with at least four primary mutations thus ensures that several epitopes will have a lowered IgE binding affinity. Mutation of amino acids from at least four groups may furthermore ensure an approximately even distribution of mutations on the molecular surface and ensure that several epitopes are mutated and thus resulting in a lowered IgE binding affinity of several epitopes compared to mutants with less than four mutations.

The expression “the taxonomic species from which said naturally occurring allergen originates” means species within the taxonomic genus, preferably the subfamily, more preferably the family, more preferably the superfamily, more preferably the legion, more preferably the suborder and most preferably the order from which said naturally occurring allergen originates.

The expression “moderately altered epitopes” means epitopes that retain essentially the same tertiary structure and surface topography as the corresponding unmutated epitopes. Moderate alterations are, generally speaking, achieved by exchanging an amino acid with another amino acid with similar chemical characteristics as the original amino acid. One way of achieving this is by exchanging one or more surface exposed amino acids with amino acids that might be found within the taxonomic order wherein the naturally occurring allergen is found. A moderately altered epitope might also contain amino acid substitutions where one or more of the substituted amino acid is not found within the taxonomic order wherein the naturally occurring allergen is found, as long as the substitution only slightly affects the tertiary structure of the epitope and/or the IgE binding affinity. The mutated allergen can be evaluated with respect to e.g. structure and IgE binding affinity subsequently. As opposed to the moderately altered epitopes are epitopes that are altered in a more drastic manner, e.g. mutations that significantly reduce the IgE binding affinity. Typically, drastic alterations of epitopes comprise amino acid substitutions where one or more amino acids have been exchanged with amio acids with different chemical properties.

Furthermore, the expression “the mutant allergen having essentially the same α-carbon backbone tertiary structure as the naturally occurring allergen” means that when comparing the structures of the mutant and the naturally occurring allergen, the average root mean square deviation of the atomic coordinates is preferably below 2 Å. Conservation of α-carbon backbone tertiary structure is best determined by obtaining identical structures by x-ray crystallography or NMR before and after mutagenesis. In absence of structural data describing the mutant indistinguishable CD-spectra or immunochemical data, e.g. antibody reactivity, may render conservation of α-carbon backbone tertiary structure probable, if compared to the data obtained by analysis of a structurally determined molecule.

In connection with the present invention the expression “mutation” means the deletion, substitution or addition of an amino acid in comparison to the amino acid sequence of the naturally occurring allergen. The terms “mutation” and “substitution” are used interchangeably. A recombinant mutated Bet v 1 allergen according to the invention may furthermore comprise amino acid insertions or amino acid deletion in particular surface exposed regions of the molecules e.g. “loop regions”. Loop regions connect secondary structure elements e.g. β-sheet, α-helixes and random coil structures. Loop regions in Bet v 1 are: Val12 to ala16, val33 to ser40, glu45 to Thr52, pro54 to tyr66, his76 to asn78, gly89 to glu96, val105 to gly111, thr122 to glu131. Mutant variants may comprise 1-5, more preferable 1-3 most preferably 1-2 substitutions in a loop region.

A primary mutation is defined as one or more mutations within a single group of surface exposed amino acids suitable for substitution. Each group of at least one mutated amino acids will have reduced IgE binding affinity as compared to the same group without mutations. Preferably, the recombinant allergen according to the invention comprises from 5 to 10, preferably from 6 to 10, more preferably from 7 to 10, and most preferably from 8 to 10 primary mutations.

Secondary mutations are defined as additional mutations within a single group. The recombinant allergen preferably comprises a number of secondary mutations, which each reduce the specific IgE binding capability of the mutated allergen as compared to the binding capability of the said naturally occurring allergen. Thus, a primary mutation that comprises several secondary mutations will in many cases have a more reduced IgE binding affinity than a primary mutation that has only one mutation. The recombinant allergen according to the invention comprises from 1 to 15, preferably 1-10 and most preferably 1-5 secondary mutations per primary mutation.

Conserved residues: Conserved residues in the naturally occurring allergen are conserved with more than 70%, preferably 80% and most preferably 90% identity in all known homologous proteins within the species from which said allergen originates. Amino acid residues that are highly solvent exposed and conserved constitute targets for substitution.

Another way of assessing the reduced IgE binding and the reduced ability of mediating cross-linking of the mutant are the capability of the mutant to initiate Histamine Release (HR). The release of Histamine can be measured in several Histamine releasing assays. The reduced Histamine release of the mutants originates from reduced affinity toward the specific IgE bound to the cell surface as well as their reduced ability to facilitate cross-linking. HR is preferably reduced by 5-100%, more preferably 25-100%, more preferably 50-100% and most preferably 75-100% for the mutants of the invention in comparison to the naturally occurring allergens.

In a preferred embodiment of the invention, a surface region comprising no mutation or only moderate mutations has an area of 800 Å², preferably 600 Å², more preferably 500 Å² and most preferably 400 Å². Typically, a surface region with an area of 800 Å² comprising no mutation or only moderate mutations comprises atoms of 15-25 amino acid residues.

In another embodiment, at least one of the amino acid residues to be incorporated into the mutant allergen does not occur in the same position in the amino acid sequence of any known homologous protein within the taxonomic genus, preferably the subfamily, more preferably the family, more preferably the superfamily, more preferably the legion, more preferably the suborder and most preferably the order from which said naturally occurring allergen originates.

According to the invention, the surface-exposed amino acid residues are ranked with respect to solvent accessibility, and at least four amino acids among the more solvent accessible ones are substituted.

In a further embodiment, a recombinant allergen is characterised in that the surface-exposed amino acid residues are ranked with respect to degree of conversation in all known homologous proteins within the species from which said naturally occurring allergen originates, and that one or more surface exposed amino acids among the more conserved ones are substituted.

The principle disclosed in the present invention comprises mutation of surface exposed amino acid residues selected from at least four groups of amino acids, wherein each group represents separate areas on the surface on the molecule. This principle may also be applied to allergens other than Bet v 1. A recombinant allergen according to the invention may suitably be a mutant of an inhalation allergen originating i.a. from trees, grasses, herbs, fungi, house dust mites, cockroaches and animal hair and dandruff. Important pollen allergens from trees, grasses and herbs are such originating from the taxonomic orders of Fagales, Oleales and Pinales including i.a. birch (Betula), alder (Alnus), hazel (Corylus), hornbeam (Carpinus) and olive (Olea), the order of Poales including i.a. grasses of the genera Lolium, Phleum, Poa, Cynodon, Dactylis and Secale, the orders of Asterales and Urticales including i.a. herbs of the genera Ambrosia and Artemisia. Important inhalation allergens from fungi are i.a. such originating from the genera Alternaria and Cladosporium. Other important inhalation allergens are those from house dust mites of the genus Dermatophagoides, those from cockroaches and those from mammals such as cat, dog and horse. Further, recombinant allergens according to the invention may be mutants of venom allergens including such originating from stinging or biting insects such as those from the taxonomic order of Hymenoptera including bees (superfamily Apidae), wasps (superfamily Vespidea), and ants (superfamily Formicoidae).

Specific allergen components include e.g. Bet v 1 (B. verrucosa, birch), Aln g 1 (Alnus glutinosa, alder), Cor a 1 (Corylus avelana, hazel) and Car b 1 (Carpinus betulus, hornbeam) of the Fagales order. Others are Cry j 1 (Pinales), Amb a 1 and 2, Art v 1 (Asterales), Par j 1 (Urticales), Ole e 1 (Oleales), Ave e 1, Cyn d 1, Dac g 1, Fes p 1, Holl 1, Lol p 1 and 5, Pas n 1, Phl p 1 and 5, Poa p 1, 2 and 5, Sec c 1 and 5, and Sor h 1 (various grass pollens), Alt a 1 and Cla h 1 (fungi), Der f 1 and 2, Derp 1 and 2 (house dust mites, D. farinae and D. pteronyssinus, respectively), Eur m 1 (mite, Euroglyphus maynel), (Lep d 1 and 2 (Lepidoglyphus destructor; storage mite), Bla g 1 and 2, Per a 1 (cockroaches, Blatella germanica and Periplaneta americana, respectively), Fel d 1 (cat), Can f 1 (dog), Equ c 1, 2 and 3 (horse), Apis m 1 and 2 (honeybee), Ves v 1, 2 and 5, Pol a 1, 2 and 5 (all wasps) and Sol i 1, 2, 3 and 4 (fire ant).

Some Examples of Adding Further Substitutions to a Given Mutant

In one embodiment of the invention further substitutions are added to mutant allergens in such a way that it is ensured that the substitutions of the final mutant allergen are essentially evenly distributed on the molecular surface and that the groups contain essentially the same number of introduced mutations. This is illustrated in the following examples where mutants comprising specific substitutions preferably should have added further substitutions from a list where the succession of amino acids reflects the preferred order of adding more substitutions. Without limiting the present invention, these examples represent one application of how to design mutants and the man skilled in the art might very well choose a somewhat different approach in order to ensure an even distribution of substitutions. Mutants may thus be designed comprising one or more substitutions from the lists given below.

Bet v 1 mutant (“3004A”) allergens comprising the following substitutions: Y5V, E45S, N78K, K97S, K103V, K134E, +160N. Further substitutions may comprise one or more of the following: E8 or K115, D125 or H126, E138 or K137 or E141, D25 or N28, E87 or K55, S155 or H154 or N159, N47 or P50 or H76 or N43 or I44 or R70, E73 or P50 or D72, A130, N28 or D25, P108, V2 or K119 or N4 or E6 or E96.

Bet v 1 mutant (“3004B”) allergens comprising the following substitutions: Y5V, E45S, L62F, N78K, K97S, K103V, K134E, +160N. Further substitutions may comprise one or more of the following: T10P, K65N, N28 or D25 or K32Q or E141X or K137X or E138X, D125X or K1231 or H126, P108X or D109N, E42S or K55X or I44X or N43X, E73X or D72X, E87X, E96X or K119, A130X, V2X or E6X, E8X or K115, N47X or P50X or R70X or H76X or T77A, S155X or D156H or N159X, E6X or V2X.

Bet v 1 allergen mutants (“3005A”) comprising the following substitutions: Y5V, N28T, K32Q, E45S, L62F, N78K, K97S, K103V, K134E, +160N. Further substitutions may comprise one or more of the following: E8X or K115X, D125 or H126, E138X or K137X or E141X, E87X or K55X, S155Xor H154X or N159X, N47X or P50X or H76X or N43X or I44X or R70X, E73X or P50X or D72X, A130X, D25X, P108X, V2X or K119X or N4X or E6X or E96X.

Bet v 1 allergen mutants (“3005B”) comprising the following substitutions: Y5V, N28T, K32Q, E45S, L62F, N78K, K97S, K103V, K134E, +160N. Further substitutions may comprise one or more of the following: T10P, K65N, E141X or K137X or E138X, D125X or K123I or H126X, P108X or D109N, E42S or K55X or I44X or N43X, E73X or D72X, E87X, E96X or K119X, A130X, V2X or E6X, E8X or K115X, N47X or P50X or R70X or H76X or T77A, S155X or D156H or N159X, E6X or V2X.

Bet v 1 allergen mutants (“3006A”) comprising the following substitutions: Y5V, N28T, K32Q, E45S, N78K, E87S, K97S, K103V, K134E, N159G, +160N. Further substitutions may comprise one or more of the following: K55, A138 or K137 or E141, D125 or H126, P108, V2 or N4 or K119 or E6, S155 or H154, N47 or P50 or H76, E73, R70, A130, E8 or K115, E96.

Bet v 1 allergen mutants (“3006B”) comprising the following substitutions: Y5V, N28T, K32Q, E45S, N78K, E87S, K97S, K103V, K134E, N159G, +160N. Further substitutions may comprise one or more of the following: K65N, T10P, D125, K123I, P108, D109N, N47 or P50 or H76, E138 or K137 or E141, E42S or K55 or I44 or N43, S155 or D156H, E73 or D72, E6 or V2, E96.

Bet v 1 allergen mutants (“3007A”) comprising the following substitutions: Y5V, N28T, K32Q, E45S, L62F, N78K, K97S, K103V, P108G, D125Y, K134E, +160N. Further substitutions may comprise one or more of the following: E87, E141, E138, K55, N47 or N43X or 144 or H76, S155 or H154, A130, E8, E73, V2 or K119, D25.

Bet v 1 allergen mutants (“3007B”) comprising the following substitutions: Y5V, N28T, K32Q, E45S, L62F, N78K, K97S, K103V, P108G, D125Y, K134E, +160N. Further substitutions may comprise one or more of the following: K65N, T10P or E8, E87, S155 or D156H, E138, E141, E42S, A130, E8 or T10P, N47, H76, R70, E96.

Bet v 1 allergen mutants (“3008A”) comprising the following substitutions: Y5V, N28T, K32Q, E45S, L62F, E73S, E96S, P108G, D125Y, N159G, +160N. Further substitutions may comprise one or more of the following: E134, N78, E87, K119, E8, K55, E138, E141, S155, N47, E6, K103, D25, A130, V2, R70.

Bet v 1 allergen mutants (“3008B”) comprising the following substitutions: Y5V, N28T, K32Q, E45S, L62F, E73S, E96S, P108G, D125Y, N159G, +160N. Further substitutions may comprise one or more of the following: K65N or K55, T10P or E8 or E141, E138 or K134, E87, E42S or K55 or I44, S155 or D156H, N78, K119 or V2 or N4, N47 or P50, H76 or T77A, A130, D25, E6 or K115 or K103.

Bet v 1 allergen mutants (“3009A”) comprising the following substitutions: Y5V, N28T, K32Q, E45S, L62F, E96S, P108G, +160N. Further substitutions may comprise one or more of the following: E134, N78, E87, K1 19, E8, K55, E138, E141, S155, N47, E6, K103, D25, A130, V2, R70.

Bet v 1 allergen mutants (“3009B”) comprising the following substitutions: Y5V, N28T, K32Q, E45S, L62F, E96S, P108G, +160N. Further substitutions may comprise one or more of the following: N78 or T77A, K103, E134 or E138, K65N or K55, T10P, D125 or H126, S155 or D156H or HIS154, K119 or V2, E87, N47 or P50 or H76, E42S or K55, I44 or N43, A130.

Loop Mutations:

In another embodiment of the invention mutant allergens according to the invention furthermore comprise amino acid insertions or amino acid deletion in particular surface exposed regions of the molecules e.g. loop regions. Loop regions connect secondary structure elements e.g. β-sheet, α-helixes and random coil structures. Loop regions in Bet v 1 are: val12 to ala16, val33 to ser40, glu45 to Thr52, pro54 to tyr66, his76 to asn78, gly89 to glu96, val105 to gly111, thr122 to glu131. Mutant variants according to this embodiment comprise 1-5, more preferable 1-3 most preferably 1-2 substitutions in a loop region. In a preferred embodiment, mutant allergens comprise at least four mutations selected from the 10 groups as well as a number of additional “loop-mutations”. Examples of such “loop mutations” , wherein x represents an added amino acid residue, are:

Bet v 1 (3007-L1) with an amino acid insertion between residue E60 and residue G61 (SEQ ID NO 1):

GVFNVETETTSVIPAARLFKAFILDGDTLFPQVAPQAISSVENISGNGGP
GTIKKISFPExGFPFKYVKDRVDEVDHTKFKYNYSVIEGGPIGDTLESIS
NEIVIVATGDGGSILKISNKYHTKGYHEVKAEQVEASKEMGETLLRAVES
YLLAHSDAYNN

Bet v 1 (3007-L2) with amino acid insertion between residue D93 and residue T94 (SEQ ID NO 2):

GVFNVETETTSVIPAARLFKAFILDGDTLFPQVAPQAISSVENISGNGGP
GTIKKISFPEGFPFKYVKDRVDEVDHTKFKYNYSVIEGGPIGDxTLESIS
NEIVIVATGDGGSILKISNKYHTKGYHEVKAEQVEASKEMGETLLRAVES
YLLAHSDAYNN

Bet v 1 (3007-L3) with amino acid insertion between residue V12 and residue I13 (SEQ ID NO 3):

GVFNVETETTSVxIPAARLFKAFILDGDTLFPQVAPQAISSVENISGNGG
PGTIKKISFPEGFPFKYVKDRVDEVDHTKFKYNYSVIEGGPIGDTLESIS
NEIVIVATGDGGSILKISNKYHTKGYHEVKAEQVEASKEMGETLLRAVES
YLLAHSDAYNN

Bet v 1 (3007-L4) with amino acid insertions between residue I56 and residue S57 and between residue K65 and residue T66 (SEQ ID NO 4):

GVFNVETETTSVIPAARLFKAFILDGDTLFPQVAPQAISSVENISGNGGP
GTIKKIxSFPExGFPFKYVKDRVDEVDHTkFKYNYSVIEGGPIGDTLESI
SNEIVIVATGDGGSILKISNKYHTKGYHEVKAEQVEASKEMGETLLRAVE
SYLLAHSDAYNN

Bet v 1 (3007-L5) with amino acid deletion of residue G111 (SEQ ID NO 5):

GVFNVETETTSVIPAARLFKAFILDGDTLFPQVAPQAISSVENISGNGGP
GTIKKISFPEGFPFKYVKDRVDEVDHTKFKYNYSVIEGGPIGDTLESISN
EIVIVATGDGSILKISNKYHTKGYHEVKAEQVEASKEMGETLLRAVESYL
LAHSDAYNN

Method of Preparing a Recombinant Allergen According to the Invention

The surface-exposed amino acids suitable for substitution in accordance with the present invention may be identified on the basis of information of their solvent (water) accessibility, which expresses the extent of surface exposure. A preferred embodiment of the method of the invention is characterised in ranking the said identified amino acid residues with respect to solvent accessibility and substituting one or more amino acids among the more solvent accessible ones.

Furthermore, another embodiment of the method of the invention is characterised in ranking the identified amino acid residues with respect to degree of conversation in all known homologous proteins within the species from which said naturally occurring allergen originates and substituting one or more amino acids among the more conserved ones.

A further preferred embodiment of the method of the invention comprises selecting the identified amino acids so as to form a mutant allergen, which has essentially the same α-carbon backbone tertiary structure as said naturally occurring allergen.

Another preferred embodiment of the method of the invention is characterised in that the substitution of amino acid residues is carried out by site-directed mutagenesis.

An alternative preferred embodiment of the method of the invention is characterised in that the substitution of amino acid residues is carried out by DNA shuffling or by setting up a library comprising suitable positions and their preferred substitutents.

Criteria for Substitution

For molecules for which the tertiary structure has been determined (e.g. by x-ray crystallography, or NMR electron microscopy), the mutant carrying the substituted amino acid(s) should preferably fulfil the following criteria:

1. The overall α-carbon backbone tertiary structure of the recombinant mutant is preferably conserved. Conserved is defined as an average root mean square deviation of the atomic coordinates below 2 Å when comparing the structures of the mutated allergen and the naturally occurring allergen. This is important for two reasons: a) It is anticipated that the entire surface of the natural allergen constitutes an overlapping continuum of potential antibody-binding epitopes. The majority of the surface of the molecule is not affected by the substitution(s), and thus retain its antibody-binding inducing properties, which is important for the generation of new protective antibody specificities being directed at epitopes present also on the natural allergen. b) Stability, both concerning shelf-life and upon injection into body fluids.

Conservation of α-carbon backbone tertiary structure is best determined by obtaining identical structures by x-ray crystallography or NMR before and after mutagenesis. In absence of structural data describing the mutant indistinguishable CD-spectra or immunochemical data, e.g. antibody reactivity, may render conservation of α-carbon backbone tertiary structure probable, if compared to the data obtained by analysis of a structurally determined molecule.

2. The amino acids to be substituted are preferably located at the surface, and thus accessible for antibody-binding. Amino acids located on the surface in the three-dimensional structure usually have a solvent (water) accessibility of at least 20%, suitably 20-80%, more suitably 30-80%. Solvent accessibility is defined as the area of the molecule accessible to a sphere with a radius comparable to a solvent (water, r=1.4 Å) molecule.

3. The substituted amino acids are selected from at least four groups. Each group represents a number of preferred surface exposed amino acid residues that are found within a limited area on the surface of the allergen.

One or more mutations within a single group is defined as one primary mutation. An individual group comprises a number of amino acids that are part of at least one epitope. An individual group may also comprise an entire epitope. A mutated allergen with at least four primary mutations thus ensures that several epitopes will have a lowered IgE binding affinity. Mutation of amino acids from at least four groups furthermore ensures an approximately even distribution of mutations on the molecular surface and it ensures that several epitopes will become mutated and thus obtaining a lowered IgE binding affinity of several epitopes.

With an object of essentially retaining the three-dimensional structure of the allergen, the amino acid to be incorporated may be selected on the basis of a comparison with a protein, which is a structural homologue to the allergen, e.g. a protein, which belongs to the same taxonomic order as the allergen, and which does not have any cross-reactivity with the allergen.

Vaccines:

Preparation of vaccines is generally well known in the art. Vaccines are typically prepared as injectables either as liquid solutions or suspensions. Such vaccine may also be emulsified or formulated so as to enable nasal administration as well as oral, including buccal and sublingual, administration. The immunogenic component in question (the recombinant allergen as defined herein) may suitably be mixed with excipients which are pharmaceutically acceptable and further compatible with the active ingredient. Examples of suitable excipients are water, saline, dextrose, glycerol, ethanol and the like as well as combinations thereof. The vaccine may additionally contain other substances such as wetting agents, emulsifying agents, buffering agents or adjuvants enhancing the effectiveness of the vaccine.

Vaccines are most frequently administered parenterally by subcutaneous or intramuscular injection. Formulations which are suitable for administration by another route include oral formulations and suppositories. Vaccines for oral administration may suitably be formulated with excipients normally employed for such formulations, e.g. pharmaceutical grades of mannitol, lactose, starch, magnesium stearate, sodium saccharine, cellulose, magnesium carbonate and the like. The composition can be formulated as solutions, suspensions, emulsions, tablets, pills, capsules, sustained release formulations, aerosols, powders, or granulates.

The vaccines are administered in a way so as to be compatible with the dosage formulation and in such amount as will be therapeutically effective and immunogenic. The quantity of active component contained within the vaccine depends on the subject to be treated, i.a. the capability of the subject's immune system to respond to the treatment, the route of administration and the age and weight of the subject. Suitable dosage ranges can vary within the range from about 0.0001 μg to 1000 μg.

As mentioned above, an increased effect may be obtained by adding adjuvants to the formulation. Examples of such adjuvants are aluminum hydroxide and phosphate (alum) or calcium phosphate as a 0.05 to 0.1 percent solution in phosphate buffered saline, synthetic polymers of sugars or polylactid glycolid (PLG) used as 0.25 percent solution. Mixture with bacterial cells such as C. parvum, endotoxins or lipopolysaccharide components of gram-negative bacteria, emulsion in physiologically acceptable oil vehicles such as mannide monoaleate (Aracel A) or emulsion with 20 percent solution of a perfluorocarbon (e.g. Fluosol-DA) used as a block substitute may also be employed. Oil emulsions, such as MF-59 may also be used. Other adjuvants such as Freund's complete and incomplete adjuvants as well as saponins, such as QuilA, Qs-21 and ISCOM, and RIBI may also be used.

Most often, multiple administrations of the vaccine will be necessary to ensure an effect. Frequently, the vaccine is administered as an initial administration followed by subsequent inoculations or other administrations. The number of vaccinations will typically be in the range of from 1 to 50, usually not exceeding 35 vaccinations. Vaccination will normally be performed from biweekly to bimonthly for a period of 3 months to 5 years. This is contemplated to give desired level of prophylactic or therapeutic effect.

The recombinant allergen may be used as a pharmaceutical preparation, which is suitable for providing a certain protection against allergic responses during the period of the year where symptoms occur (prophylaxis). Usually, the treatment will have to be repeated every year to maintain the protective effect. Preparations formulated for nasal, oral and sublingual application are particular suited for this purpose.

DNA According to the Invention

The DNA sequence of the invention is a mutant of a DNA sequence encoding a naturally occurring Bet v 1 allergen. Examples of naturally occurring Bet v 1 molecules are SEQ ID NO 1 (data base accession number Z80104) and SEQ ID NO 2 (data base accession number P15494). Other Bet v 1 variants include Bet v 1 sequences with the following data base accession numbers: P15494=X15877=Z80106, Z80101, AJ002107, Z72429, AJ002108, Z80105, Z80100, Z80103, AJ001555, Z80102, AJ002110, Z72436, P43183=X77271, Z72430, AJ002106, P43178=X77267, P43179=X77268, P43177=X77266, Z72438, P43180=X77269, AJ001551, P43185=X77273, AJ001557, Z72434, AJ001556, Z72433=P43186, AJ001554, X81972, Z72431, P45431=X77200, P43184=X77272, P43176=X77265, S47250, S47251, Z72435, Z72439, Z72437, and S47249.

Preferably, the DNA derivative is obtained by site-directed or random or semi random mutagenesis of the DNA encoding the naturally occurring allergen.

A “mutant library” is a library of mutant allergens. This library is constructed using degenerated DNA oligonucleotide primers that allow introduction of none, a single or several different amino acid residues in each position. Such a library approach allows amino acid residues to be either conservatively or non-conservatively substituted. As structural integrity may be less affected by conserved mutations introduction of such “soft” or moderate mutations in certain positions may increase the changes of generating stable mutants. Construction of mutant libraries may be one way to overcome problems with protein stability of mutated allergens caused by a single or a certain combination of mutations. A “semi-random library” means that positions to be mutated are confined to amino acid residues, which are surface exposed.

This approach further enhances the probability of obtaining stable mutant allergens. “Semi-random” can also mean that the primers designed allow for a selected number of amino acid residues to be substituted in the chosen position. The two semi-random approaches can be used independently or in combination. Theoretically, a library according to the invention comprises a number of rBet v 1 mutant allergens each having at least 4 amino acid substitutions compared to non-mutated Bet v 1.

In one embodiment a semi-random library based on rBet v 1 (2744) (mutated in positions Y5, E42, E45, N78, K103, K123, K134, D156, +160) and rBet v1 (2628) (mutated in positions Y5, E45, K65, K97, K134) was constructed where an additional 7 target positions on the allergen surface were targeted: T10, K20, Q36, E73, E87, K129 and S149. These seven positions were selected from surface areas that are outside coherent surface areas that are common among Fagales allergens. The library was based on the use of degenerated DNA oligonucleotide primers allowing introduction of several different amino acid residues in each position. In addition, several mutated amino acid residue positions in rBet v 1 (2744) and rBet v1 (2628) could either be maintained or mutated back to the residues found in WT rBet v 1.2801.

I another embodiment a semi-random library based on rBet v 1 (2744) and rBet v1 (2628) and rBet v 1 (2595) i.e. N28, K32, E45, P108 was constructed where an additional 7 target positions on the allergen surface were targeted: T10, K20, Q36, E73, E87, K129 and S149.

Mutants:

Examples of specific Bet v 1 allergen mutants according to the present invention are listed below. Mutated amino acid positions are indicated in bold small print:

| | |
|---|---|
| Bet v 1 (“3004”): | |
| GVFNvETETTSVIPAARLFKAFILDGDNLFPKVAPQ | (SEQ ID NO 6) |
| AISSVsNIEGNGGPGTIKKISFPEGfPFKYVKDRVD | |
| EVDHTkFKYNYSVIEGGPIGDTLEsISNEIvIVATP | |
| DGGSILKISNKYHTKGDHEVKAEQVeASKEMGETLL | |
| RAVESYLLAHSDAYNn | |
| Bet v 1 (“3005”): | |
| GVFNvETEITSVIPAARLFKAFILDGDtLFPqVAPQ | (SEQ ID NO 7) |
| AISSVENIsGNGGPGTIKKISFPEGfPFKYVKDRVD | |
| EVDHTkFKYNYSVIEGGPIGDTLEsISNEIvIVATP | |
| DGGSILKISNKYHTKGDHEVKAEQVeASKEMGETLL | |
| RAVESYLLAHSDAYNn | |
| Bet v 1 (“3007”): | |
| GVFNvETETTSVIPAARLFKAFILDGDtLFPqVAPQ | (SEQ ID NO 8) |
| AISSVENIsGNGGPGTIKKISFPEGfPFKYVKDRVD | |
| EVDHTkFKYNYSVIEGGPIGDTLEsISNEIvIVATg | |
| DGGSILKISNKYHTKGyHEVKAEQVeASKEMGETLL | |
| RAVESYLLAHSDAYNn | |
| Bet v 1 (“3009”): | |
| GVFNvETETTSVIPAARLFKAFILDGDtLFPqVAPQ | (SEQ ID NO 9) |
| AISSVENIsGNGGPGTIKKISFPEGfPFKYVKDRVD | |
| EVDHTkFKYNYSVIEGGPIGDTLsKISNEIKIVATg | |
| DGGSILKISNKYHTKGDHEVKAEQVKASKEMGETLL | |
| RAVESYLLAHSDAYNn | |
| Bet v 1 (“3006”): | |
| GVFNvETETTSVIPAARLFKAFILDGDtLFPqVAPQ | (SEQ ID NO 10) |
| AISSVENIsGNGGPGTIKKISFPEGfPFKYVKDRVD | |
| EVDHTkFKYNYSVIEGGPIGDTLEsISNEIvIVATP | |
| DGGSILKISNKYHTKGDHEVKAEQVeASKEMGETLL | |
| RAVESYLLAHSDAYgn | |
| Bet v 1 (“3008”): | |
| GVFNvETETTSVIPAARLFKAFILDGDtLFPkVAPQ | (SEQ ID NO 11) |
| AISSVENIsGNGGPGTIKKISFPEGfPFKYVKDRVD | |
| sVDHTNFKYNYSVIEGGPIGDTLsKISNEIKIVATg | |
| DGGSILKISNKYHTKGyHEVKAEQVKASKEMGETLL | |
| RAVESYLLAHSDAYgn | |

The present invention furthermore comprises the following specific mutants:

| | |
|---|---|
| Bet v 1 (“3005-7”): | |
| Y5V, N28T, K32Q, E45S, N78K, K97S, K103V, K134E, +160N, E8S, D125Y, | |
| E141S, D25T, E87A, S155T, N47K, K55N. | |
| GVFNvETsTTSVIPAARLFKAFILtGDtLFPqVAPQAISSVENIsGkGGPGTIKnIS | (SEQ ID NO 12) |
| FPEGLPFKYVKDRVDEVDHTkFKYNYSVIaGGPIGDTLEsISNEIVlVATPDGG | |
| SILKISNKYHTKGyHEVKAEQVeASKEMGsTLLRAVESYLLAHtDAYNn | |
| Bet v 1 (“3005-12”): | |
| Y5V, N28T, K32Q, E45S, N78K, K97S, K103V, K134E, +160N, E8S, D125Y, | |
| E14IS, D25T, E87A, S155T, N47K, K55N, E73T, A13OV, P108G, V2L. | |
| GIFNvETsTTSVIPAARLFKAFILtGDtLFPqVAPQAISSVENIsGkGGPGTIKnIS | (SEQ ID NO 13) |
| FPEGLPFKYVKDRVDtVDHTkFkYNYSVIaGGPIGDTLEsISNEIvIVATgDGGS | |
| ILKISNKYHTKGyHEVKvEQVeASKEMGsTLLRAVESYLLAHtDAYNn | |
| Bet v 1 (“3005-22”): | |
| Y5V, N28T, K320, E45S, N78K, K97S, K103V, K134E, +160N, T10K, K65N, | |
| E141N, K1231, D1O9N, E42S, EJ3T, E87A, V2L, N47K. | |
| GIFNvETETpSVIPAARLFKAFILDGDtLFPqVAPQAISSVsNIsGkGGPGTIKKI | (SEQ ID NO 14) |
| SFPEGLPFnYVKDRVDtVDHTtFKYNYSVIaGGPIGDTLEsISNEIvIVATPnGG | |
| SILKISNKYHTiGDHEVKAEQVeASKEMGnTLLRAVESYLLAHSDAYNn | |
| Bet v 1 (“3005-27”): | |
| Y5V, N28T, K320, E45S, N78K, K97S, K103V, K134E, +160N, T10K, K65N, | |
| E141N, K1231, D1O9N, E42S, E73T, E87A, K119N, A13OV, V2L, E8S, N47K, | |
| D156H, E6S. | (SEQ ID NO 15) |
| GIFNvsTsTpSVIPAARLFKAFILDGDtLFPqVAPQAISSVsNIsGkGGPGTIKKIS | |
| FPEGLPFnYVKDRVDtVDHTtFKYNYSVIaGGPIGDTLEsISNEIvIVATPnGGSI | |
| LKISNKYHTiGDHEVKAEQVeASKEMGnTLLRAVESYLLAHShAYNn | |
| Bet v 1 (“3007-6”): | |
| Y5V, N28T, K32S, E45S, N78K, K97S K103V, P108G, D125Y, K134E, | |
| +160N, E87A, E141N, K55N, N47K, S155T. | |
| GVFNvETETTSVIPAARLFKAFILDGDtLFPqVAPQAISSVENIsGkGGPGTIKnI | (SEQ ID NO 16) |
| SFPEGLPFKYVKDRVDEVDHTkFKYNYSVIaGGPIGDTLEsISNEIvIVATgDG | |
| GSILKISNKYHTKGyHEVKAEQVeASKEMGnTLLRAVESYLLAHtDAYNn | |
| Bet v 1 (“3007-10”): | |
| Y5V, N2BT, K325, E455, N78K, K97S K103V, P108G, D125Y, K134E, | |
| +160N, E87A, E141N, K55N, N47K, 5155T, AI3OV, EBS, E73T, V2L. | |
| GIFNvETsTTSVIPAARLFKAFILDGDtLFPqVAPQAISSVENIsGkGGPGTIKnIS | (SEQ ID NO 17) |
| FPEGLPFKYVKDRVDtVDHTkFKYNYSVIaGGPIGDTLEsISNEIvIVATgDGGS | |
| ILKISNKYHTKGyHEVKvEQVeASKEMGnTLLRAVESYLLAHtDAYNn | |
| Bet v 1 (“3007-17”): | |
| Y5V, N28T, K32Q, E45S, N78K, K97S, K103V, P108G, D125Y, K134E, | |
| +160N, K65N, T10P E87A, D156H, EI41N, E42S. | |
| GVFNvETETpSVIPAARLFKAFILDGDtLFPqVAPQAISSVsNIsGNGGPGTIKK | (SEQ ID NO 18) |
| ISFPEGLPFnYVkDRVDEVDHIkFKYNYSVIaGGPIGDTLEsISNEIvIVATgDG | |
| GSILKISNKYHTKGyHEVKAEQVeASKEMGnTLLRAVESYLLAHShAYNn | |
| Bet v 1 (“3007-22”): | |
| Y5V, N28T, K32Q, E45S, N78K, K97S, K103V, P108G, D125Y, K134E, | |
| +160N, K65N, T10P E87A, D156H, E141N, E42S, A130V, E8S, N47K, | |
| H76T, V2L. | |
| GIFNvETsTpSVIPAARLFKAFILDGDtLFPqVAPQAISSVsNIsGkGGPGTIKKIS | (SEQ ID NO 19) |
| FPEGLPFnYVKDRVDEVDtTkFKYNYSVIaGGPIGDTLEsISNEIvIVATgDGGS | |
| ILKISNKYHTKGyHEVKvEQVeASKEMGnTLLRAVESYLLAHShAYNn | |
| Bet v 1 (“3008-8”): | |
| Y5V, N28T, K32Q, E455, E73S, E965, P108G, D125Y, N159G, +160N, | |
| K134E, N78K, E87A, KI19N, E85, K55N, E141N, N47K. | |
| GVFNvETsTTSVIPAARLFKAFILDGDtLFPqVAPQAISSVENIsGkGGPGTIKnI | (SEQ ID NO 20) |
| SFPEGLPFKYVKDRVDsVDHTkFKYNYSVIaGGPIGDTLsKISNEIKIVATgDG | |
| GSILKISNnYHTKGyHEVKAEQVeASKEMGnTLLRAVESYLLAHSDAYgn | |
| Bet v 1 (“3008-13”): | |
| Y5V, N28T, K32Q, E455, E735, E965, P108G, D125Y, N159G, +160N, | |
| K134E, N78K, E87A, K119N, E85, K55N, E141N, N47K, 5156T, E65, | |
| K103V, A130V, V2L. | |
| GIFNvsTsTTSVIPAARLFKAFILDGDtLFPqVAPQAISSVENIsGkGGPGTIKnIS | (SEQ ID NO 21) |
| FPEGLPFKYVKDRVDsVDHTkFKYNYSVIaGGPIGDTLsKISNEIvIVATgDGG | |
| SILKISNnYHTKGyHEVKvEQVeASKEMGnTLLRAVESYLLAHtDAYgn | |
| Bet v 1 (“3008-20”): | |
| Y5V, N28T, K32Q, E455, E735, E96S, P108G, D125Y, N159G, +160N, | |
| K65N, T10P, E138N, E87A, E425, D156H, N78K. | |
| GVFNvETETpSVIPAARLFKAFILDGDtLFPqVAPQAISSVsNIsGNGGPGTIKK | (SEQ ID NO 22) |
| ISFPEGLPFnYVKDRVDsVDHTkFKYNYSVIaGGPIGDTLsKISNEIKIVATgDG | |
| GSILKISNKYHTKGyHEVKAEQVKASKnMGETLLRAVESYLLAHShAYgn | |
| Bet v 1 “3008-25”): | |
| Y5V, N28T, K32Q, E45S, E73S, E96S, P108G, D125Y, N159G, +160N, | |
| K65N, T10P, E138N, E87A, E42S, D156H, N78K, KI19N, N47K, T77A, | |
| E130V, K115N. | |
| GIFNvsTETpSVIPAARLFKAFILDGDtLFPqVAPQAISSVsNIsGNGGPGTIKKI | (SEQ ID NO 23) |
| SFPEGLPFnYVKDRVDsVDHTkFKYNYSVIaGGPIGDTLsKISNEIvIVATgDG | |
| GSILKISNKYHTKGyHEVKvEQVKASKnMGETLLRAVESYLLAHthAYgn | |
| Bet v 1 (“3009-9”): | |
| Y5V, N28T, K32Q, E45S, E96S, P108G, +160N, K134E, N78K, E87A, | |
| K119N, E8S, K55N, E138N, E141N, S155T, N47K, E6S, K103V, A130V, | |
| V2L, R70N, D125Y. | |
| GVFNvETsTTSVIPAARLFKAFILDGDtLFPqVAPQAISSyENIsGRGGPGTIKnI | (SEQ ID NO 24) |
| SFPEGLPFKYVKDRVDEVDHTkFKYNYSVIaGGPIGDTLsKISNEIKIVATgDG | |
| GSILKISNnYHTKGDHEVKAEQVeASKnMGnTLLRAVESYLLAHtDAYNn | |
| Bet v 1 (“3009-15”): | |
| Y5V, N28T, K320, E45S, E96S, P108G, +160N, K134E, N78K, E87A, | |
| K119N, E8S, K55N, E138N, E141N, S155T, N47K, E6S, K103V, A130V, | |
| V2L, R70N, D125Y. | |
| GIFNvsTsTTSVIPAARLFKAFILDGDtLFPqVAPQAISSVENIsGkGGPGTIKnIS | (SEQ ID NO 25) |
| FPEGLPFKYVKDnVDEVDHTkFKYNYSVIaGGPIGDTLsKISNEIvIVATgDGG | |
| SILKISNnYHTKGyHEVKvEQVeASKnMGnTLLRAVESYLLAHtDAYNn | |
| Bet v 1 (“3009-22”): | |
| Y5V, N28T, K32Q, E45S, E96S, P108G, +160N, T77A, K103V, E138N, | |
| K65N, T10P, D125Y, E42S. | |
| GVFNvETETpSVIPAARLFKAFILDGDtLFPqVAPQAISSVsNIsGNGGPGTIKK | (SEQ ID NO 26) |
| ISFPEGLPFnYVKDRVDEVDHaNFKYNYSVIEGGPIGDTLsKISNEIvIVATgD | |
| GGSILKISNKYHTKGyHEVKAEQVKASKnMGETLLRAVESYLLAHSDAYNn | |
| Bet v 1 (“3009-28”): | |
| Y5V, N28T, K32Q, E45S, E96S, P108G, +160N, T77A, K103V, E138N, | |
| K65N, T10P, D125Y, D156H, KI19N E87A, E42S, A130V. | |
| GVFNvETETpSVIPAARLFKAFILDGDtLFPqVAPQAISSVsNISGNGGPGTIKK | (SEQ ID NO 27) |
| ISFPEGLPFnYVKDRVDEVDHaNFKYNYSVIaGGPIGDTLsKISNEIvIVATgDG | |
| GSILKISNnYHTKGyHEVKvEQVKASKnMGETLLRAVESYLLAHShAYNn | |
| Bet v 1 clone (“3031”): | |
| GVFNVETETASVIPAARLFNAFILDGDTLFPQVAPQAISSVSNISGNGGPGTI | (SEQ ID NO 28) |
| KKISFPEGLPFNYVKDRVDSVDHTNFKYNYSVIEGGPIGDTLESISNEIVIVAT | |
| PDGGSILKISNKYHTIGDHEVEAEQVEASKEMGETLLRAVEPYLLAHSHAYN | |
| N | |
| Bet v 1 clone (“3032”): | |
| GVFNVETETASVIPAARLFLAFILDGDTLFPQVAPPAISSVSNISGNGGPGTI | (SEQ ID NO 29) |
| KKISFPEGLPFNYVKDRVDPVDHTKFKYNYSVIGGGPIGDTLESISNEIVIVAT | |
| PDGGSILKISNKYHTIGDHEVEAEQVEASKEMGETLLRAVEGYLLAHSHAYN | |
| N | |
| Bet v 1 clone (“3033”): | |
| GVFNVETETPSVIPAARLFHAFILDGDTLFPQVAPKAISSVSNISGNGGPGTI | (SEQ ID NO 30) |
| KKISFPEGLPFNYVKDRVDRVDHTKFKYNYSVIEGGPIGDTLESISNEIVIVAT | |
| PDGGSILKISNKYHTIGDHEVEAEQVEASKEMGETLLRAVEGYLLAHSHAYN | |
| N | |
| Bet v 1 clone (“3034”): | |
| GVFNVETETTSVIPAARLFHAFILDGDNLFPKVAPPAISSVSNISGNGGPGTI | (SEQ ID NO 31) |
| KKISFPEGLPFNYVKDRVDSVDHTKFKYNYSVIGGGPIGDTLESISNEIVIVAT | |
| PDGGSILKISNKYHTIGDHEVEAEQVEASKEMGETLLRAVERYLLAHSHAYN | |
| N | |
| Bet v 1 clone (“3035”): | |
| GVFNVETETPSVIPAARLFMAFILDGDTLFPQVAPPAISSVSNISGNGGPGTI | (SEQ ID NO 32) |
| KKISFPEGLPFNYVKDRVDSVDHTNFKYNYSVIGGGPIGDTLESISNEIVIVAT | |
| PDGGSILKISNKYHTIGDHEVEAEQVEASKEMGETLLRAVEAYLLAHSHAYN | |
| N | |
| Bet v 1 clone (“3036”): | |
| GVFNVETETPSVIPAARLFLAFILDGDNLFPKVAPPAISSVSNISGNGGPGTI | (SEQ ID NO 33) |
| KKISFPEGLPFNYVKDRVDTVDHTKFKYNYSVIGGGPIGDTLESISNEIVIVAT | |
| PDGGSILKISNKYHTIGDHEVEAEQVEASKEMGETLLRAVERYLLAHSHAYN | |
| N | |
| Bet v 1 clone (“3037”): | |
| GVFNVETETPSVIPAARLFQAFILDGDNLFPKVAPPAISSVSNISGNGGPGTI | (SEQ ID NO 34) |
| KKISFPEGLPFNYVKDRVDSVDHTNFKYNYSVIGGGPIGDTLESISNEIVIVAT | |
| PDGGSILKISNKYHTIGDHEVEAEQVEASKEMGETLLRAVEPYLLAHSHAYN | |
| N | |
| Bet v I clone (“3038”): | |
| GVFNVETETASVIPAARLFLAFILDGDNLFPKVAPPAISSVSNISGNGGPGTI | (SEQ ID NO 35) |
| KKISFPEGLPFNYVKDRVDGVDHTKFKYNYSVIDGGPIGDTLESISNEIVIVAT | |
| PDGGSILKISNKYHTIGDHEVEAEQVEASKEMGETLLRAVERYLLAHSHAYN | |
| N | |
| Bet v 1 clone (“3039”): | |
| GVFNVETETASVIPAARLFLAFILDGDTLFPQVAPEAISSVSNISGNGGPGTI | (SEQ ID NO 36) |
| KKISFPEGLPFNYVKDRVDGVDHTNFKYNYSVIGGGPIGDTLESISNEIVIVA | |
| TPDGGSILKISNKYHTIGDHEVEAEQVEASKEMGETLLRAVEAYLLAHSHAY | |
| NN | |
| Bet v 1 clone (“3040”): | |
| GVFNVETETPSVIPAARLFKAFILDGDNLFPKVAPPAISSVSNISGNGGPGTI | (SEQ ID NO 37) |
| KKISFPEGLPFNYVKDRVDSVDHTKFKYNYSVIGGGPIGDTLESISNEIVIVAT | |
| PDGGSILKISNKYHTIGDHEVEAEQVEASKEMGETLLRAVETYLLAHSHAYN | |
| N | |
| Bet v 1 clone “3041”): | |
| GVFNVETETPSVIPAARLFKAFILDGDNLFPKVAPPAISSVSNISGNGGPGTI | (SEQ ID NO 38) |
| KKISFPEGLPFNYVKDRVDRVDHTKFKYNYSVIGGGPIGDTLESISNEIVIVAT | |
| PDGGSILKISNKYHTIGDHEVEAEQVEASKEMGETLLRAVERYLLAHSHAYN | |
| N | |
| Bet v 1 clone (“3042”): | |
| GVFNVETETPSVIPAARLFKAFILDGDNLFPKVAPPAISSVSNISGNGGPGTI | (SEQ ID NO 39) |
| KKISFPEGLPFNYVKDRVDRVDHTKFKYNYSVIGGGPIGDTLESISNEIVIVAT | |
| PDGGSILKISNKYHTIGDHEVEAEQVEASKEMGETLLRAVERYLLAHSHAYN | |
| N | |
| Bet v 1 clone (“3043”): | |
| GVFNVETETPSVIPAARLFLAFILDGDTLFPQVAPKAISSVSNISGNGGPGTI | (SEQ ID NO 40) |
| KKISFPEGLPFNYVKDRVDRVDHTKFKYNYSVIDGGPIGDTLESISNEIVIVAT | |
| PDGGSILKISNKYHTIGDHEVEAEQVEASKEMGETLLRAVEPYLLAHSHAYN | |
| N | |
| Bet v 1 clone (“3044”): | |
| GVFNVETETPSVIPAARLFLAFILDGDTLFPQVAPKAISSVSNISGNGGPGTI | (SEQ ID NO 41) |
| KKISFPEGLPFNYVKDRVDGVDHTKFKYNYSVIGGGPIGDTLESISNEIVIVA | |
| TPDGGSILKISNKYHTIGDHEVEAEQVEASKEMGETLLRAVETYLLAHSHAY | |
| NN | |
| Bet v 1 clone (“3045”): | |
| GVFNVETETPSVIPAARLFMAFILDGDNLFPKVAPPAISSVSNISGNGGPGTI | (SEQ ID NO 42) |
| KKISFPEGLPFNYVKDRVDGVDHTKFKYNYSVIDGGPIGDTLESISNEIVIVAT | |
| PDGGSILKISNKYHTIGDHEVEAEQVEASKEMGETLLRAVEGYLLAHSHAYN | |
| N | |
| Bet v 1 (3010): | |
| Y5V, N28T, K32Q, E45S, K97S, P108G, +160N, E605, T10N, K103V, K65N, | |
| K129N, D125Y, E42S, S149T. | |
| GVFNvETETnSVIPAARLFKAFILDGDtLFPqVAPQAISSVsNIsGNGGPGTIKK | (SEQ ID NO 43) |
| ISFPsGLPFnYVKDRVDEVDHTNFKYNYSVIEGGPIGDTLEsISNEIvIVATgDG | |
| GSILKISNKYHTKGyHEVflAEQVKASKEMGETLLRAVEtYLLAHSDAYNn | |
| Bet v 1 (3011): | |
| Y5V, N28T, K32Q, E455, K97S, P108G, +160N, E6OS, T10N, K103V, K65N, | |
| K129N, D125Y, E425, 5149T, K134E, N47K, T77N, V2L. | |
| GIFNvETETnSVIPAARLFKAFILDGDtLFPqVAPQAISSVsNIsGkGGPGTIKKI | (SEQ ID NO 44) |
| SFPsGLPFnYVKDRVDEVDHnNFKYNYSVIEGGPIGDTLEsISNEIvIVATgDG | |
| GSILKISNKYHTKGYHEVnAEQVeASKEMGETLLRAVEtYLLAHSDAYNn | |
| Bet v 1 (3012): | |
| Y5V, N28T, K320, E45S, K97S, P108G, +160N, E60S, T10N, K103V, K65N, | |
| K129N, 0125Y, E42S, S149T, K134E, N47K, T77N, V2L, E87A, A16G, | |
| Q36N, E73S, D93S. | |
| GIFNvETETnSVIPAgRLFKAFILDGDtLFPqVAPnAISSVsNISGkGGPGTIKKIS | (SEQ ID NO 45) |
| FPsGLpFnyVKDRVDsVDHnNFKYNYSVIaGGPIGsTLEsISNEIvIVATgDGGS | |
| ILKISNKYHTKGyHEVnAEQVeASKEMGETLLRAVEtYLLAHSDAYNn | |

Diagnostic Assay

Furthermore, the recombinant mutant allergens according to the invention have diagnostic possibilities and advantages. Prior art allergy vaccines are based on extracts of the naturally occurring allergen source, and thus represent a wide variety of isoforms. The allergic individual has initially been sensitised and has IgE to one or some of the isoforms present. Some of the isoforms may be relevant with respect to the allergic reactions of the allergic individual due to homology and subsequent cross-reactivity with the isoform to which the individual is allergic, whereas other isoforms may be irrelevant as they do not harbour any of the IgE binding epitopes to which the allergic individual has specific IgE. Due to this heterogeneity of the specificities of the IgE population, some isoforms may therefore be safe to administer, i.e. they do not result in an allergic response via IgE, whereas other isoforms may be harmful causing undesirable side-effects.

Thus, the mutants of the invention and the compositions of the invention intended to be administered therapeutically may also be used for an in vivo or in vitro diagnostic assay to monitor the relevance, safety or outcome of a treatment with such mutants or compositions. Diagnostic samples to be applied include body samples, such as blood or sera.

Thus, the invention also relates to a diagnostic assay for assessing relevance, safety or outcome of therapy of a subject using a recombinant mutant allergen according to the invention or a composition according to the invention, wherein an IgE containing sample of the subject is mixed with said mutant or said composition and assessed for the level of reactivity between the IgE in said sample and said mutant. The assessing of the level of reactivity between the IgE in the sample and the mutant may be carried out using any known immunoassay.

The present invention is further illustrated by the following non-limiting examples.

### EXAMPLES

### Example 1

This Example describes characterisation of recombinant mutant Bet v 1 mutant allergens with diminished IgE-binding affinity. The specific mutant allergens are also disclosed in PCT/DK 01/00764. The following represents an illustrating example of how to prepare mutants according to the present invention.

Identification of Common Epitopes within Fagales Pollen Allergens

The major birch pollen allergen Bet v 1 shows about 90% amino acid sequence identity with major allergens from pollens of taxonomically related trees, i.e Fagales (for instance hazel and hornbeam) and birch pollen allergic patients often show clinical symptoms of allergic cross-reactivity towards these Bet v 1 homologous proteins.

Bet v 1 also shows about 50-60% sequence identity with allergic proteins present in certain fruits (for instance apple and cherry) and vegetables (for instance celery and carrot) and there are clinical evidence for allergic cross-reactivity between Bet v 1 and these food related proteins.

In addition, Bet v 1 shares significant sequence identity (20-40% ) with a group of plant proteins called pathogenesis-related proteins (PR-10), however there are no reports of allergic cross-reactivity towards these PR-10 proteins.

Molecular modelling suggests that the structures of Fagales and food allergens and PR-10 proteins are close to being identical with the Bet v 1 structure.

The structural basis for allergic Bet v 1 cross-reactivity was reported in (Gajhede et al 1996, ref. 17). Thus, any IgE recognising epitopes on Bet v 1 would be able to cross-react and bind to other Fagales major pollen allergens and give rise to allergic symptoms.

Selection of Amino Acid Residues for Site-Directed Mutagenesis

Amino acid residues for site-directed mutagenesis were selected among surface exposed residues present in Bet v 1. The relative orientation and percentage of solvent-exposure of each amino acid residue was calculated based on their atomic coordinates. Residues having a low degree of solvent exposure (<20% ) were not regarded relevant for mutagenesis due to the possible disruption of the structure or lack of antibody interaction. The remaining residues were ranked according to their degree of solvent-exposure.

Sequence Alignment

Sequences homologous to the query sequence (Bet v 1 No. 2801, WHO IUIS Nomenclature Subcommittee on Allergens) were derived from GenBank and EMBL sequence databases by a BLAST search (Altschul et al., ref. 18). All sequences with BLAST reported probabilities less than 0.1 were taken into consideration and one list were constructed containing a non-redundant list of homologous sequences. These were aligned by CLUSTAL W (Higgins et al., ref. 19) and the percentage identity were calculated for each position in the sequence considering the complete list or taxonomically related species only. A total of 122 sequences were homologous to Bet v 1 No. 2801 of which 57 sequences originates from taxonomically related species.

Cloning of the Gene Encoding Bet v 1

RNA was prepared from Betula verrucosa pollen (Allergon, Sweden) by phenol extraction and LiCl precipitation. Oligo(dT)-cellulose affinity chromatography was performed batch-wise in Eppendorph tubes, and double-stranded cDNA was synthesised using a commercially available kit (Amersham). DNA encoding Bet v 1 was amplified by PCR and cloned. In brief, PCR was performed using cDNA as template, and primers designed to match the sequence of the cDNA in positions corresponding to the amino terminus of Bet v 1 and the 3′-untranslated region, respectively. The primers were extended in the 5′-ends to accommodate restriction sites (Ncol and Hindlll) for directional cloning into pKK233-2.

Subcloning into pMAL-c

The gene encoding Bet v 1 was subsequently subcloned into the maltose binding protein fusion vector pMAL-c (New England Biolabs). The gene was amplified by PCR and subcloned in frame with malE to generate maltose binding protein (MBP)-Bet v 1 protein fusion operons in which MBP and Bet v 1 were separated by a factor Xₐ protease clevage site positioned to restore the authentic aminoterminal sequence of Bet v 1 upon cleavage, as described in ref. 15. In brief, PCR was performed using pKK233-3 with Bet v 1 inserted as template and primers corresponding to the amino- and carboxyterminus of the protein, respectively. The promoter proximal primer was extended in the 5′-end to accommodate 4 codons encoding an in frame factor Xₐ protease cleavage site. Both primers were furthermore extended in the 5′-ends to accommodate restriction sites (Kpnl) for cloning. The Bet v 1 encoding genes were subcloned using 20 cycles of PCR to reduce the frequency of PCR artefacts.

In Vitro Mutagenesis

In vitro mutagenesis was performed by PCR using recombinant pMAL-c with Bet v 1 inserted as template. Each mutant Bet v 1 gene was generated by 3 PCR reactions using 4 primers. The following examples of mutants are according to prior art PCT/DK 01/00764. Mutants according to the invention can be prepared and assayed in a similar fashion.

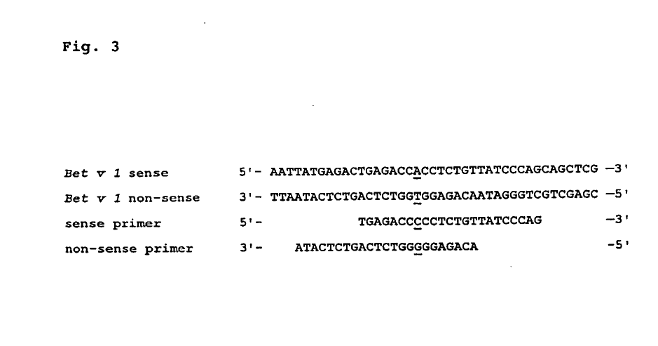

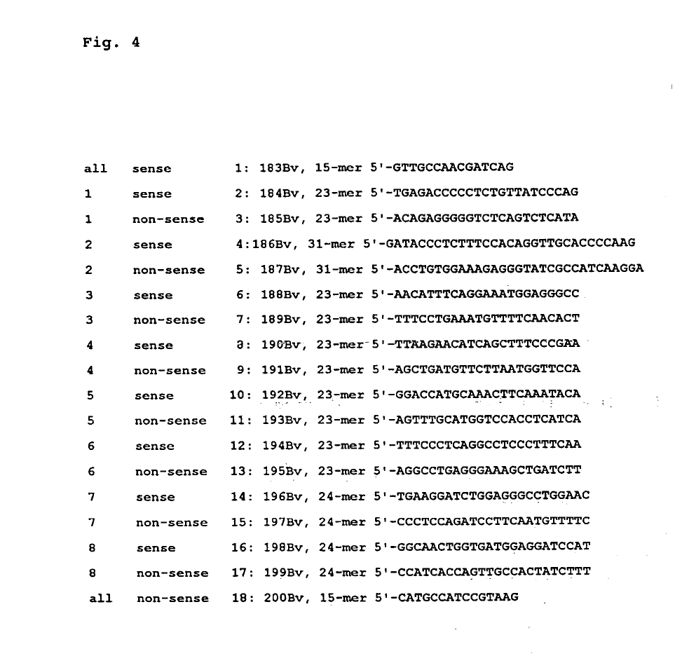

Two mutation-specific oligonucleotide primers were synthesised accommodating each mutation, one for each DNA strand, see FIGS. 3 and 4. Using the mutated nucleotide(s) as starting point both primers were extended 7 nucleotides in the 5′-end and 15 nucleotides in the 3′-end. The extending nucleotides were identical in sequence to the Bet v 1 gene in the actual region.

Two generally applicable primers (denoted “all-sense” and “all non-sense” in FIG. 4) were furthermore synthesised and used for all mutants. These primers were 15 nucleotides in length and correspond in sequence to regions of the pMAL-c vector approximately 1 kilobase upstream and downstream from the Bet v 1. The sequence of the upstream primer is derived from the sense strand and the sequence of the downstream primer is derived from the non-sense strand, see FIG. 4.

Two independent PCR reactions were performed essentially according to standard procedures (Saiki et al 1988, ref. 20) with the exception that only 20 temperature cycles were performed in order to reduce the frequency of PCR artefacts. Each PCR reaction used pMAL-c with Bet v 1 inserted as template and one mutation-specific and one generally applicable primer in meaningful combinations.

Introduction of the four amino acid substitutions (Asn28Thr, Lys32Gln, Glu45Ser, Pro108Gly) in the mutant were performed like described above in a step by step process. First the Glu45Ser mutation then the Pro108Gly mutation and last the Asn28Thr, and Lys32Gln mutations were introduced using pMAL-c with inserted Bet v 1 No. 2801, Bet v 1 (Glu45Ser), Bet v 1 (Glu45Ser, Pro108Gly) as templates, respectively.

The PCR products were purified by agarose gel electrophoresis and electro-elution followed by ethanol precipitation. A third PCR reaction was performed using the combined PCR products from the first two PCR reactions as template and both generally applicable primers. Again, 20 cycles of standard PCR were used. The PCR product was purified by agarose gel electrophoresis and electro-elution followed by ethanol precipitation, cut with restriction enzymes (BslWI/EcoRI), and ligated directionally into pMAL-c with Bet v 1 inserted restricted with the same enzymes.

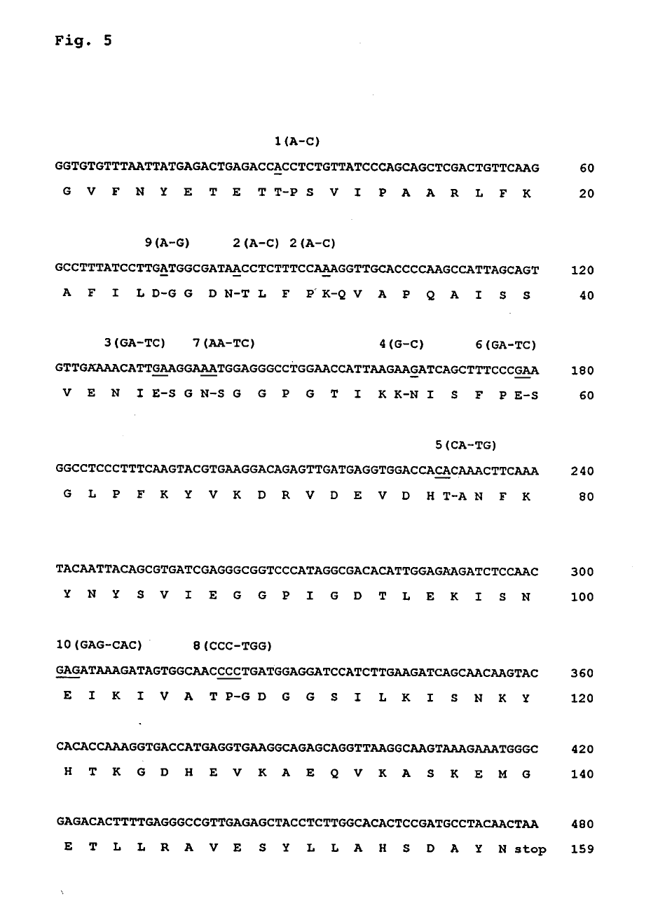

FIG. 5 shows an overview of all 9 Bet v 1 mutations, which are as follows

Thr10Pro, Asp25Gly, Asn28Thr +Lys32Gln, Glu45Ser, Asn47Ser, Lys55Asn, Glu60Ser, Thr77Ala and Pro108Gly. An additional mutant with four mutations was also prepared (Asn28Thr, Lys32Gln, Glu45Ser, Pro108Gly). Of these, five mutants were selected for further testing: Asn28Thr+Lys32Gln, Glu45Ser, Glu60Ser, Pro108Gly and the Asn28Thr, Lys32Gln, Glu45Ser, Pro108Gly mutant.

Nucleotide Sequencing

Determination of the nucleotide sequence of the Bet v 1 encoding gene was performed before and after subcloning, and following in vitro mutagenesis, respectively.

Plasmid DNA's from 10 ml of bacterial culture grown to saturation overnight in LB medium supplemented with 0.1 g/l ampicillin were purified on Qiagen-tip 20 columns and sequenced using the Sequenase version 2.0 DNA sequencing kit (USB) following the recommendations of the suppliers.

Expression and Purification of Recombinant Bet v 1 and Mutants

Recombinant Bet v 1 (Bet v 1 No. 2801 and mutants) were over-expressed in Escherichia coli DH 5a fused to maltose-binding protein and purified as described in ref. 15. Briefly, recombinant E.coli cells were grown at 37° C. to an optical density of 1.0 at 436 nm, whereupon expression of the Bet v 1 fusion protein was induced by addition of IPTG. Cells were harvested by centrifugation 3 hours post-induction, re-suspended in lysis buffer and broken by sonication. After sonication and additional centrifugation, recombinant fusion protein was isolated by amylose affinity chromatography and subsequently cleaved by incubation with Factor Xa (ref. 15). After F Xa cleavage, recombinant Bet v 1 was isolated by gelfiltration and if found necessary, subjected to another round of amylose affinity chromatography in order to remove trace amounts of maltose-binding protein.

Purified recombinant Bet v 1 was concentrated by ultrafiltration to about 5 mg/ml and stored at 4° C. The final yields of the purified recombinant Bet v 1 preparations were between 2-5 mg per litre E. coli cell culture.

The purified recombinant Bet v 1 preparations appeared as single bands after silver-stained SDS-polyacrylamide electrophoresis with an apparent molecular weight of 17.5 kDa. N-terminal sequencing showed the expected sequences as derived from the cDNA nucleotide sequences and quantitative amino acid analysis showed the expected amino acid compositions.

We have previously shown (ref. 15) that recombinant Bet v 1 No. 2801 is immunochemically indistinguishable from naturally occurring Bet v 1.

Immunoelectrophoresis using Rabbit Polyclonal Antibodies

The seven mutant Bet v 1 were produced as recombinant Bet v 1 proteins and purified as described above and tested for their reactivity towards polyclonal rabbit antibodies raised against Bet v 1 isolated from birch pollen. When analysed by immunoelectrophoresis (rocket-line immunoelectrophoresis) under native conditions, the rabbit antibodies were able to precipitate all mutants, indicating that the mutants had conserved α-carbon backbone tertiary structure.

In order to analyse the effect on human polyclonal IgE-response, the mutants Glu45Ser, Pro108Gly, Asn28Thr+Lys32Gln and Glu60Ser were selected for further analysis.

Bet v 1 Glu45Ser Mutant

Glutamic acid in position 45 show a high degree of solvent-exposure (40% ). A serine residue was found to occupy position 45 in some of the Bet v 1 homologous PR-10 proteins arguing for that glutamic acid can be replaced by serine without distortion of the α-carbon backbone tertiary structure. In addition, as none of the known Fagales allergen sequences have serine in position 45, the substitution of glutamic acid with serine gives rise to a non-naturally occurring Bet v 1 molecule.

T Cell Proliferation Assay using Recombinant Glu45Ser Bet v 1 Mutant

The analysis was carried out as described in Spangfort et al 1996a. It was found that recombinant Bet v 1 Glu45Ser mutant was able to induce proliferation in T cell lines from three different birch pollen allergic patients with stimulation indices similar to recombinant and naturally occurring.

Crystallisation and Structural Determination of Recombinant Glu45Ser Bet v 1

Crystals of recombinant Glu45Ser Bet v 1 were grown by vapour diffusion at 25° C., essentially as described in (Spangfort et al 1996b, ref. 21). Glu45Ser Bet v 1, at a concentration of 5 mg/ml, was mixed with an equal volume of 2.0 M ammonium sulphate, 0.1 M sodium citrate, 1% (v/v) dioxane, pH 6.0 and equilibrated against 100× volume of 2.0 M ammonium sulfate, 0.1 M sodium citrate, 1% (v/v) dioxane, pH 6.0. After 24 hours of equilibration, crystal growth was induced by applying the seeding technique described in ref. 21, using crystals of recombinant wild-type Bet v 1 as a source of seeds.

After about 2 months, crystals were harvested and analysed using X-rays generated from a Rigaku rotating anode as described in ref. 21 and the structure was solved using molecular replacement.

Structure of Bet v 1 Glu45Ser Mutant

The structural effect of the mutation was addressed by growing three-dimensional Bet v 1 Glu45Ser protein crystals diffracting to 3.0 Å resolution when analysed by X-rays generated from a rotating anode. The substitution of glutamic acid to serine in position 45 was verified by the Bet v 1 Glu45Ser structure electron density map which also showed that the overall α-carbon backbone tertiary structure is preserved.

IgE-Binding Properties of Bet v 1 Glu45Ser Mutant

The IgE-binding properties of Bet v 1 Glu45Ser mutant was compared with recombinant Bet v 1 in a fluid-phase IgE-inhibition assay using a pool of serum IgE derived from birch allergic patients.

Recombinant Bet v 1 no. 2801 was biotinylated at a molar ratio of 1:5 (Bet v 1 no. 2801:biotin). The inhibition assay was performed as follows: a serum sample (25 μl) was incubated with solid phase anti IgE, washed, re-suspended and further incubated with a mixture of biotinylated Bet v 1 no. 2801 (3.4 nM) and a given mutant (0-28.6 nM). The amount of biotinylated Bet v 1 no. 2801 bound to the solid phase was estimated from the measured RLU after incubation with acridinium ester labelled streptavidin. The degree of inhibition was calculated as the ratio between the RLU's obtained using buffer and mutant as inhibitor.

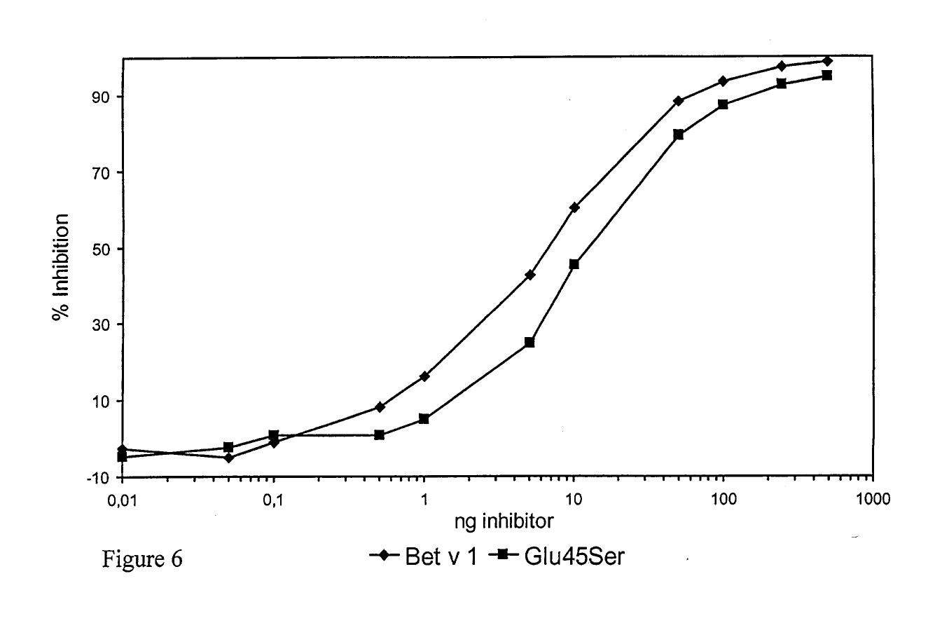

FIG. 6 shows the inhibition of the binding of biotinylated recombinant Bet v 1 to serum IgE from a pool of allergic patients by non-biotinylated Bet v 1 and by Bet v 1 Glu45Ser mutant.

There is a clear difference in the amount of respective recombinant proteins necessary to reach 50% inhibition of the binding to serum IgE present in the serum pool. Recombinant Bet v 1 reaches 50% inhibition at about 6.5 ng whereas the corresponding concentration for Bet v 1 Glu45Ser mutant is about 12 ng. This show that the point mutation introduced in Bet v 1 Glu45Ser mutant lowers the affinity for specific serum IgE by a factor of about 2.

The maximum level of inhibition reached by the Bet v 1 Glu45Ser mutant is clearly lower compared to recombinant Bet v 1. This may indicate that after the Glu45Ser substitution, some of the specific IgE present in the serum pool are unable to recognise the Bet v 1 Glu45Ser mutant.

Bet v 1 Mutant Asn28Thr+Lys32Gln

Aspartate and lysine in positions 28 and 32, respectively show a high degree of solvent-exposure (35% and 50%, respectively). In the structure, aspartate 28 and lysine 32 are located close to each other on the molecular surface and most likely interact via hydrogen bonds. A threonine and a gluatamate residue were found to occupy positions 28 and 32, respectively in some of the Bet v 1 homologous PR-10 proteins arguing for that aspartate and lysine can be replaced with threonine and glutamate, respectively without distortion of the α-carbon backbone tertiary structure. In addition, as none of the naturally occurring isoallergen sequences have threonine and glutamate in positions 28 and 32, respectively, the substitutions gives rise to a non-naturally occurring Bet v 1 molecule.

IgE-Binding Properties of Bet v 1 Mutant Asn28Thr+Lys32Gln

The IgE-binding properties of mutant Asn28Thr+Lys32Gln was compared with recombinant Bet v 1 in a fluid-phase IgE-inhibition assay using the pool of serum IgE derived from birch allergic patients described above.

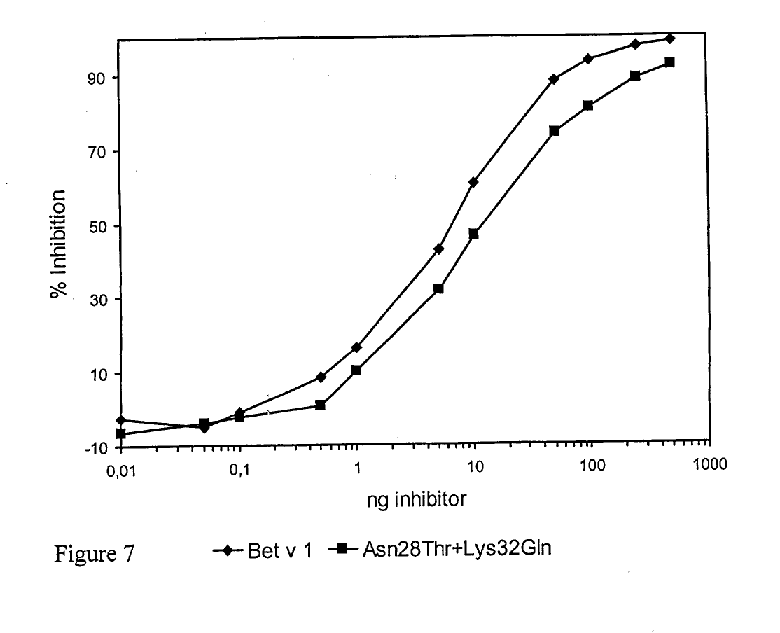

FIG. 7 shows the inhibition of the binding of biotinylated recombinant Bet v 1 to serum IgE from a pool of allergic patients by non-biotinylated Bet v 1 and by Bet v 1 mutant Asn28Thr+Lys32Gln.

There is a clear difference in the amount of respective recombinant proteins necessary to reach 50% inhibition of the binding to serum IgE present in the serum pool. Recombinant Bet v 1 reaches 50% inhibition at about 6.5 ng whereas the corresponding concentration for Bet v 1 mutant Asn28Thr+Lys32Gln is about 12 ng. This show that the point mutations introduced in Bet v 1 mutant Asn28Thr+Lys32Gln lowers the affinity for specific serum IgE by a factor of about 2.

The maximum level of inhibition reached by the Bet v 1 mutant Asn28Thr+Lys32Gln mutant is clearly lower compared to recombinant Bet v 1. This may indicate that after the Asn28Thr+Lys32Gln substitutions, some of the specific IgE present in the serum pool are unable to recognise the Bet v 1 mutant Asn28Thr+Lys32Gln.

Bet v 1 Mutant Pro108Gly

Proline in position 108 shows a high degree of solvent-exposure (60%). A glycine residue was found to occupy position 108 in some of the Bet v 1 homologous PR-10 proteins arguing for that proline can be replaced with glycine without distortion of the α-carbon backbone tertiary structure. In addition, as none of the naturally occurring isoallergen sequences have glycine in position 108, the substitution of proline with glycine gives rise to a non-naturally occurring Bet v 1 molecule.

IgE-Binding Properties of Bet v 1 Pro108Gly Mutant

The IgE-binding properties of Bet v 1 Pro108Gly mutant was compared with recombinant Bet v 1 in a fluid-phase IgE-inhibition assay using the pool of serum IgE derived from birch allergic patients described above.

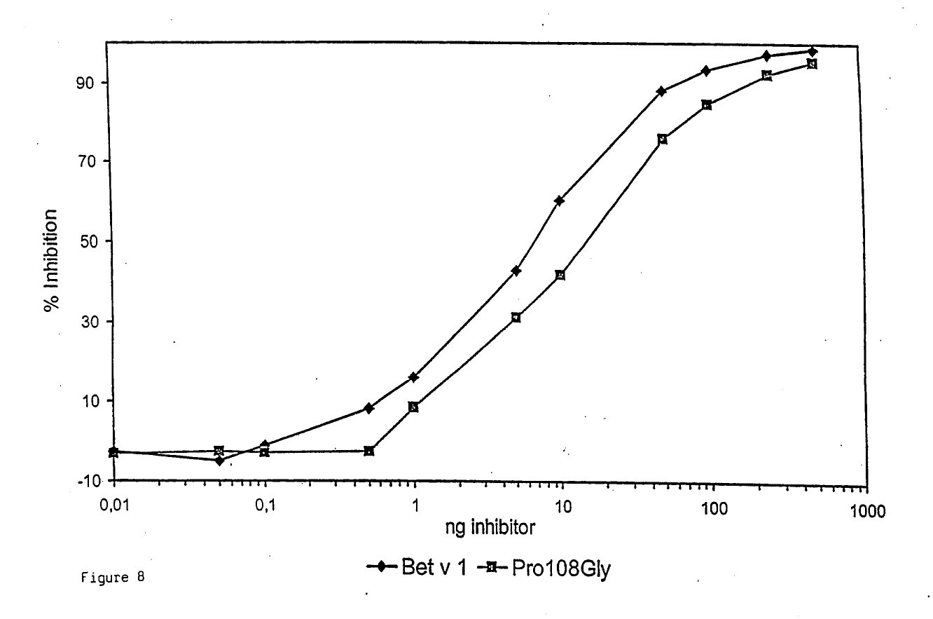

FIG. 8 shows the inhibition of the binding of biotinylated recombinant Bet v 1 to serum IgE from a pool of allergic patients by non-biotinylated Bet v 1 and by Bet v 1 Pro108Gly mutant.

There is a clear difference in the amount of respective recombinant proteins necessary to reach 50% inhibition of the binding to serum IgE present in the serum pool. Recombinant Bet v 1 reaches 50% inhibition at about 6.5 ng whereas the corresponding concentration for Bet v 1 Pro108Gly is 15 ng. This show that the single point mutation introduced in Bet v 1 Pro108Gly lowers the affinity for specific serum IgE by a factor of about 2.

The maximum level of inhibition reached by the Bet v 1 Pro108Gly mutant is somewhat lower compared to recombinant Bet v 1. This may indicate that after the Pro108Gly substitution, some of the specific IgE present in the serum pool are unable to recognise the Bet v 1 Pro108Gly mutant.

Bet v 1 Mutant Glu60Ser Mutant

Glutamic acid in position 60 show a high degree of solvent-exposure (60%). A serine residue was found to occupy position 60 in some of the Bet v 1 homologous PR-10 proteins arguing for that glutamic acid can be replaced with serine without distortion of the α-carbon backbone tertiary structure. In addition, as none of the naturally occurring isoallergen sequences have serine in position 60, the substitution of glutamic acid with serine gives rise to a non-naturally occurring Bet v 1 molecule.

IgE-Binding Properties of Bet v 1 Glu60Ser Mutant

The IgE-binding properties of Bet v 1 Glu60Ser mutant was compared with recombinant Bet v 1 in a fluid-phase IgE-inhibition assay using the pool of serum IgE derived from birch allergic patients described above.

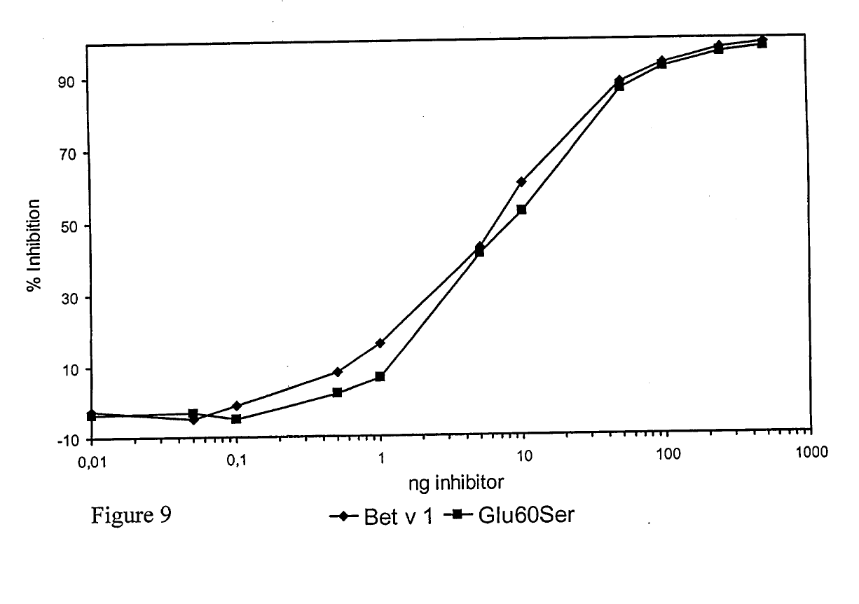

FIG. 9 shows the inhibition of the binding of biotinylated recombinant Bet v 1 to serum IgE from a pool of allergic patients by non-biotinylated Bet v 1 and by Bet v 1 Glu60Ser mutant. In contrast to the Glu45Ser, Pro108Gly and Asn28Thr+Lys32Gln mutants, the substitution glutamic acid 60 to serine, does not shown any significant effect on the IgE-binding properties of.

Structural Analysis of Bet v 1 Glu45Ser, Asn28Thr+Lys32Gln and Pro108Gly Mutant

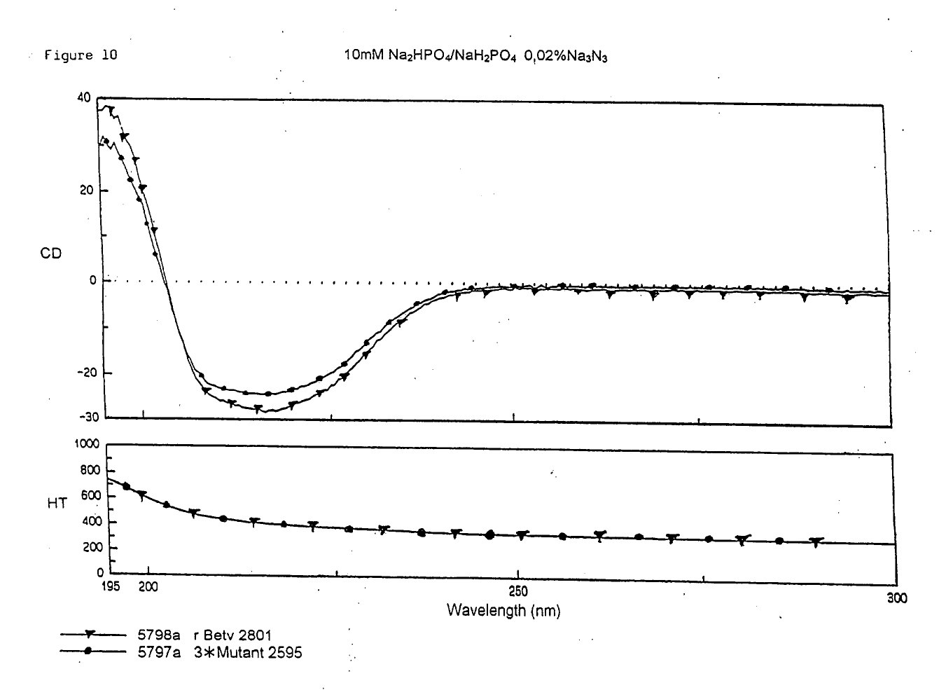

The structural integrity of the purified recombinant protein was analysed by circular dichroism (CD) spectroscopy. FIG. 10 shows the CD spectra of recombinant mutant and recombinant naturally occurring protein, recorded at close to equal concentrations. The overlap in peak amplitudes and positions in the CD spectra from the two recombinant proteins shows that the two preparations contain equal amounts of secondary structures strongly suggesting that the α-carbon backbone tertiary structure is not affected by the introduced amino acid substitutions.

IgE-Binding Properties of Bet v 1 Glu45Ser, Asn28Thr+Lys32Gln and Pro108Gly Mutant

The IgE-binding properties of the mutant was compared with recombinant Bet v 1 in a fluid-phase IgE-inhibition assay using the pool of serum IgE derived from birch allergic patients described above.

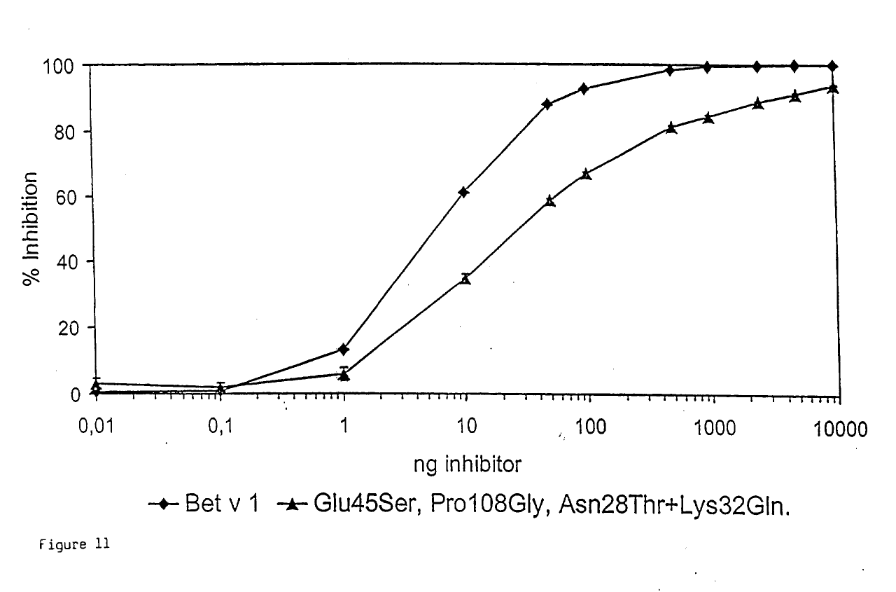

FIG. 11 shows the inhibition of the binding of biotinylated recombinant Bet v 1 to serum IgE from a pool of allergic patients by non-biotinylated Bet v 1 and by the Bet v 1 mutant. In contrast to the single mutants described above, the inhibition curve of the mutant is no longer parallel relative to recombinant. This shows that the substitutions introduced in the mutant have changed the IgE-binding properties and epitope profile compared to recombinant. The lack of parallellity makes it difficult to quantify the decrease of the mutants affinity for specific serum IgE.

Recombinant Bet v 1 reaches 50% inhibition at about 6 ng whereas the corresponding concentration for Bet v 1 (Asn28Thr, Lys32Gln, Glu45Ser, Pro108Gly) mutant is 30 ng, i.e a decrease in affinity by a factor 5. However, in order to reach 80% inhibition the corresponding values are 20 ng and 400 ng, respectively, i.e a decrease by a factor 20.

T Cell Proliferation Assay Using the Recombinant Bet v 1 Glu45Ser, Asn28Thr+Lys32Gln and Pro108Gly Mutant

The analysis was carried out as described in ref. 15. It was found that recombinant Bet v 1 mutant was able to induce proliferation in T cell lines from three different birch pollen allergic patients with stimulation indices similar to recombinant and naturally occurring. This suggests that the mutant can initiate the cellular immune response necessary for antibody production.

### Example 2

In Vitro Mutagenesis of Mutants According to the Present Invention

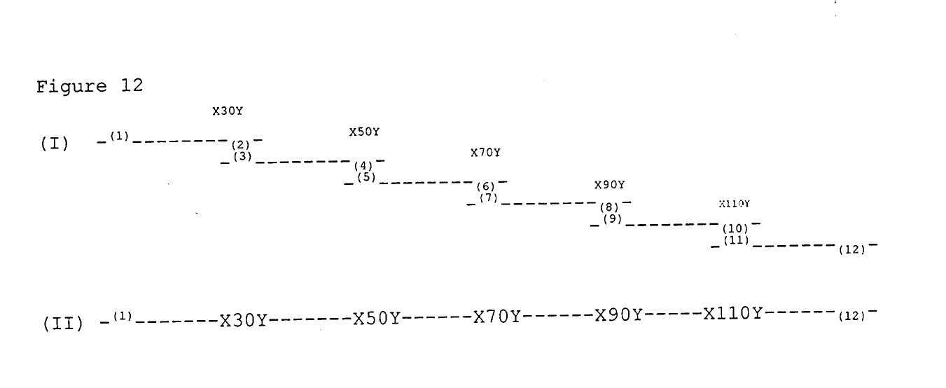

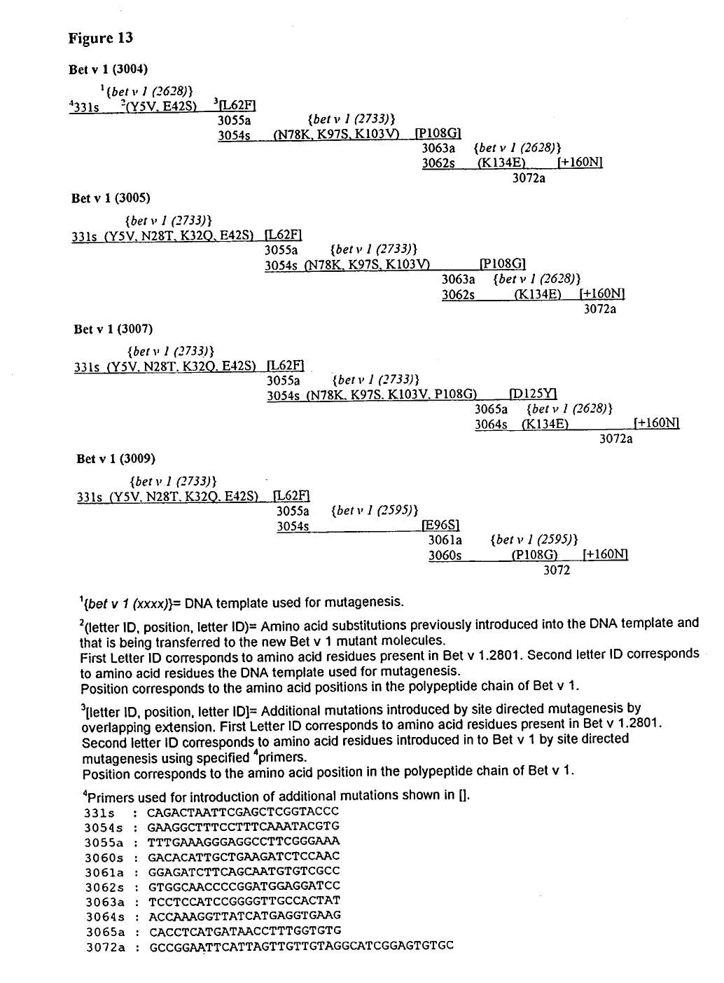

In vitro mutagenesis was performed by PCR using recombinant pMAL-c with Bet v 1 inserted as template. Preparation of recombinant mutant allergens included two PCR steps; step I and II. First, each single mutation (or several mutations if located closely together in the DNA sequence) was introduced into sequential DNA sequences of Bet v 1.2801 derivatives i.e. Bet v 1 (2595) or Bet v 1 (2628) or Bet v 1 (2733) using sense and anti-sense mutation-specific oligonucleotide primers accommodating each mutation(s) along with sense and anti-sense oligonucleotide primers accommodating either upstream or downstream neighbour mutations or the N-terminus/C-terminus of Bet v 1, respectively as schematically illustrated in FIG. 12 (I). Secondly, PCR products from PCR reaction I were purified, mixed and used as templates for an additional PCR reaction (II) with oligonucleotide primers accommodating the N-terminus and C-terminus of Bet v 1 as schematically illustrated in FIG. 13 (II). The PCR products were purified by agarose gel electrophoresis and PCR gel purification (Life Techhnologies) followed by ethanol precipitation, cut with restriction enzymes (SacI/EcoRI) or (SacI/XbaI), and ligated directionally into pMAL-c restricted with the same enzymes.

FIG. 13 shows synthesised oligonucleotide primers and schematically illustrations for the construction of Bet v 1 mutants. The following Bet v 1 mutants were cloned and sequenced (sequencing of nucleic acid molecules is described in Example 1):

| | |
|---|---|
| Bet v 1 (3004): | |
| GVFNvETETTSVIPAARLFKAFILDGDNLFPKVAPQAISSVsNIEGNGGPGTIK | (SEQ ID NO 6) |
| KISFPEGfPFKYVKDRVDEVDHTkFKYNYSVIEGGPIGDTLEsISNEIvIVATPD | |
| GGSILKISNKYHTKGDHEVKAEQVeASKEMGETLLRAVESYLLAHSDAYNn | |
| Bet v 1 (3005): | |
| GVFNvETETTSVIPAARLFKAFILDGDtLFPqVAPQAISSVENIsGNGGPGTIK | (SEQ ID NO 7) |
| KISFPEGfPFKYVKDRVDEVDHTkFKYNYSVIEGGPIGDTLEsISNEIvIVATPD | |
| GGSILKISNKYHTKGDHEVKAEQVeASKEMGETLLRAVESYLLAHSDAYNn | |
| Bet v 1 (3007): | |
| GVFNvETETTSVIPAARLFKAFILDGDtLFPqVAPQAISSVENIsGNGGPGTIK | (SEQ ID NO 8) |
| KISFPEGfPFKYVKDRVDEVDHTkFKYNYSVIEGGPIGDTLEsISNEIvIVATgD | |
| GGSILKISNKYHTKGyHEVKAEQVeASKEMGETLLRAVESYLLAHSDAYNn | |
| Bet v 1 (3009): | |
| GVFNvETETTSVIPAARLFKAFILDGDtLFPqVAPQAISSVENIsGNGGPGTIK | (SEQ ID NO 9) |
| KISFPEGfPFKYVKDRVDEVDHTNFKYNYSVIEGGPIGDTLsKISNEIKIVATg | |
| DGGSILKISNKYHTKGDHEVKAEQVKASKEMGETLLRAVESYLLAHSDAYN | |
| n | |
| Bet v 1 (3006): | |
| GVFNvETETTSVIPAARLFKAFILDGDtLFPqVAPQAISSVENIsGNGGPGTIK | (SEQ ID NO 10) |
| KISFPEGfPFKYVKDRVDEVDHTkFKYNYSVIEGGPIGDTLEsISNEIvIVATPD | |
| GGSILKISNKYHTKGDHEVKAEQVeASKEMGETLLRAVESYLLAHSDAYgn | |
| Bet v 1 (3008): | |
| GVFNvETETTSVIPAARLFKAFILDGDtLFPkVAPQAISSVENIsGNGGPGTIK | (SEQ ID NO 11) |
| KISFPEGfPFKYVKDRVDsVDHTNFKYNYSVIEGGPIGDTLsKISNEIKIVATg | |
| DGGSILKISNKYHTKGyHEVKAEQVKASKEMGETLLRAVESYLLAHSDAYgn | |

Further Mutants Prepared According to the Present Invention:

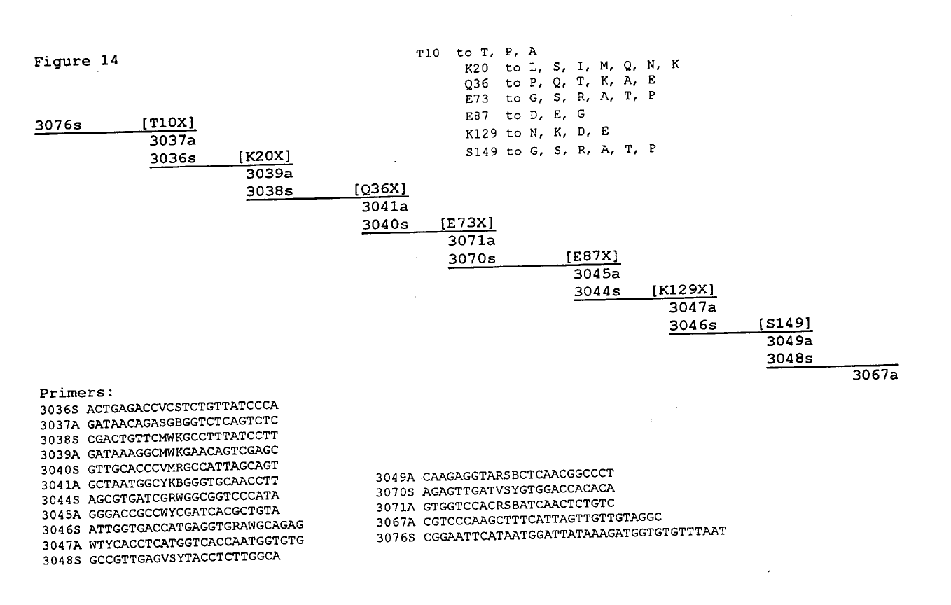

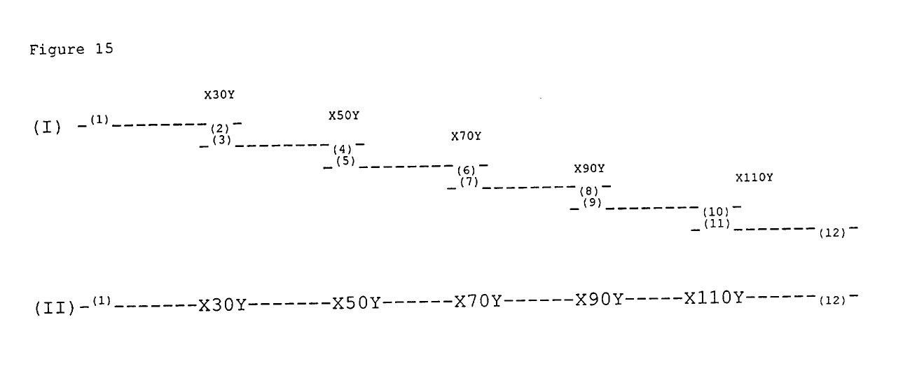

Introduction of multiple point mutations into Bet v 1 may potentially destabilize the α-carbon backbone folding-pattern of the molecule. Introduction of random amino acid substitutions increases the chances of generating stable mutant Bet v 1 molecules. We therefore generated a Bet v 1 mutant library containing Bet v 1 mutants with 17-20 point mutations of which amino acid substitutions were randomly substituted in 7 positions. The library contained hundreds of different clones. Fifteen Bet v 1 mutants named Bet v 1 (3031) to (3045) were obtained from this Bet v 1 mutant library generated using degenerated oligonucleotide primers. These primers accommodated random substitution of amino acid residues in the positions T10, K20, Q36, E73, E87, K129 and S149 of Bet v 1 (FIGS. 14 and 15). These positions were non-overlapping with point mutations already introduced into Bet v 1 (3002) and Bet v 1 (2595) that were used as DNA templates for the site directed mutagenesis PCR reactions illustrated in FIG. 15.

The cloning procedure was the same as illustrated in FIG. 12 except that the primers used in the first PCR round were degenerated in certain positions as indicated in FIG. 15 by letters other than G, C, T or A. Use of other letters than G, C, T or A indicates that the primers contain several different nucleotides in these positions. Eight PCR products spanning the Bet v 1 gene were produced and purified in the first PCR round and then assembled using end-primers (3076s and 3067a) in a second PCR reaction where the eight PCR products from the first PCR round were used as a template.

The Bet v 1 mutants 3031 to 3045 were DNA sequenced as described for the Bet v 1 3004, 3005, 3007 and 3007 mutants in order to verify the number and nature of the introduced point mutations:

Bet v 1 clone (“3031“):

(SEQ ID NO 28)

GVFNVETETASVIPAARLFNAFILDGDTLFPQVAPQAISSVSNISGNGGP
GTIKKISFPEGLPFNYVKDRVDSVDHTNFKYNYSVIEGGPIGDTLESISN
EIVIVATPDGGSILKISNKYHTIGDHEVEAEQVEASKEMGETLLRAVEPY
LLAHSHAYNN
Bet v 1 clone (“3032”):

(SEQ ID NO 29)

GVFNVETETASVIPAARLFLAFILDGDTLFPQVAPPAISSVSNISGNGGP
GTIKKISFPEGLPFNYVKDRVDPVDHTKFKYNYSVIGGGPIGDTLESISN
EIVIVATPDGGSILKISNKYHTIGDHEVEAEQVEASKEMGETLLRAVEGY
LLAHSHAYNN
Bet v 1 clone (“3033”):

(SEQ ID NO 30)

GVFNVETETPSVIPAARLFHAFILDGDTLFPQVAPKAISSVSNISGNGGP
GTIKKISFPEGLPFNYVKDRVDRVDHTKFKYNYSVIEGGPIGDTLESISN
EIVIVATPDGGSILKISNKYHTIGDHEVEAEQVEASKEMGETLLRAVEGY
LLAHSHAYNN
Bet v 1 clone (“3034”):

(SEQ ID NO 31)

GVFNVETETTSVIPAARLFHAFILDGDNLFPKVAPPAISSVSNISGNGGP
GTIKKISFPEGLPFNYVKDRVDSVDHTKFKYNYSVIGGGPIGDTLESISN
EIVIVATPDGGSILKISNKYHTIGDHEVEAEQVEASKEMGETLLRAVERY
LLAHSHAYNN
Bet v 1 clone (“3035”):

(SEQ ID NO 32)

GVFNVETETPSVIPAARLFMAFILDGDTLFPQVAPPAISSVSNISGNGGP
GTIKKISFPEGLPFNYVKDRVDSVDHTNFKYNYSVIGGGPIGDTLESISN
EIVIVATPDGGSILKISNKYHTIGDHEVEAEQVEASKEMGETLLRAVEAY
LLAHSHAYNN
Bet v 1 clone (“3036”):

(SEQ ID NO 33)

GVFNVETETPSVIPAARLFLAFILDGDNLFPKVAPPAISSVSNISGNGGP
GTIKKISFPEGLPFNYVKDRVDTVDHTKFKYNYSVIGGGPIGDTLESISN
EIVIVATPDGGSILKISNKYHTIGDHEVEAEQVEASKEMGETLLRAVERY
LLAHSHAYNN
Bet v I clone (“3037”):

(SEQ ID NO 34)

GVFNVETETPSVIPAARLFQAFILDGDNLFPKVAPPAISSVSNISGNGGP
GTIKKISFPEGLPFNYVKDRVDSVDHTNFKYNYSVIGGGPIGDTLESISN
EIVIVATPDGGSILKISNKYHTIGDHEVEAEQVEASKEMGETLLRAVEPY
LLAHSHAYNN
Bet v 1 clone (“3038”):

(SEQ ID NO 35)

GVFNVETETASVIPAARLFLAFILDGDNLFPKVAPPAISSVSNISGNGGP
GTIKKISFPEGLPFNYVKDRVDGVDHTKFKYNYSVIDGGPIGDTLESISN
EIVIVATPDGGSILKISNKYHTIGDHEVEAEQVEASKEMGETLLRAVERY
LLAHSHAYNN
Bet v 1 clone (“3039”):

(SEQ ID NO 36)

GVFNVETETASVIPAARLFLAFILDGDTLFPQVAPEAISSVSNISGNGGP
GTIKKISFPEGLPFNYVKDRVDGVDHTNFKYNYSVIGGGPIGDTLESISN
EIVIVATPDGGSILKISNKYHTIGDHEVEAEQVEASKEMGETLLRAVEAY
LLAHSHAYNN
Bet v I clone (“3040”):

(SEQ ID NO 37)

GVFNVETETPSVIPAARLFKAFILDGDNLFPKVAPPAISSVSNISGNGGP
GTIKKISFPEGLPFNYVKDRVDSVDHTKFKYNYSVIGGGPIGDTLESISN
EIVIVATPDGGSILKISNKYHTIGDHEVEAEQVEASKEMGETLLRAVETY
LLAHSHAYNN
Bet v 1 clone “3041”):

(SEQ ID NO 38)

GVFNVETETPSVIPAARLFKAFILDGDNLFPKVAPPAISSVSNISGNGGP
GTIKKISFPEGLPFNYVKDRVDRVDHTKFKYNYSVIGGGPIGDTLESISN
EIVIVATPDGGSILKISNKYHTIGDHEVEAEQVEASKEMGETLLRAVERY
LLAHSHAYNN
Bet v 1 clone (“3042”):

(SEQ ID NO 39)

GVFNVETETPSVIPAARLFKAFILDGDNLFPKVAPPAISSVSNISGNGGP
GTIKKISFPEGLPFNYVKDRVDRVDHTKFKYNYSVIGGGPIGDILESISN
EIVIVATPDGGSILKISNKYHTIGDHEVEAEQVEASKEMGETLLRAVERY
LLAHSHAYNN
Bet v 1 clone (“3043”):

(SEQ ID NO 40)

GVFNVETETPSVIPAARLFLAFILDGDTLFPQVAPKAISSVSNISGNGGP
GTIKKISFPEGLPFNYVKDRVDRVDHTKFKYNYSVIDGGPIGDTLESISN
EIVIVATPDGGSILKISNKYHTIGDHEVEAEQVEASKEMGETLLRAVEPY
LLAHSHAYNN
Bet v 1 clone (“3044”):

(SEQ ID NO 41)

GVFNVETETPSVIPAARLFLAFILDGDTLFPQVAPKAISSVSNISGNGGP
GTIKKISFPEGLPFNYVKDRVDGVDHTKFKYNYSVIGGGPIGDTLESISN
EIVIVATPDGGSILKISNKYHTIGDHEVEAEQVEASKEMGETLLRAVETY
LLAHSHAYNN
Bet v 1 clone (“3045”):

(SEQ ID NO 42)

GVFNVETETPSVIPAARLFMAFILDGDNLFPKVAPPAISSVSNISGNGGP
GTIKKISFPEGLPFNYVKDRVDGVDHTKFKYNYSVIDGGPIGDTLESISN
EIVIVATPDGGSILKISNKYHTIGDHEVEAEQVEASKEMGETLLRAVEGY
LLAHSHAYNN

### Example 3

Identification and Selection of Amino Acids for Substitution

The parameters of solvent accessibility and conservation degree were used to identify and select surface-exposed amino acids suitable for substitution for the allergens Bet v 1, Der p 2 and Ves v 5.

Solvent Accessibility

Solvent accessibility was calculated using the software InsightII, version 97.0 (MSI) and a probe radius of 1.4 Å (Connolly surface).

Internal cavities were excluded from the analyses by filling with probes using the software PASS (Putative Active Sites with Spheres). Probes on the surface were subsequently removed manually.

Conservation

Bet v 1:

3-D structure is based on accession number Z80104 (1bv1.pdb).

38 other Bet v 1 sequences included in the analysis of conserved residues comprise accession numbers:

P15494=X15877=Z80106, Z80101, AJ002107, Z72429, AJ002108, Z80105, Z80100, Z80103, AJ001555, Z80102, AJ002110, Z72436, P43183=X77271, Z72430, AJ002106, P43178=X77267, P43179=X77268, P43177=X77266, Z72438, P43180=X77269, AJ001551, P43185=X77273, AJ001557, Z72434, AJ001556, Z72433=P43186, AJ001554, X81972, Z72431, P45431=X77200, P43184=X77272, P43176=X77265, S47250, S47251, Z72435, Z72439, Z72437,S47249.

Bet v 1

59 amino acids highly solvent exposed:

K-129, E-60, N-47, K-65, P-108, N-159, D-93, K-123, K-32, D-125, R-145, D-109, T-77, E-127, Q-36, E-131, L-152, E-6, E-96, D-156, P-63, H-76, E-8, K-134, E-45, T-10, V-12, K-20, L-62, S-155, H-126, P-50, N-78, K-119, V-2, L-24, E-42, N-4, A-153, I-44, E-138, G-61, A-130, R-70, N-28, P-35, S-149, K-103, Y-150, H-154, N-43, A-106, K-115, P-14, Y-5, K-137, E-141, E-87, E-73.

57 amino acids highly solvent exposed and conserved (>70%):

K-129, E-60, N-47, K-65, P-108, N-159, D-93, K-123, K-32, D-125, R-145, D-109, E-127, Q-36, E-131, L-152, E-6, E-96, D-156, P-63, H-76, E-8, K-134, E-45, T-10, V-12, K-20, S-155, H-126, P-50, N-78, K-119, V-2, L-24, E-42, N-4, A-153, I-44, E-138, G-61, A-130, R-70, N-28, P-35, S-149, K-103, Y-150, H-154, N-43, A-106, K-115, P-14, Y-5, K-137, E-141, E-87, E-73.

Table 1 shows a listing in descending order of solvent exposure of Bet v 1 amino acids. Column 1 lists the amino acid number starting from the amino-terminal, column 2 lists the amino acid in one letter abbreviation, column 3 lists the normalised solvent exposure index, column 4 lists the percent of known sequences having the concerned amino acid in this position.

TABLE 1
Bet v 1

| NO | AA | Solv_exp | Cons % |
|---|---|---|---|
| 129 | K | 1,000 | 90 |
| 60 | E | 0,986 | 97 |
| 47 | N | 0,979 | 100 |
| 65 | K | 0,978 | 100 |
| 108 | P | 0,929 | 100 |
| 159 | N | 0,869 | 100 |
| 93 | D | 0,866 | 100 |
| 123 | K | 0,855 | 100 |
| 32 | K | 0,855 | 100 |
| 125 | D | 0,821 | 74 |
| 145 | R | 0,801 | 90 |
| 109 | D | 0,778 | 82 |
| 77 | T | 0,775 | 56 |
| 127 | E | 0,760 | 100 |
| 36 | Q | 0,749 | 95 |
| 131 | E | 0,725 | 100 |
| 152 | L | 0,718 | 97 |
| 6 | E | 0,712 | 100 |
| 96 | E | 0,696 | 100 |
| 156 | D | 0,693 | 97 |
| 63 | P | 0,692 | 97 |
| 76 | H | 0,683 | 90 |
| 8 | E | 0,638 | 97 |
| 134 | K | 0,630 | 100 |
| 45 | E | 0,623 | 100 |
| 10 | T | 0,613 | 97 |
| 12 | V | 0,592 | 100 |
| 20 | K | 0,584 | 100 |
| 62 | L | 0,575 | 5 |
| 155 | S | 0,568 | 97 |
| 126 | H | 0,551 | 95 |
| 50 | P | 0,541 | 100 |
| 78 | N | 0,538 | 100 |
| 119 | K | 0,529 | 100 |
| 2 | V | 0,528 | 100 |
| 24 | L | 0,528 | 100 |
| 42 | E | 0,519 | 100 |
| 4 | N | 0,517 | 95 |
| 153 | A | 0,513 | 100 |
| 44 | I | 0,508 | 97 |
| 138 | E | 0,496 | 100 |
| 61 | G | 0,488 | 100 |
| 130 | A | 0,479 | 97 |
| 70 | R | 0,474 | 100 |
| 28 | N | 0,469 | 90 |
| 35 | P | 0,467 | 100 |
| 149 | S | 0,455 | 92 |
| 103 | K | 0,447 | 100 |
| 150 | Y | 0,438 | 100 |
| 154 | H | 0,436 | 100 |
| 43 | N | 0,412 | 100 |
| 106 | A | 0,411 | 95 |
| 115 | K | 0,411 | 100 |
| 14 | P | 0,410 | 97 |
| 5 | Y | 0,410 | 100 |
| 137 | K | 0,396 | 100 |
| 141 | E | 0,387 | 95 |
| 87 | E | 0,385 | 100 |
| 73 | E | 0,384 | 100 |
| 16 | A | 0,367 | 100 |
| 79 | F | 0,362 | 100 |
| 3 | F | 0,355 | 100 |
| 158 | Y | 0,346 | 100 |
| 105 | V | 0,336 | 100 |
| 101 | E | 0,326 | 100 |
| 64 | F | 0,325 | 100 |
| 86 | I | 0,322 | 100 |
| 39 | S | 0,314 | 100 |
| 124 | G | 0,310 | 100 |
| 72 | D | 0,308 | 97 |
| 142 | T | 0,293 | 67 |
| 66 | Y | 0,289 | 100 |
| 55 | K | 0,288 | 100 |
| 7 | T | 0,279 | 67 |
| 40 | S | 0,274 | 95 |
| 25 | D | 0,271 | 87 |
| 135 | A | 0,267 | 92 |
| 68 | K | 0,262 | 100 |
| 97 | K | 0,247 | 100 |
| 46 | G | 0,235 | 100 |
| 27 | D | 0,232 | 97 |
| 1 | G | 0,227 | 100 |
| 113 | I | 0,225 | 77 |
| 51 | G | 0,220 | 100 |
| 92 | G | 0,218 | 100 |
| 80 | K | 0,212 | 100 |
| 110 | G | 0,211 | 100 |
| 107 | T | 0,203 | 85 |
| 94 | T | 0,202 | 92 |
| 41 | V | 0,201 | 97 |
| 48 | G | 0,198 | 100 |
| 91 | I | 0,192 | 18 |
| 31 | P | 0,188 | 100 |
| 75 | D | 0,188 | 97 |
| 33 | V | 0,183 | 100 |
| 49 | G | 0,176 | 100 |
| 17 | R | 0,172 | 100 |
| 99 | S | 0,158 | 64 |
| 89 | G | 0,154 | 100 |
| 53 | I | 0,154 | 100 |
| 121 | H | 0,153 | 100 |
| 9 | T | 0,150 | 72 |
| 74 | V | 0,148 | 97 |
| 132 | Q | 0,146 | 72 |
| 57 | S | 0,137 | 49 |
| 148 | E | 0,135 | 100 |
| 82 | N | 0,133 | 41 |
| 128 | V | 0,125 | 64 |
| 117 | S | 0,124 | 87 |
| 90 | P | 0,117 | 67 |
| 116 | I | 0,112 | 100 |
| 122 | T | 0,107 | 100 |
| 139 | M | 0,104 | 62 |
| 95 | L | 0,104 | 97 |
| 54 | K | 0,096 | 100 |
| 146 | A | 0,095 | 100 |
| 59 | P | 0,088 | 97 |
| 157 | A | 0,088 | 100 |
| 133 | V | 0,077 | 44 |
| 88 | G | 0,068 | 100 |
| 140 | G | 0,053 | 85 |
| 37 | A | 0,042 | 95 |
| 81 | Y | 0,041 | 100 |
| 23 | I | 0,036 | 95 |
| 104 | I | 0,036 | 92 |
| 15 | A | 0,036 | 97 |
| 58 | F | 0,029 | 100 |
| 29 | L | 0,028 | 100 |
| 19 | F | 0,027 | 100 |
| 100 | N | 0,022 | 97 |
| 22 | F | 0,021 | 97 |
| 71 | V | 0,014 | 100 |
| 111 | G | 0,014 | 100 |
| 13 | I | 0,014 | 100 |
| 18 | L | 0,014 | 97 |
| 114 | L | 0,014 | 100 |
| 11 | S | 0,007 | 100 |
| 151 | L | 0,007 | 97 |
| 144 | L | 0,007 | 90 |
| 52 | T | 0,007 | 100 |
| 84 | S | 0,007 | 97 |
| 118 | N | 0,007 | 97 |
| 102 | I | 0,007 | 100 |
| 21 | A | 0,000 | 97 |
| 26 | G | 0,000 | 97 |
| 30 | F | 0,000 | 44 |
| 34 | A | 0,000 | 100 |
| 38 | I | 0,000 | 87 |
| 56 | I | 0,000 | 100 |
| 67 | V | 0,000 | 97 |
| 69 | D | 0,000 | 62 |
| 83 | Y | 0,000 | 95 |
| 85 | V | 0,000 | 72 |
| 98 | I | 0,000 | 95 |
| 112 | S | 0,000 | 77 |
| 120 | Y | 0,000 | 95 |
| 136 | S | 0,000 | 67 |
| 143 | L | 0,000 | 100 |
| 147 | V | 0,000 | 100 |

### Example 4

This Example describes preparation and characterisation of recombinant mutant Bet v 1 allergens with more than four mutations and diminished IgE-binding affinity according to prior art PCT/DK 01/00764. Mutants according to the present invention are prepared and assayed accordingly.

Selection of Amino Acid Residues for Site-Directed Mutagenesis of Bet v 1

Amino acid residues were selected as described in Example 1.

In Vitro Mutagenesis

In vitro mutagenesis was performed by PCR using recombinant pMAL-c with Bet v 1 inserted as template. Preparation of recombinant mutant allergens comprising five to nine primary mutations included two PCR steps; step I and II. First, each single mutation (or several mutations if located closely together in the DNA sequence) was introduced into sequential DNA sequences of Bet v 1.2801 or Bet v 1.2801 derivatives using sense and anti-sense mutation-specific oligonucleotide primers accommodating each mutation(s) along with sense and anti-sense oligonucleotide primers accommodating either upstream or downstream neighbour mutations or the N-terminus/C-terminus of Bet v 1, respectively as schematically illustrated in FIG. 15 (I). Secondly, PCR products from PCR reaction I were purified, mixed and used as templates for an additional PCR reaction (II) with oligonucleotide primers accommodating the N-terminus and C-terminus of Bet v 1 as schematically illustrated in FIG. 15 (II). The PCR products were purified by agarose gel electrophoresis and PCR gel purification (Life Techhnologies) followed by ethanol precipitation, cut with restriction enzymes (SacI/EcoRI) or (SacI/XbaI), and ligated directionally into pMAL-c restricted with the same enzymes.

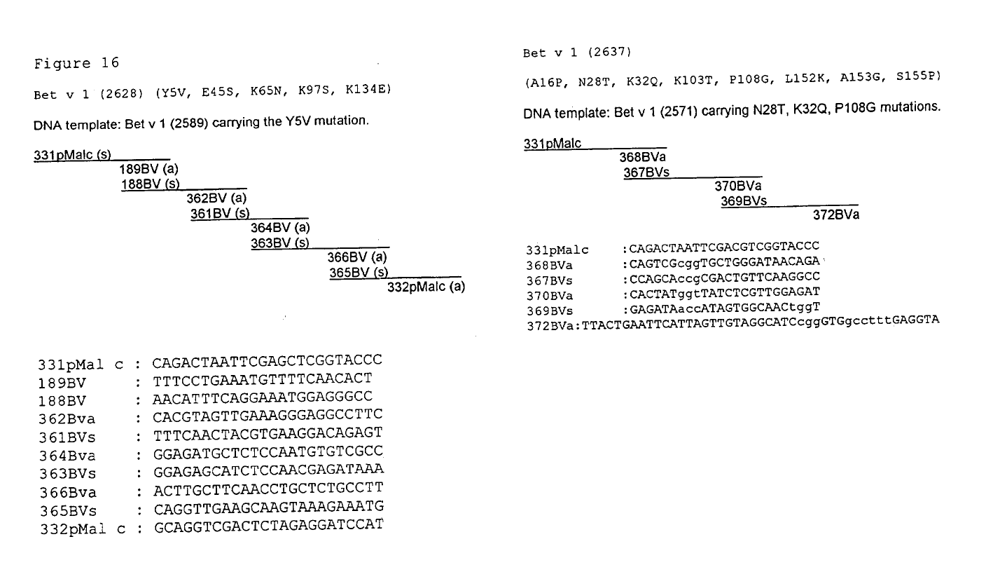

FIG. 16 shows synthesised oligonucleotide primers and schematically illustrations for the construction of Bet v 1 mutants with more than four primary mutations. The mutated amino acids were preferably selected from the group consisting of amino acids that are characterised by being highly solvent exposed and conserved as described in Example 3. The Bet v 1 mutants are as follows:

Mutant Bet v 1 (2628): Tyr5Val, Glu45Ser, Lys65Asn, Lys97Ser, Lys134Glu.

Mutant Bet v 1 (2637): Ala16Pro, Asn28Thr, Lys32Gln, Lys103Thr, Pro108Gly, Leu152Lys, Ala153Gly, Ser55Pro.

Mutant Bet v 1 (2733): Tyr5Val, Lys134Glu, Asn28Thr, Lys32Gln, Glu45Ser, Lys65Asn, Asn78Lys, Lys103Val, Lys97Ser, Pro108Gly, Arg145Glu, Asp156His, +160Asn.

Mutant Bet v 1 (2744): Tyr5Val, Lys134Glu, Glu42Ser, Glu45Ser, Asn78Lys, Lys103Val, Lys123Ile, Asp156His, +160Asn.

Mutant Bet v 1 (2753): Asn28Thr, Lys32Gln, Lys65Asn, Glu96Leu, Lys97Ser, Pro108Gly, Asp109Asn, Asp125Tyr, Glu127Ser, Arg145Glu.

Nucleotide Sequencing and Expression and Purification of Recombinant Bet v 1 and Mutants

Sequencing and expression of recombinant protein was performed as described in Example 1.

Bet v 1 (2628) and Bet v 1 (2637) Mutants

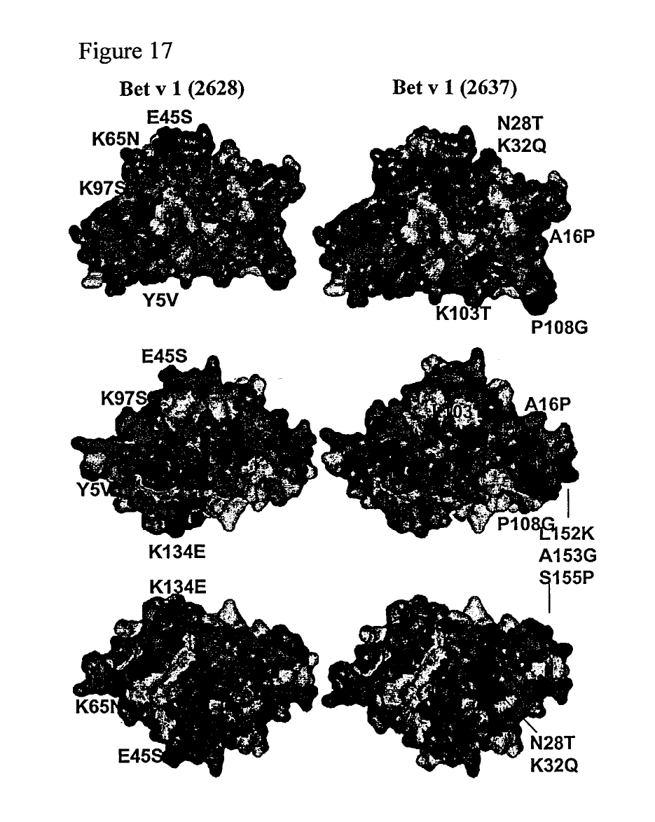

FIG. 17 shows introduced point mutations at the molecular surface of Bet v 1 (2628) and Bet v 1 (2637).

Crystallisation and Structural Determination of Recombinant Bet v 1(2628) Mutant Protein.

Structural determination was performed as described in Example 1.

Structure of Bet v 1 (2628) Mutant

The structural effect of the mutations was addressed by growing three-dimensional Bet v 1 (2628) protein crystals diffracting to 2.0 Å resolution when analysed by X-rays generated from a rotating anode. The substitutions Tyr5Val, Glu45Ser, Lys65Asn, Lys97Ser, Lys134Glu were verified by the Bet v 1 (2628) structure electron density map which also showed that the overall α-carbon backbone tertiary structure is preserved.

Structural Analysis of Bet v 1 (2637) Mutant

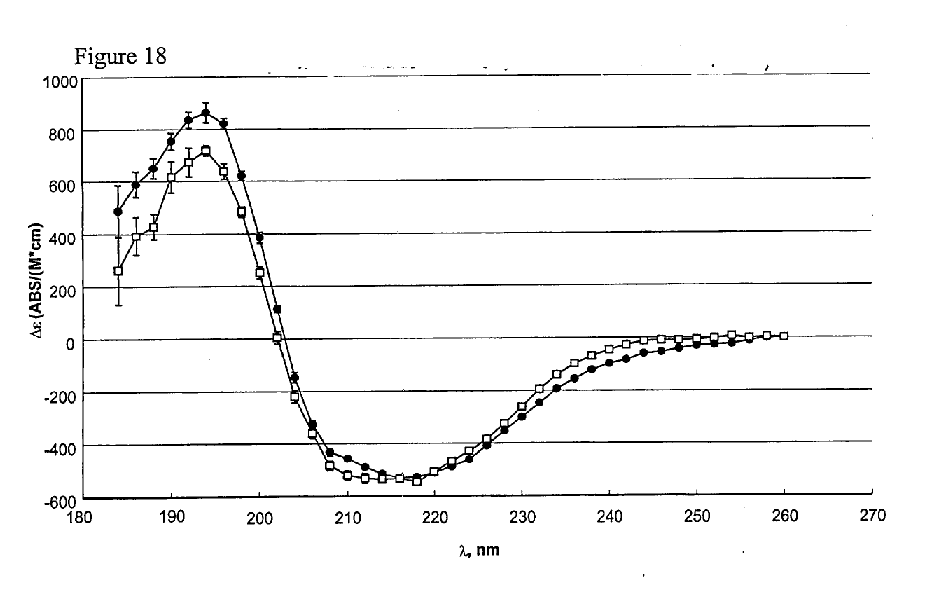

The structural integrity of the purified Bet v 1 (2637) mutant was analysed by circular dichroism (CD) spectroscopy. FIG. 18 shows the CD spectra of recombinant Bet v 1.2801 (wildtype) and Bet v 1 (2637) mutant, recorded at close to equal concentrations. The overlap in peak amplitudes and positions in the CD spectra from the two recombinant proteins shows that the two preparations contain equal amounts of secondary structures strongly suggesting that the α-carbon backbone tertiary structure is not affected by the introduced amino acid substitutions.

IgE-Binding Properties of Bet v 1 (2628) and Bet v 1 (2637) Mutants.

The IgE-binding properties of Bet v 1 (2628) and Bet v 1 (2637) as well as a 1:1 mix of Bet v 1 (2628) and Bet v 1 (2637) was compared with recombinant wild type Bet v 1.2801 in a fluid-phase IgE-inhibition assay using a pool of serum IgE derived from birch allergic patients.

As described in Example 1, recombinant Bet v 1.2801 was biotinylated at a molar ratio of 1:5 (Bet v 1 no. 2801:biotin). The inhibition assay was performed as follows: a serum sample (25 μl) was incubated with solid phase anti IgE, washed, re-suspended and further incubated with a mixture of biotinylated Bet v 1.2801 and a given mutant or 1:1 mix of the two mutants. The amount of biotinylated Bet v 1.2801 bound to the solid phase was estimated from the measured RLU after incubation with acridinium ester labelled streptavidin. The degree of inhibition was calculated as the ratio between the RLU's obtained using buffer and mutant as inhibitor.

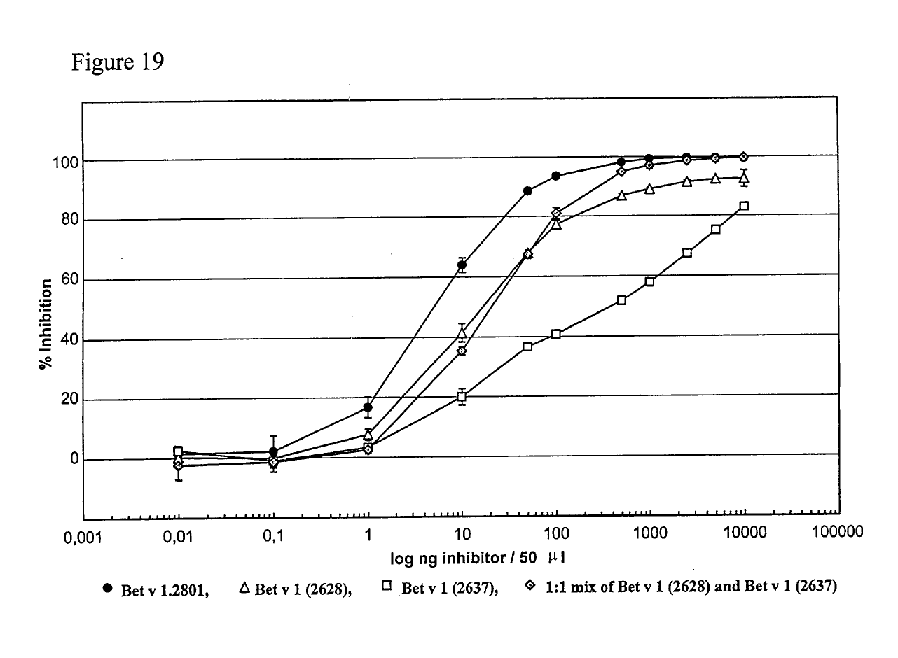

FIG. 19 shows the inhibition of the binding of biotinylated recombinant Bet v 1.2801 to serum IgE from a pool of allergic patients by non-biotinylated Bet v 1.2801 and by Bet v 1 (2628), Bet v 1 (2637) and a 1:1 mix of Bet v 1 (2628) and Bet v 1 (2637).

There is a clear difference in the amount of respective recombinant proteins necessary to reach 50% inhibition of the binding to serum IgE present in the serum pool. Recombinant Bet v 1.2801 reaches 50% inhibition at about 5 ng whereas the corresponding concentration for Bet v 1 (2628) mutant is about 15-20 ng. This show that the point mutation introduced in the Bet v 1 (2628) mutant lowers the affinity for specific serum IgE by a factor of about 3-4.

The maximum level of inhibition reached by the Bet v 1 (2628) mutant protein is clearly lower compared to recombinant Bet v 1.2801. This may indicate that some of the specific IgE present in the serum pool are unable to recognise the Bet v 1 (2628) mutant protein due to the introduced point mutations.

Bet v 1 (2637) reaches 50% inhibition at about 400-500 ng showing that the point mutation introduced in the Bet v 1 (2637) mutant lowers the affinity for specific serum IgE by 80 to 100-fold compared to Bet v 1.2801. The large difference in IgE-binding is further supported by a clear difference in inclination of the inhibition curve obtained with Bet v 1(2637) mutant protein compared to the inhibition curve for Bet v 1.2801. The different inclination provide evidence that the reduction in IgE-binding is due to a distinctly different epitope pattern of the mutant compared to Bet v 1.2801.

In addition to the inhibition assays with single modified allergens a 1:1 mix of Bet 1 (2628) and Bet v 1 (2637) having same molar concentration of Bet v 1 as each of the samples with Bet 1 (2628) or Bet v 1 (2637), respectively was tested and showed full (100%) capacity to inhibit IgE-binding to rBet v 1.2801. The capacity to fully inhibit IgE-binding is a clear indication that all reactive epitopes present on Bet v 1.2801 were present in the 1:1 allergen mix. Further support comes from the comparable inclination of the two inhibition curves for Bet v 1.2801 and the allergen mix. Reduced IgE-reactivity of the mixed allergen sample is demonstrated by the need of a four-fold higher concentration of the allergen mix, when compared to Bet v 1.2801, for obtaining 50% inhibition of IgE-binding.

T Cell Proliferation Assay using Mutated Recombinant Bet v 1 Allergens.

The analysis was carried out as described in ref. 15. Bet v 1 (2628) and Bet v 1(2637) mutant protein were both able to induce proliferation in T cell lines from birch pollen allergic patients with stimulation indices similar to recombinant and naturally occurring. This suggests that both of Bet v 1 (2628) and Bet v 1 (2637) mutant protein can each initiate the cellular immune response necessary for antibody production.

Histamine Release Assays with Human Basophil.

Histamine release from basophil leucocytes was performed as follows. Heparinized blood (20 ml) was drawn from each birch pollen patient, stored at room temperature, and used within 24 hours. Twenty-five microlitres of heparinized whole blood was applied to glass fibre coated microtitre wells (Reference Laboratory, Copenhagen, Denmark) and incubated with 25 microlitres of allergen or anti-IgE for 1 hour at 37° C. Thereafter the plates were rinsed and interfering substances were removed. Finally, histamine bound to the microfibres was measured spectrophotofluometrically.

Histamine Release Properties of Bet v 1 (2628) and Bet v 1 (2637) Mutant Protein.

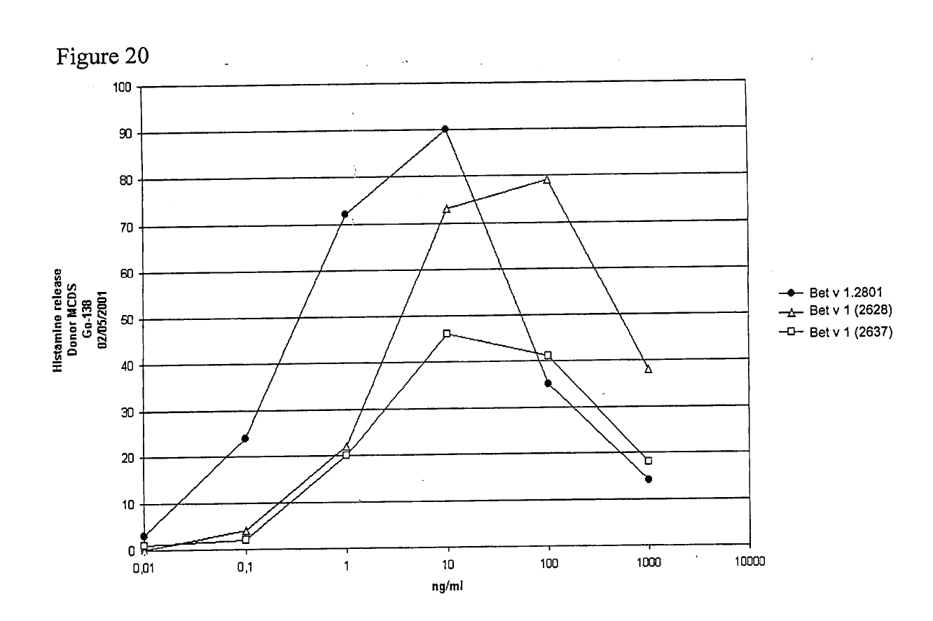

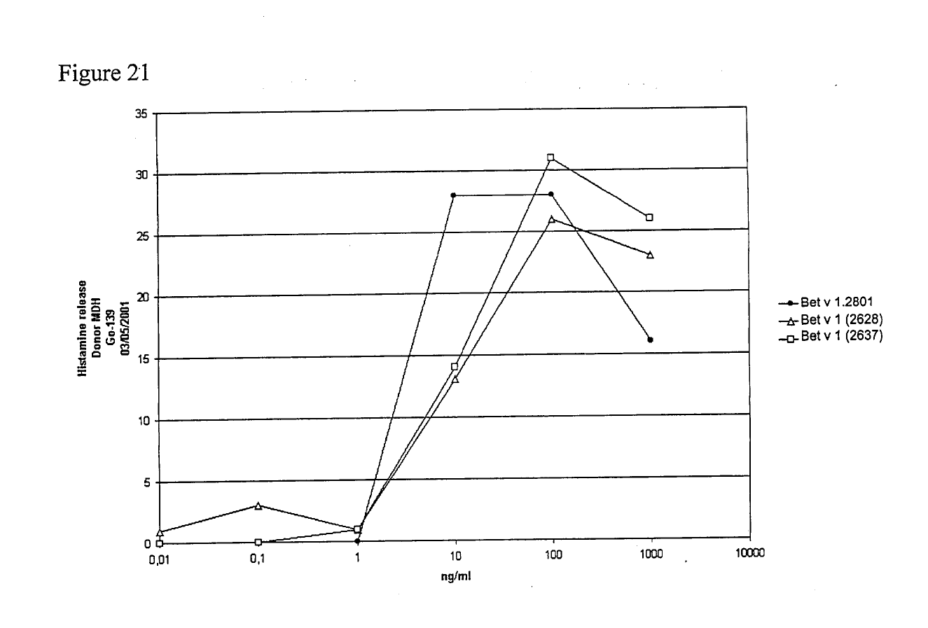

Histamine release data is shown in FIG. 20 and FIG. 21. The potency of Bet v 1 (2628) and Bet v 1 (2637) mutant protein to induce histamine release in human basophil from two birch pollen allergic patients has been tested. In both cases the release curve of the mutated allergens to induce histamine release is clearly shifted to the right compared to the release curve of Bet v 1.2801. The shift indicate that the potency of Bet v 1 (2628) and Bet v 1 (2637) is reduced 3 to 10-fold.

Mutant Bet v 1 (2744) and Mutant Bet v 1 (2753)

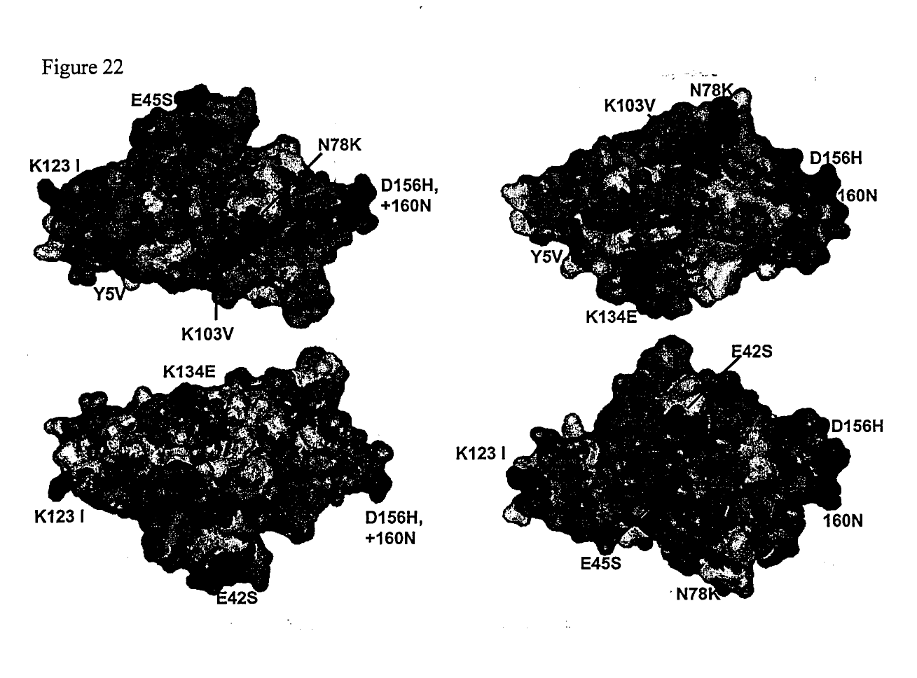

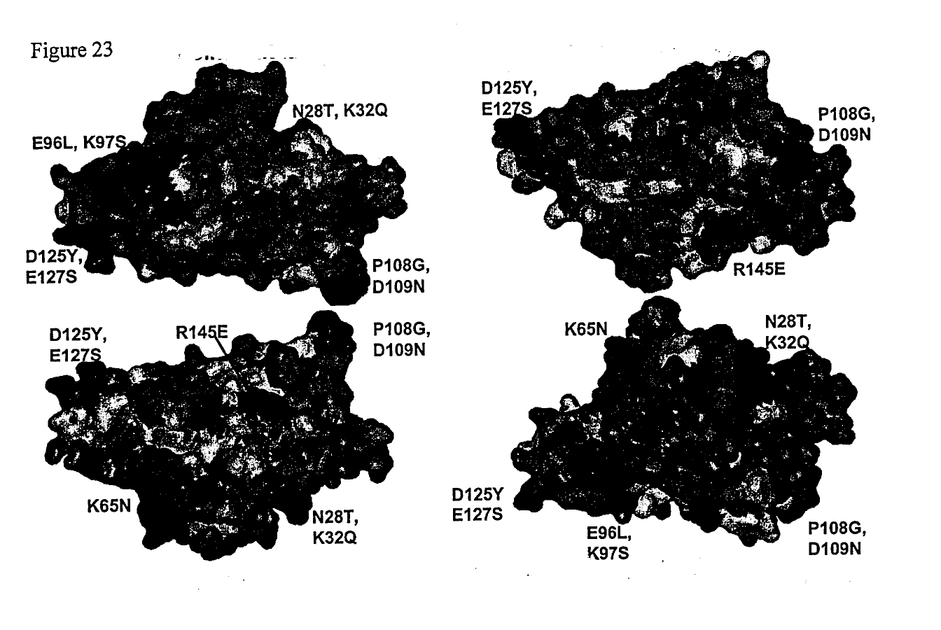

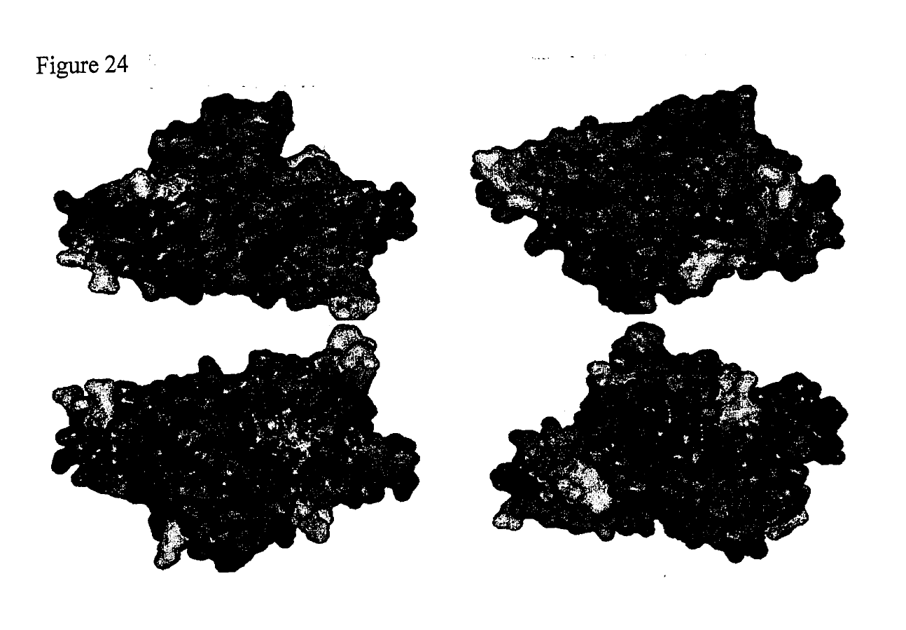

Bet v 1 (2744) and Bet v 1 (2753) was likewise constructed for use as a mixed allergen vaccine. In these mutated allergens point mutations were distributed in an all surface arranged fashion as shown in FIG. 22 and FIG. 23 and was again designed to affect different surface areas in the two molecules, respectively, as shown in FIG. 24. However these modified allergens might individually be used as single allergen vaccines as well.

Structural Analysis of Bet v 1 (2744) Mutant Protein

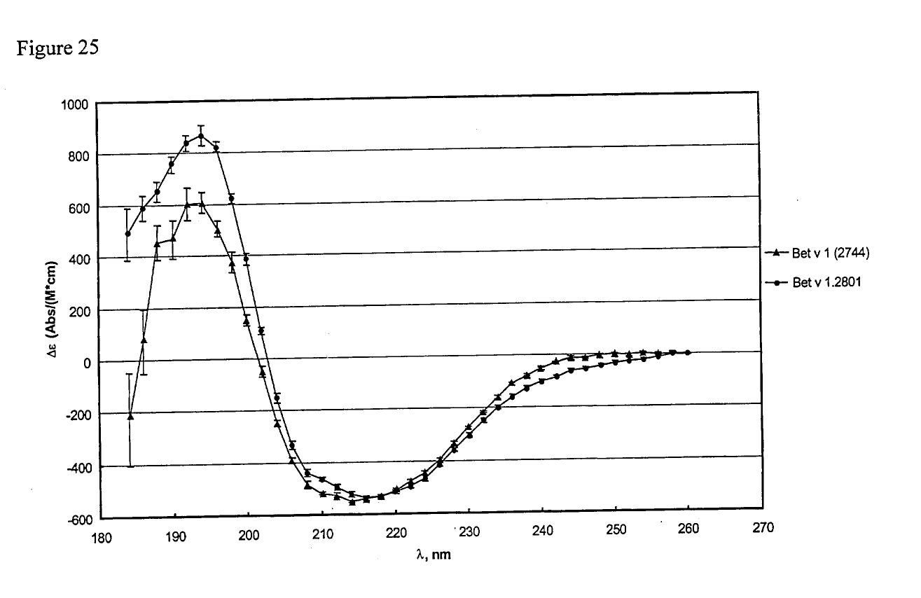

The structural integrity of the purified Bet v 1 (2744) mutant was analysed by circular dichroism (CD) spectroscopy. FIG. 25 shows the CD spectra of recombinant Bet v 1.2801 (wildtype) and Bet v 1 (2744) mutant, recorded at close to equal concentrations. The overlap in peak amplitudes and positions in the CD spectra from the two recombinant proteins shows that the two preparations contain equal amounts of secondary structures strongly suggesting that the α-carbon backbone tertiary structure is not affected by the introduced amino acid substitutions.

Histamine Release Properties of Bet v 1 (2744)

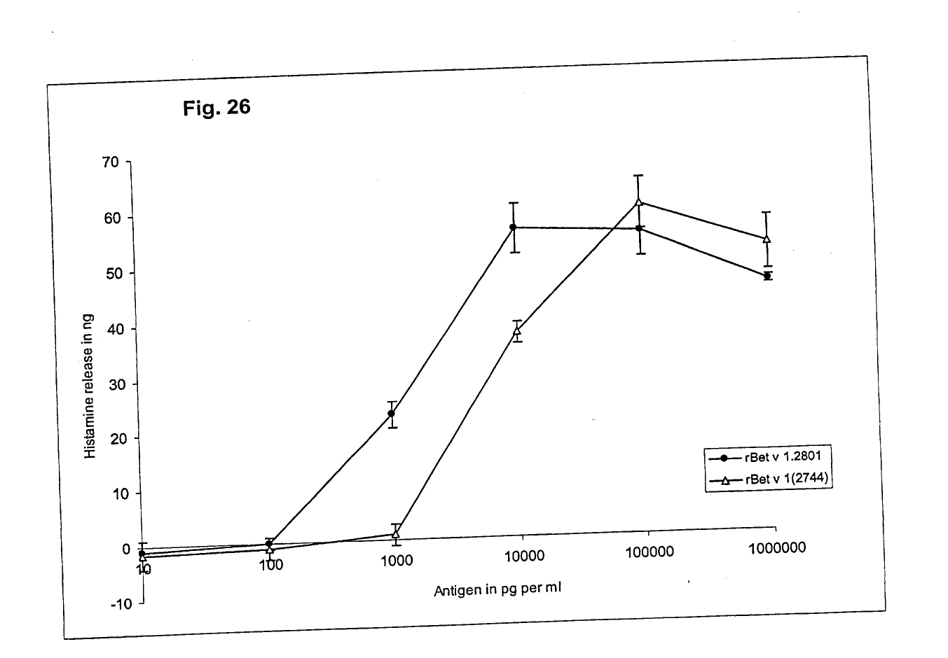

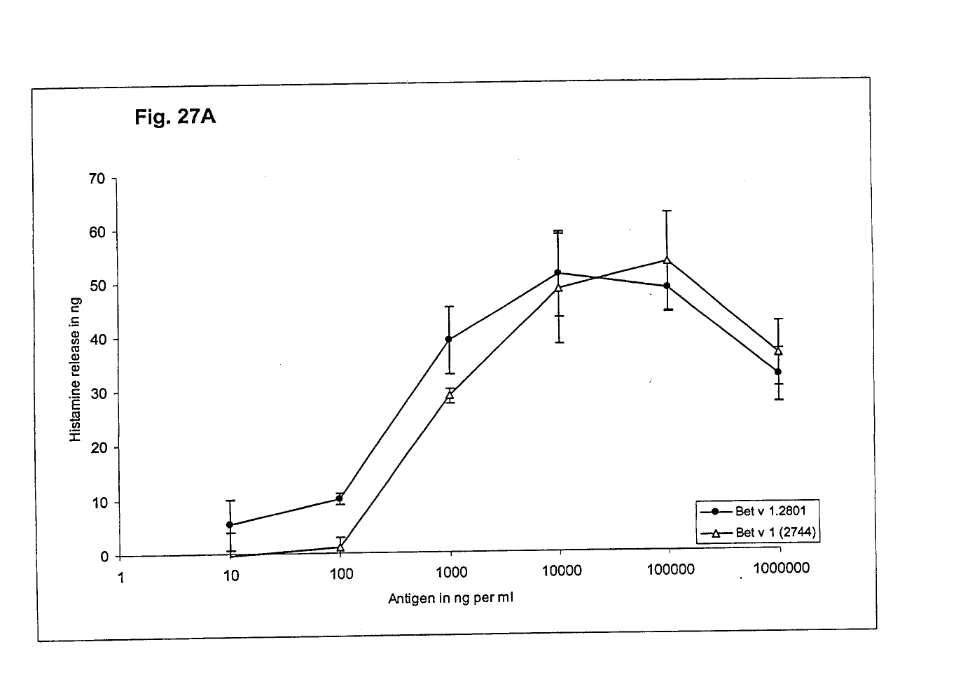

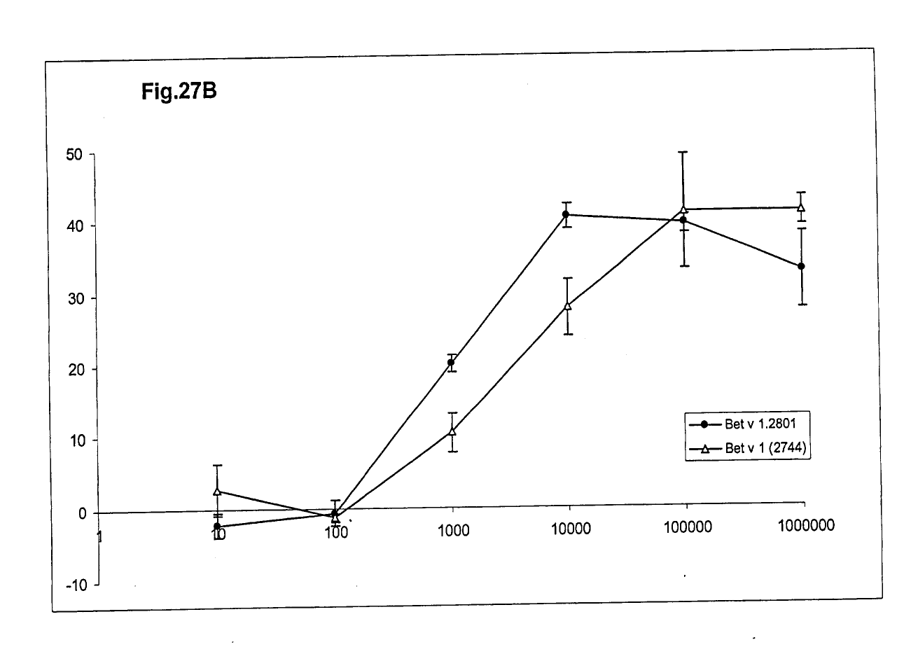

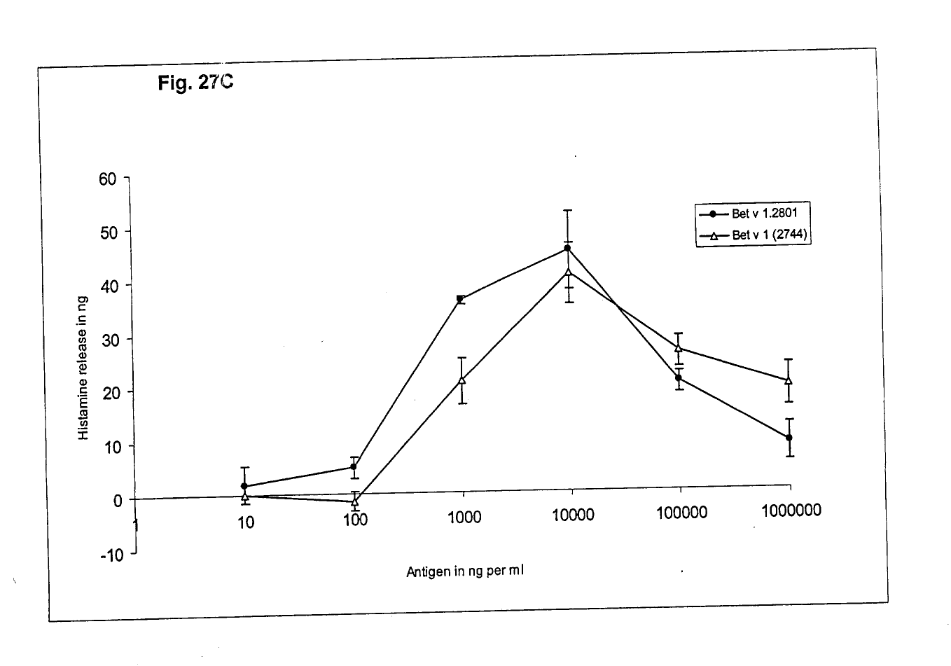

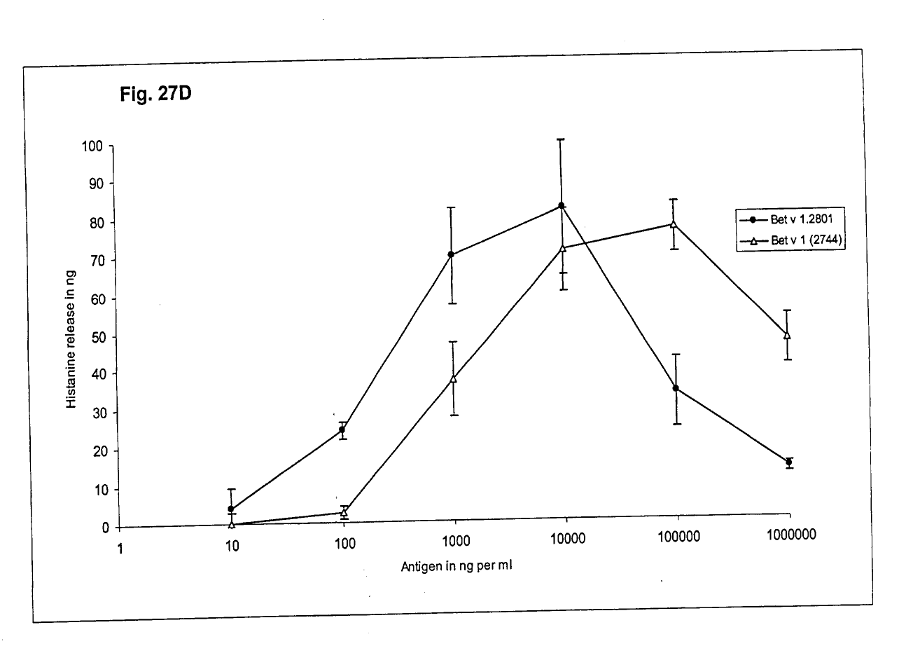

Histamine release data from five experiments with basophil leucocytes from five different birch pollen allergic patients is shown in FIG. 26 and FIGS. 27A-D. The potency of Bet v 1 (2744) mutant protein to induce histamine release in human basophil has been tested. The release curves of the mutated allergens are clearly shifted to the right compared to the release curve of Bet v 1.2801 indicating that the potency of Bet v 1 (2744) to release histamine is reduced 3 to 5-fold.

Mutant Bet v 1 (2733)

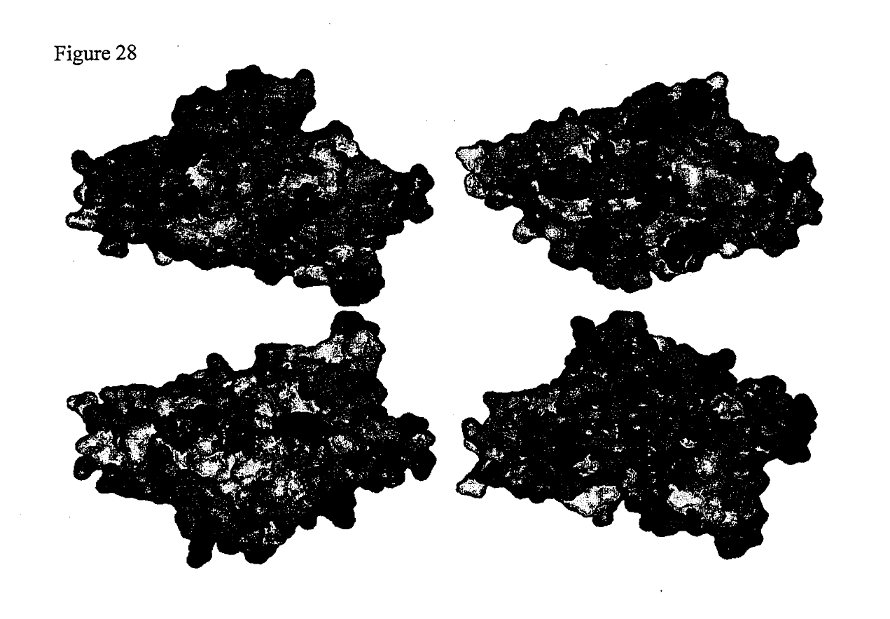

A Mutant Bet v 1 (2733) has been constructed and recombinantly expressed. The distribution of point mutations in Bet v 1 (2733) leave several surface areas constituting >400Å² unaltered. FIG. 28 show introduced point mutations at the molecular surface of Bet v 1 (2733).

### Example 5

This Example describes characterisation of recombinant mutant Bet v 1 allergens with more than four mutations and diminished IgE-binding affinity according to prior art PCT/DK 01/00764. Mutants according to the present invention are prepared and assayed accordingly.

T-Cell Reactivity of Recombinant and Mutant Bet v 1:

Purpose:

To investigate an in vitro T-cell response to the mutated allergens in terms of proliferation and cytokine production.

Methods:

PBL (Peripheral blood lymphocytes) from allergic patients were used in the following investigation.

Eight bet v 1 specific T-cell lines were established from the PBL with naturally purified bet v 1 in order to sustain the variety of bet v 1 isoforms the T-cells are presented to, as described in a previously published protocol (26).

Ten PBL and eight T-cell lines were stimulated with birch extract (Bet v), naturally purified bet v 1 (nBet v 1), recombinant Bet v 1 (rBet v 1 or wt; 27) and four different mutated forms of rBet v 1 (described elsewhere): 2595, 2628, 2637, 2744, 2773. The 2637 mutant was later found to be partly unfolded and will not be discussed.

In brief: In a round-bottomed 96 well plate PBL were added in 2×10⁵ per well. The different birch samples were added in three different concentrations in quadroplicates and allowed to grow for 6 days. At day 6 cell half of volume (100 μl) from each well with the highest concentration of birch were harvested for cytokine production. Radioactive labelled thymidine was added to the wells. Next day (day 7) the cells were harvested on a filter. Scintilation fluid was added to the filter and the radioactivity was measured in a scintillation counter.

Likewise in a 96 well round-bottomed 96 well plate T-cells were added in 3×10⁴ T-cells per well and stimulated with irradiated autologous PBL (1×10⁵ cells/well) and 3 different concentrations of the different birch samples. After 1 day cells from each well with the highest concentration birch were harvested for cytokine production. Radioactive labelled thymidine were added to the wells. At day 2 the cells were harvested onto a filter and counted as described for PBL.

Supernatant from the quadroplicates were pooled and cytokines were measured using a CBA (cytokine bead array) kit from Becton Dickinson.

Results:

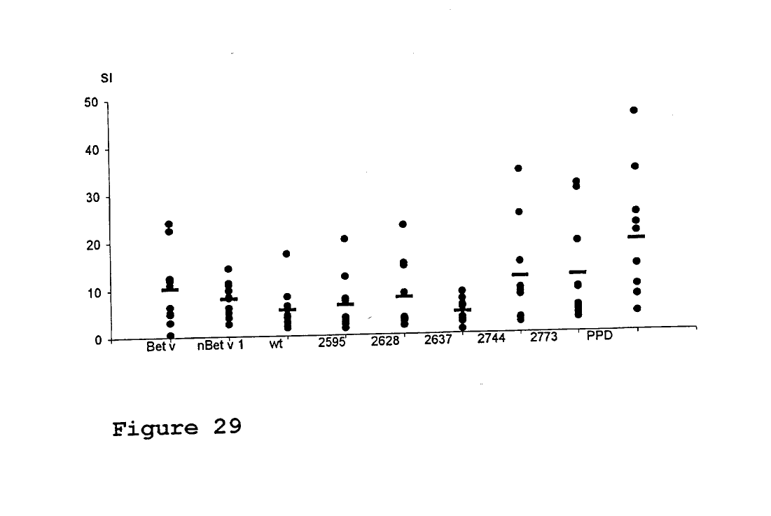

Ten PBL cultures showed specific stimulation to birch. In general proliferation of the PBL to the different birch samples were similar, although variations could be seen. In 3 PBL, nBet v 1 stimulated proliferation better than rBet v 1 and the mutants. The mutant birch samples stimulated PBL almost identical to rBet v 1 (FIG. 29). FIG. 29 shows the Stimulation Index for the above-mentioned Bet v 1 preparations. The Stimulation Index (SI) is calculated as proliferation (cpm: count per minute) of the stimulated sample (highest concentration) divided with the proliferation (cpm) of the medium control. PPD designates purified protein derivative from mucobacterium tuberculosis, which serves as a positive control.

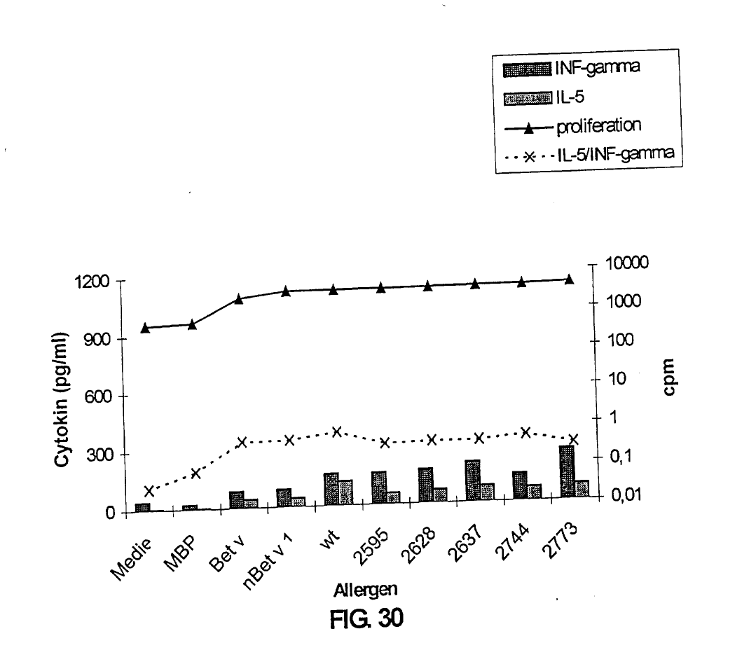

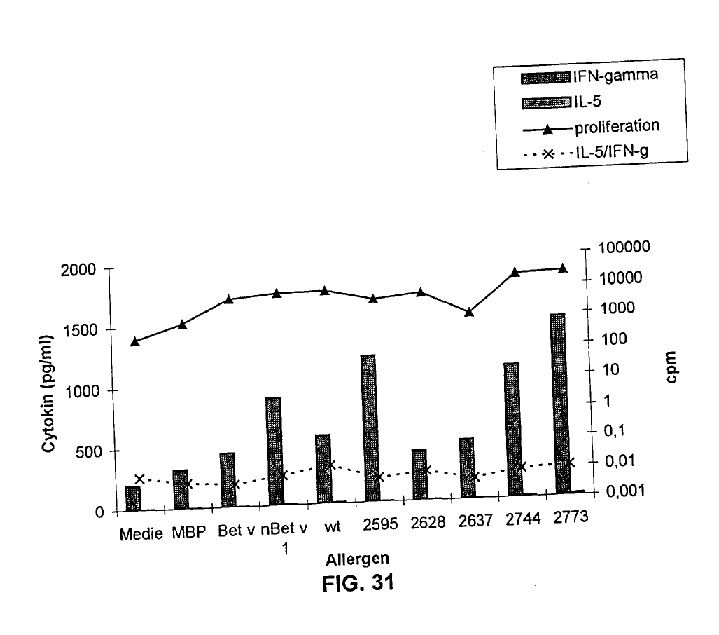

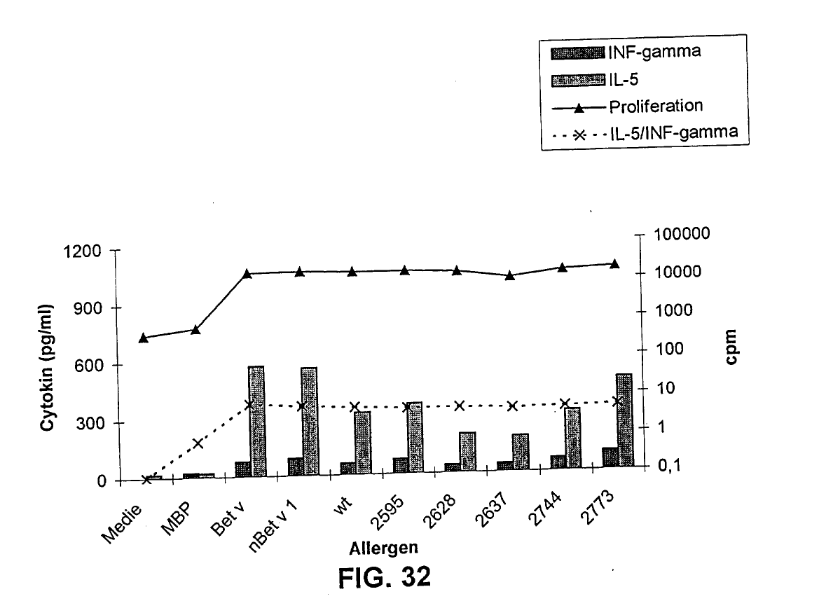

Cytokine production was dominated by IFN-gamma and increased proportionally with PBL proliferation. No signs of a Th1/Th2 shift were apparent (FIGS. 30-32). FIG. 30 shows a patient with a Th0 profile, FIG. 31 a Th1 profile and FIG. 32 a Th2 profile. Cytokine production is measured in pg/ml indicated as the bars and the ratio between IL-5/IFN-gamma is the lower dashed line (Y-axis to the right). Proliferation is measured in cpm seen on the Y-axis to the right as a solid line measured in cpm. Medium and MBP (maltose bindig protein) are included as background controls.

Eight T-cell lines established on nBet v 1 and all, except one, proliferated equally well to all birch samples. Four T-cell lines were secreting Th0 like cytokines based on the IL-5 and IFN-gamma ratio (Th2>5, 5>Th0>0.2, 0.2>Th1). Three T-cell lines were secreting Th1 cytokines and one T-cell line was secreting Th2 cytokines. The IL-5/IFN-gamma ratio was not affected by the different birch samples.

Conclusion:

All PBL cultures and ⅞ T-cell lines that showed specific stimulation to nBet v 1 did also respond to rBet v 1 and the mutants. These data suggests that for T-cell stimulation a single isoform of Bet v 1 or these 4 mutants can substitute for the mixture of individual isoforms found in the natural allergen preparations. Thus, vaccines based on recombinant allergens or these 4 mutants will address the existing Bet v 1 specific T-cell population.

### Example 6

This Example describes characterisation of recombinant mutant Bet v 1 allergens with more than four mutations and diminished IgE-binding affinity according to prior art PCT/DK 01/00764. Mutants according to the present invention are be prepared and assayed accordingly.

Induction of Bet v 1 Specific IgG Antibodies and Blocking Antibodies Following Immunization with Recombinant and Mutant Bet v 1 Proteins:

In this section the term “blocking antibodies” is defined as antibodies, different from human IgE antibodies, that are able to bind to an antigen and prevent the binding of human IgE antibodies to that antigen.

The ability of recombinant Bet v1 2227 wild type protein (rBet v 1) and Bet v 1 2595, 2628, 2744 and 2773 mutant proteins to induce Bet v 1 specific IgG antibodies and blocking antibodies was tested in immunization experiments in mice.

BALB/cA mice (8 in each group) were immunized by intraperitoneal injections with recombinant Bet v1 2227 wild type protein or the four mutant proteins. The mice were immunized four times with a dose interval of 14 days. The different proteins were conjugated to 1,25 mg/ml Alhydrogel, (Aluminium Hydroxide gel, 1,3% pH 8.0-8.4, Superfos Biosector). The mice were immunized with either 1 ug protein/dose or 10 ug protein/dose. Blood samples were drawn by orbital bleed at day 0,14,35, 21, 49 and 63.

Specific IgG antibody levels was analyzed by direct ELISA using rBet v 1 coated microtiterplates and biotinylated rabbit anti mouse IgG antibodies (Jackson) as detection antibody. Immunization with recombinant Bet v1 2227 wild type protein or the four mutant proteins induced a strong r Bet v 1 specific IgG response. This finding demonstrates that the four mutated proteins are able to induce antibodies that are highly cross reactive to the Bet v 1 2227 wild type protein.

To assess the induction of blocking antibodies, serum samples from birch pollen allergic patients were incubated with paramagnetic beads coated with a monoclonal mouse anti-human IgE antibody. After incubation, the beads were washed and resuspended in buffer or diluted samples (1:100) of mouse serum from un-immunized mice (control) or mice immunized as described above. Biotinylated r Bet v 1 was then added to this mixture of beads and mouse serum antibodies. After incubation, the beads were washed and bound biotinylated rBet v 1 was detected using acridinium labeled streptavidine. Incubation of beads with serum from un-immunized mice did not change the binding of r Bet v 1 to the beads. In contrast, incubation of the beads with serum from mice immunized with the recombinant Bet v1 2227 wild type protein or the four mutant proteins significantly reduced binding of r Bet v 1 to the beads demonstrating the presence of Bet v 1 specific blocking antibodies in the serum samples. Thus, at day 63 one or more serum samples from all high dose (10 ug/dose) immunization groups were able to reduce binding of r Bet v1 to the beads with more than 80%. These findings demonstrate that the four mutated proteins are able to induce antibodies that can act as Bet v 1 specific blocking antibodies.

### Example 7

This example describes the structural characterization and IgE-binding properties of a mutant according to the invention having 12 point mutation. The mutations introduced in mutant 3007 are described in example 2.

Structural Analysis of Bet v 1 (3007) Mutant Protein

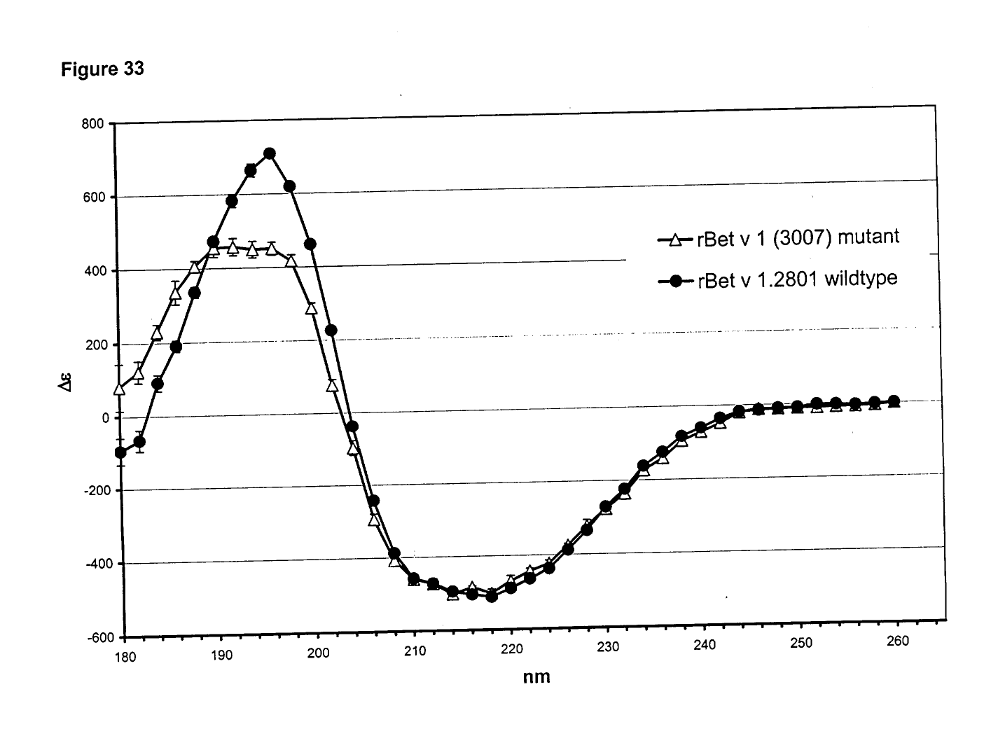

The structural integrity of the purified Bet v 1 (3007) mutant was analysed by circular dichroism spectroscopy as described in example 1. FIG. 33 shows the CD spectra of recombinant Bet v 1.2801 (wildtype) and Bet v 1 (3007) mutant, recorded at equal concentrations as previously described in example 1. The overlap in amplitude-positions in the CD spectra from the two recombinant proteins indicates that the two preparations contain roughly equal amounts of secondary structures, strongly suggesting that the α-carbon backbone tertiary structure is no t or affected by the introduced amino acid substitutions.

IgE-Binding Analysis of Bet v 1 (3007) Mutant Protein

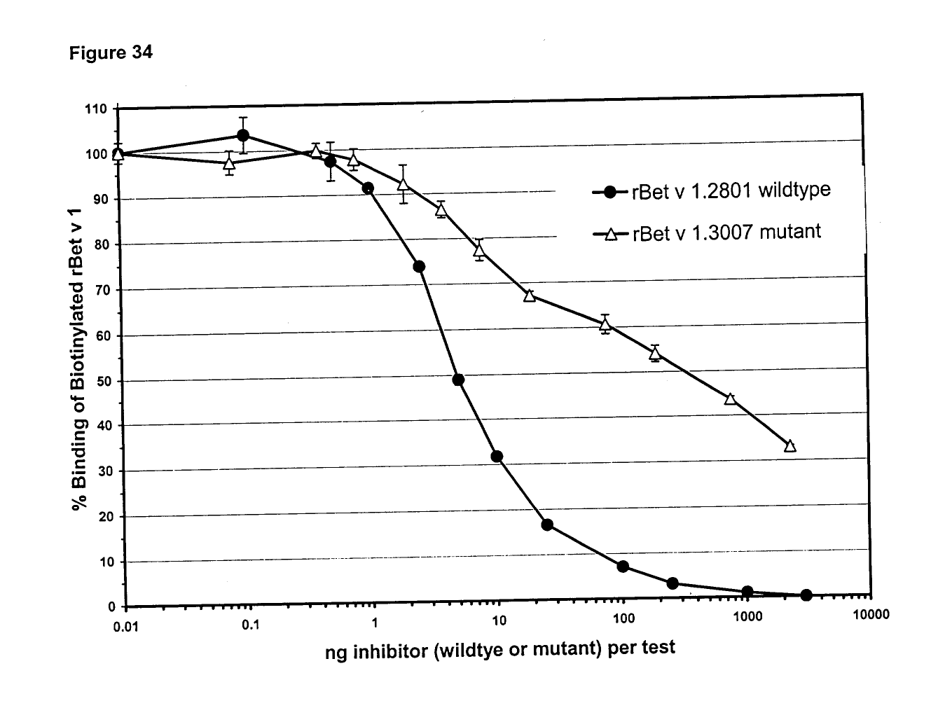

FIG. 34 shows the inhibition of the binding of biotinylated recombinant Bet v 1.2801 to serum IgE from a pool of allergic patients by non-biotinylated Bet v 1.2801 (wildtype) and the Bet v 1 (3007) mutant according to methods described in example 4. There is a clear difference in the amount of the respective recombinant proteins necessary to reach 50% inhibition of the binding to serum IgE present in the serum pool. Recombinant Bet v 1.2801 reaches 50% inhibition at about 5 ng whereas the corresponding concentration for Bet v 1 (3007) mutant is about 200 ng. The level of inhibition reached by the Bet v 1 (3007) mutant protein is clearly lower compared to recombinant Bet v 1.2801. This show that the 12 point mutations introduced in the Bet v 1 (3007) mutant lowers the affinity for specific serum IgE.

References

1. WO 97/30150 (Pangenetics B. V., Molecules for the induction of immunological tolerance)

2. WO 92/02621 (Biomay Biotechnik Produktions—und Handelsgesellschaft mbH, Allergens of Alder pollen and applications thereof)

3. WO 90/11293 (Immunologic Pharmaceutical Corporation, The University of North Carolina at Chapel Hill, Allergenic proteins from ragweed and uses thereof)

4. Takai T, Yokota T, Yasue M, Nishiyama C, Yuuki T, Mori A, Okudaira H, Okumura Y: “Engineering of the major house dust mite allergen Der f 2 for allergen-specific immunotherapy”. Nat Biotechnol 15, 754-758 (1997).

5. Smith A M, Chapman M D: “Localization of antigenic sites on Der p 2 using oligonucleotide-directed mutagenesis targeted to predicted surface residues”. Clin Exp Allergy 27, 593-599 (1997).

6. Aki T, Ono K, Hidaka Y, Shimonishi Y, Jyo T, Wada T, Yamashita M, Shigeta S, Murooka Y, Oka S: “Structure of IgE epitopes on a new 39-kD allergen molecule from the house dust mite, Dermatophagoides farinae”. Int Arch Allergy Immunol 103, 357-364 (1994).

7. Forster E, Dudler T, Gmachl M, Aberer W, Urbanek R, Suter M: “Natural and recombinant enzymatically active or inactive bee venom phospholipase A2 has the same potency to release histamine from basophils in patients with Hymenoptera allergy”. J Allergy Clin Immunol 95,1229-1235 (1995).

8. Burks A W, Shin D, Cockrell G, Stanley J S, Helm R M, Bannon G A: “Mapping and mutational analysis of the IgE-binding epitopes on Ara h 1, a legume vicilin protein and a major allergen in peanut hypersensitivity”. Eur J Biochem 245, 334-339 (1997).

9. Stanley J S, King N, Burks A W, Huang S K, Sampson H, Cockrell G, Helm R M, West C M, Bannon G A: “Identification and mutational analysis of the immunodominant IgE binding epitopes of the major peanut allergen Ara h 2”. Arch Biochem Biophys 342, 244-253 (1997).

10. Ferreira F, Rohlfs A, Hoffmann-Sommergruber K, Schenk S, Ebner C, Briza P, Jilek A, Kraft D, Breitenbach M, Scheiner O: “Modulation of IgE-binding properties of tree pollen allergens by site-directed mutagenesis”. Adv Exp Med Biol 409, 127-135 (1996).

11. Ferreira F, Ebner C, Kramer B, Casari G, Briza P, Kungl A J, Grimm R, Jah-Schmid B, Breiteneder H, Kraft D, Breitenbach M, Rheinberger H -J, Scheiner O, “Modulation of IgE reactivity of allergens by site-directed mutagenesis: Potential use of hypeallergenic variants for immunotherapy”, FASEB Journal for Experimental Biology Vol. 12, No. 2, February 1998, 231-242 (1998).

12. Wiedemann P, Giehl K, Almo S C, Fedorov A A, Girvin M, Steinberger P, Rudiger M, Ortner M, Sippl M, Dolecek C, Kraft D, Jockusch B, Valenta R: “Molecular and structural analysis of a continuous birch profilin epitope defined by a monoclonal antibody”. J Biol Chem 271, 29915-29921 (1996).

13. Alvarez A M, Fukuhara E, Nakase M, Adachi T, Aoki N, Nakamura R, Matsuda T: “Four rice seed cDNA clones belonging to the alpha-amylase/trypsin inhibitor gene family encode potential rice allergens”. Biosci Biotechnol Biochem 59,1304-1308 (1995).

14. Colombo P, Kennedy D, Ramsdale T, Costa M A, Djro G, Izzo V, Salvadori S, Guerrini R, Cocchiara R, Mirisola M G, Wood S, Geraci D, Journal of Immunology Vol.160, No. 6, Mar. 15, 1998, 2780-2875.

15. Spangfort M D, Ipsen H, Sparholt S H, Aasmul-Olsen S, Larsen M R, Mortz E, Roepstorff P, Larsen J N: “Characterization of Purified Recombinant Bet v 1 with Authentic N-terminus, Cloned in Fusion with Maltose-Binding Protein”. Prot Exp Purification 8, 365-373 (1996a).

16. Ipsen H, Wihl J -Å, Petersen B N, Lwenstein H: “Specificity mapping of patients IgE response towards the tree pollen major allergens Aln g I, Bet v I and Cor a I.” Clin. Exp. Allergy 22, 391-9, (1992)

17. Gajhede M, Osmark P, Poulsen F M, Ipsen H, Larsen J N, Joost van Neerven R J, Schou C, Lwnstein H, and Spangfort M D: “X-ray and NMR structure of Bet v 1, the origin of birch pollen allergy”. Nature structural biology 3, 1040-1045 (1996).

18. Altschul S F, Gish W, Miller W, Myers E W, and Lipman D J: “Basic local alignment search tool”. J. Mol. Biol. 215, 403-410 (1990).

19. Higgins D, Thompson J, Gibson T, Thompson J D, Higgins D G, and Gibson T J: “CLUSTAL W: improving the sensitivity of progressive multiple sequence alignment through sequence weighting, position-specific gap penalties and weight matrix choice”. Nucleic Acids Res. 22, 4673-4680 (1994).

20. Saiki R K, Gelfand D H, Stoffel S, Scharf S J, Higuchi R, Horn G T, Mullis K B, Erlich H A: “Primer-directed enzymatic amplification of DNA with a thermostable DNA polymerase”. Science 239, 487-491 (1988).

21. Spangfort M D, Larsen J N, Gajhede M: “Crystallization and Preliminary X-ray Investigation at 2.0 Å Resolution of Bet v 1, a Birch Pollen Protein Causing IgE-Mediated Allergy”. PROTEINS, Struc Func Genet 26, 358-360 (1996b).

22. Monsalve R I, Lu G, and King T P: “Recombinant venom allergen, antigen 5 of yellowjacket (Vespula vulgaris) and paper wasp (Polistes annularis) by expression in bacteria or yeast” (1999) Submitted.

23. Fang K S F, Vitale M, Fehlner P and King T P: “cDNA cloning and primary structure of a white-face hornet venom allergen, antigen 5”. Proc. Natl. Acad. Sci. USA 85, 895 (1988).

24. Lu G, Villalba M, Coscia M R, Hoffman D R and King T P: “Sequence Analysis and Antigenic Cross-reactivity of a Venom Allergen, Antigen 5, from Hornets, Wasps, and Yellow Jackets”. Journal of Immunology 150, 2823-2830 (1993).

25. Punnonen J: “Molecular Breeding of Allergy Vaccines and Antiallergic Cytokines”. Int Arch Allergy Immunol 2000; 121:173-182.

26. P. A. Wurtzen, M. Wissenbach, H. Ipsen, A. Bufe, J. Arnved, and R. J. J. van Neerven. J Allergy Clin Immunol, 1999; 104: 115-23.

27. Sparholt S H, Larsen J N, Ipsen H, Schou C, van Neerven R J. Clin Exp Allergy Aug. 27, 1997;(8):932-41.

1. A recombinant Bet v 1 allergen, characterised in that it is a mutant of a naturally occurring Bet v 1 allergen wherein:
a. the mutant retains essentially the same α-carbon backbone structure as the naturally occurring allergen,
b. the mutant comprises at least four primary mutations, which each reduce the specific IgE binding capability of the mutated allergen as compared to the IgE binding capability of the naturally occurring Bet v 1 allergen,
c. each primary mutation is a substitution of one surface-exposed amino acid residue with another residue,
d. the mutations are placed in such a manner that at least one area of 400-800 Å² comprises either no mutations or one or more moderate mutations,
e. the primary mutations are selected from at least 4 of the following 10 groups, each group comprising surface exposed amino acid positions suitable for amino acid substitution:
group 1: A130, E131, K134, A135, K137, E138, E141, T142, R145;
group 2: V2, F3, N4, Y5, E6, T7, K119;
group 3: D27, S39, S40, Y41, E42, N43, I44, E45, G46, N47, P50, G51, K55, D72, E73;
group 4: E8, T10, V12, P14, V105, A106, T107, P108, D109, G 10, I113, K115;
group 5: A16, K20, S149, Y150, L152, A153, H154, S155, D156, Y158, N159, +160, wherein +160 represents addition of an N-terminal amino acid;
group 6: L24, D25, N28, K32;
group 7: H76, T77, N78, F79, K80, E101, K103;
group 8: K68, R70, I86, E87, E96, K97;
group 9: G1, G92, D93, T94, K123, G124, D125, H126, E127, K129;
group 10: P35, Q36, E60, G61, P63, F64, K65, Y66;
with the proviso that the recombinant Bet v 1 allergen is not one of the following specific mutants: (Asn28Thr, Lys32Gln, Asn78Lys, Lys103Val, Arg145Glu, Asp156His, +160Asn); (Tyr5Val, Glu42Ser, Glu45Ser, Asn78Lys, Lys103Val, Lys123Ile, Lys134Glu, Asp156His); (Tyr5Val, Glu45Ser, Lys65Asn, Lys97Ser, Lys134Glu); (Ala16Pro, Asn28Thr, Lys32Gln, Lys103Thr, Pro108Gly, Leu152Lys, Ala153Gly, Ser155Pro); (N28T, K32Q, N78K, K103V, P108G, R145E, D156H, +160N); (Tyr5Val, Lys134Glu, Asn28Thr, Lys32Gln, Glu45Ser, Lys65Asn, Asn78Lys, Lys103Val, Lys97Ser, Pro108Gly, Arg145Glu, Asp156His, +160Asn); (Tyr5Val, Lys134Glu, Glu42Ser, Glu45Ser, Asn78Lys, Lys103Val, Lys123Ile, Asp156His, +160Asn); (Asn28Thr, Lys32Gln, Lys65Asn, Glu96Leu, Lys97Ser, Pro108Gly, Asp109Asn, Asp125Tyr, Glu127Ser, Arg145Glu); (Y5V, N28T, K32Q, E42S, E45S, N78K, K103V, P108G, K123I, K134E, D156H, +160N); (Y5V, E42S, E45S, K65N, N78K, K97S, K103V, K123I, K134E, D156H, +160N); and (Y5V, N28T, K32Q, E42S, E45S, K65N, N78K, K97S, K103V, P108G, K123I, K134E, D156H, +160N).

2. A recombinant Bet v 1 allergen according to claim 1, wherein the primary mutations are selected from at least 4 of the following 10 groups, each group comprising surface exposed amino acid positions suitable for amino acid substitution:
group 1: A130, K134, A135, K137, E138, E141, T142, R145;
group 2: V2, F3, N4, Y5, E6, T7, K119;
group 3: D27, Y41, E42, N43, I44, E45, G46, N47, P50, G51, K55, D72, E73;
group 4: E8, T10, P108, D109, I113, K115;
group 5: H154, S155, D156, N159, +160;
group 6: D25, N28, K32;
group 7: H76, T77, N78, K80, E101, K103;
group 8: K68, R70, I86, E87, E96, K97;
group 9: G1, G92, T94, K123, G124, D125, H126;
group 10: K65, Y66.

3. A recombinant Bet v 1 allergen according to claim 2, wherein the primary mutations are selected from at least 4 of the following 10 groups, each group comprising surface exposed amino acid positions suitable for amino acid substitution:
group 1: A130, K134, A135, K137, E138, E141, T142;
group 2: V2, F3, N4, Y5, E6, T7, K119;
group 3: D27, Y41, N43, I44, E45, G46, N47, P50, G51, K55, D72, E73;
group 4: E8, P108, I113, K115;
group 5: H154, S155, N159, +160;
group 6: D25, N28;
group 7: H76, N78, K80, E101, K103;
group 8: K68, R70, I86, E87, E96, K97;
group 9: G1, G92, T94, G124, D125, H126;
group 10: Y66.

4. A recombinant Bet v 1 allergen according to claim 1, wherein the primary mutations are selected from at least 4 of the following 9 groups, each group comprising surface exposed amino acid positions suitable for amino acid substitution:
group 1: A130, A135, K137, E138, E141, T142;
group 2: F3, N4, E6, T7, K119;
group 3: D27, Y41, N43, I44, G46, P50, G51, D72, E73;
group 4: E8, I113, K115;
group 5: H154, S155, N159;
group 7: H76, N78, K80, E101;
group 8: K68, R70, I86, E87;
group 9: G1, G92, D93, G124, H126;
group 10: Y66.

5. A recombinant Bet v 1 allergen according to claim 1, where the primary mutations are selected from at least 4 of the following 10 groups, each group comprising surface exposed amino acid positions suitable for the following specific amino acid substitutions:
group 1: A130: A130V, A130G, A130I, A130L, A130S, A130H, A130T; E131: E131D, E131H, E131K, E131R, E131S; K134: K134R, K134H, K134S, K134Q, K1341, K134E; A135: A135V, A135G, A135I, A135L, A135S, A135H, A135T; K137: K137R, K137H, K137S, K137Q, K137I, K137E; E138: E138D, E138H, E138K, E138R, E138S, E138N; E141: E141D, E141H, E141K, E141R, E141S; T142: T142A, T142S, T142L, T142V, T142D, T142K, T142N; R145: R145K, R145H, R145T, R145D, R145E;
group 2: V2: V2A, V2I, V2K, V2L, V2R, V2T; F3: F3H, F3W, F3S, F3D; N4: N4H, N4K, N4M, N4Q, N4R; Y5: Y5D, Y5G, Y5H, Y5I, Y5K, Y5V; E6: E6D, E6H, E6K, E6R, E6S; T7: T7P, T7S, T7L, T7V, T7D, T7K, T7N; K119: K119R, K119H, K119S, K119Q, K119I, K119E, K119N;
group 3: D27: D27E, D27H, D27K, D27R, D27S; S39: S39T, S39L, S39V, S39D, S39K; S40: S40T, S40L, S40V, S40D, S40K; Y41: Y41D, Y41G, Y41H, Y41I, Y41K, Y41V; E42: E42S, E42D, E42H, E42K, E42R; N43: N43H, N43K, N43M, N43Q, N43R; I44:I44L, I44K, I44R, I44D; E45: E45S, E45D, E45H, E45K, E45R; G46: G46N, G46H, G46K, G46M, G46Q, G46R; N47: N47H, N47K, N47M, N47Q, N47R; P50: P50G; G51: G51N, G51H, G51K, G51M, G51Q, G51R; K55: K55R, K55H, K55S, K55Q, K55I, K55E, K55N; D72: D72E, D72S, D72H, D72R, D72K; E73: E73D, E73S, E73H, E73R, E73K;
group 4: E8: E8D, E8H, E8K, E8R, E8S; T10: T10P, T10S, T10L, T10V, T10D, T10K, T10N; V12: V12A, V12I, V12K, V12L, V12R, V12T; P14: P14G; V105: V105A, V105I, V105K, V105L, V105R, V105T; A106: A106V, A106G, A106I, A106L, A106S, A106H, A106T; T107: T107A, T107S, T107L, T107V, T107D, T107K, T107N; P108: P108G; D109: D109N D109E, D109S, D109H, D109R, D109K; G110: G110N, G110H, G110K, G110M, G110Q, G110R; I113: I113L, I113K, I113R, I113D, K115: K115R, K115H, K115S, K115Q, K115I, K115E, K115N;
group 5: A16: A16V, A16G, A16I, A16L, A16S, A16H, A16T; K20: K20R, K20H, K20S, K20Q, K20I, K20E, K20N; S149: S149T, S149L, S149V, S149D, S149K; Y150: Y150T, Y150L, Y150V, Y150D, Y150K; L152: L152A, L152V, L152G, L152I, L152S, L152H, L152T; A153: A153V, A153G, A1531, A153L, A153S, A153H, A153T; H154: H154W, H154F, H154S, H154D; S155: S155T, S155L, S155V, S155D, S155K; D156: D156H, D156E, D156S, D156R, D156K; Y158: Y158D, Y158G, Y158H, Y158I, Y158K, Y158V; N159: N159H, N159K, N159M, N159Q, N159R, N159G, +160N;
group 6: L24: L24A, L24V, L24G, L24I, L24S, L24H, L24T; D25: D25E, D25H, D25K, D25R, D25S; N28: N28H, N28K, N28M, N28Q, N28R, N28T; K32: K32Q, K32R, K32N, K32H, K32S, K32I, K32E;
group 7: H76: H76W, H76F, H76S, H76D; T77: T77A, T77S, T77L, T77V, T77D, T77K, T77N; N78: N78H, N78K, N78M, N78Q, N78R; F79: F79H, F79W, F79S, F79D; K80: K80R, K80H, K80S, K80Q, K80I, K80E, K80N; E101: E101D, E101H, E101K, E101R, E101S; K103: K103R, K103H, K103S, K103Q, K103I, K103E, K103V;
group 8: K68: K68R, K68H, K68S, K68Q, K68I, K68E, K68N; R70: R70K, R70H, R70T, R70D, R70E, R70N; I86: I86L, I86K, I86R, I86D; E87: E87D, E87H, E87K, E87R, E87S, E87A; E96: E96D, E96H, E96K, E96R, E96S, E96L; K97: K97R, K97H, K97S, K97Q, K97I, K97E;
group 9: G1: G1N, G1H, G1K, G1M, G1Q, G1R; G92: G92N, G92H, G92K, G92M, G92Q, G92R; D93: D93N, D93E, D93S, D93H, D93R, D93K; T94: T94A, T94S, T94L, T94V, T94D, T94K, T94N; K123: K123R, K123H, K123S, K123Q, K123I, K123E; G124: G124N, G124H, G124K, G124M, G124Q, G124R; D125: D125E, D125H, D125K, D125R, D125S, D125Y; H126: H126W, H126F, H126S, H126D; E127: E127D, E127H, E127K, E127R, E127S; K129: K129R, K129H, K129S, K129Q, K129I, K129E, K129N;
group 10: P35: P35G; Q36: Q36K, Q36R, Q36N, Q36H, Q36S, Q36I, Q36E; E60: E60H, E60K, E60M, E60Q, E60R; G61: G61N, G61H, G61K, G61M, G61Q, G61R; P63: P63G; F64: F64H, F64W, F64S, F64D; K65: K65R, K65H, K65S, K65Q, K651, K65E, K65N; Y66: Y66D, Y66G, Y66H, Y66I, Y66K, Y66V.

6. A recombinant Bet v 1 allergen according to claim 1, where the primary mutations are selected from at least 4 of the following 10 groups, each group comprising surface exposed amino acid positions suitable for the following specific amino acid substitutions:
group 1: A130: A130V, A130G, A130I, A130L, A130S, A130H, A130T; K134: K134R, K134H, K134S, K134Q, K134I, K134E; A135: A135V, A135G, A135I, A135L, A135S, A135H, A135T; K137: K137R, K137H, K137S, K137Q, K137I, K137E; E138: E138D, E138H, E138K, E138R, E138S, E138N; E141: E141D, E141H, E141K, E141R, E141S; T142: T142A, T142S, T142L, T142V, T142D, T142K, T142N; R145: R145K, R145H, R145T, R145D, R145E;
group 2: V2: V2A, V2I, V2K, V2L, V2R, V2T; F3: F3H, F3W, F3S, F3D; N4: N4H, N4K, N4M, N4Q, N4R; Y5: Y5D, Y5G, Y5H, Y5I, Y5K, Y5V; E6: E6D, E6H, E6K, E6R, E6S; T7: T7P, T7S, T7L, T7V, T7D, T7K, T7N; K119: K119R, K119H, K119S, K119Q, K119I, K119E, K119N;
group 3: D27: D27E, D27H, D27K, D27R, D27S; Y41: Y41D, Y41G, Y41H, Y41I, Y41K, Y41V; E42: E42S, E42D, E42H, E42K, E42R; N43: N43H, N43K, N43M, N43Q, N43R; I44: I44L, I44K, I44R, I44D; E45: E45S, E45D, E45H, E45K, E45R; G46: G46N, G46H, G46K, G46M, G46Q, G46R; N47: N47H, N47K, N47M, N47Q, N47R; P50: P50G; G51: G51N, G51H, G51K, G51M, G51Q, G51R; K55: K55R, K55H, K55S, K55Q, K55I, K55E, K55N; D72: D72E, D72S, D72H, D72R, D72K; E73: E73D, E73S, E73H, E73R, E73K;
group 4: E8: E8D, E8H, E8K, E8R, E8S; T10: T10P, T10S, T10L, T10V, T10D, T10K, T10N; P108: P108G; D109: D109N D109E, D109S, D109H, D109R, D109K; I113: I113L, I113K, I113R, I113D, K115: K115R, K115H, K115S, K115Q, K115I, K115E, K115N;
group 5: H154: H154W, H154F, H154S, H154D; S155: S155T, S155L, S155V, S155D, S155K; D156: D156H, D156E, D156S, D156R, D156K; N159: N159H, N159K, N159M, N159Q, N159R, N159G, +160N;
group 6: D25: D25E, D25H, D25K, D25R, D25S; N28: N28H, N28K, N28M, N28Q, N28R, N28T; K32: K32Q, K32R, K32N, K32H, K32S, K32I, K32E;
group 7: H76: H76W, H76F, H76S, H76D; T77: T77A, T77S, T77L, T77V, T77D, T77K, T77N; N78: N78H, N78K, N78M, N78Q, N78R; K80: K80R, K80H, K80S, K80Q, K80I, K80E, K80N; E101: E101D, E101H, E101K, E101R, E101S; K103: K103R, K103H, K103S, K103Q, K103I, K103E, K103V;
group 8: K68: K68R, K68H, K68S, K68Q, K68I, K68E, K68N; R70: R70K, R70H, R70T, R70D, R70E, R70N; I86: I86L, I86K, I86R, I86D; E87: E87D, E87H, E87K, E87R, E87S, E87A; E96: E96D, E96H, E96K, E96R, E96S, E96L; K97: K97R, K97H, K97S, K97Q, K971, K97E;
group 9: G1: G1N, G1H, G1K, G1M, G1Q, G1R; G92: G92N, G92H, G92K, G92M, G92Q, G92R; T94: T94A, T94S, T94L, T94V, T94D, T94K, T94N; K123: K123R, K123H, K123S, K123Q, K123I, K123E; G124: G124N, G124H, G124K, G124M, G124Q, G124R; D125: D125E, D125H, D125K, D125R, D125S, D125Y; H126: H126W, H126F, H126S, H126D;
group 10: K65: K65R, K65H, K65S, K65Q, K65I, K65E, K65N; Y66: Y66D, Y66G, Y66H, Y66I, Y66K, Y66V.

7. A recombinant Bet v 1 allergen according to claim 1, where the primary mutations are selected from at least 4 of the following 10 groups, each group comprising surface exposed amino acid positions suitable for the following specific amino acid substitutions:
group 1: A130: A130V, A130G, A130I, A130L, A130S, A130H, A130T; K134: K134R, K134H, K134S, K134Q, K134I, K134E; A135: A135V, A135G, A135I, A135L, A135S, A135H, A135T; K137: K137R, K137H, K137S, K137Q, K137I, K137E; E138: E138D, E138H, E138K, E138R, E138S, E138N; E141: E141D, E141H, E141K, E141R, E141S; T142: T142A, T142S, T142L, T142V, T142D, T142K, T142N;
group 2: V2: V2A, V2I, V2K, V2L, V2R, V2T; F3: F3H, F3W, F3S, F3D; N4: N4H, N4K, N4M, N4Q, N4R; Y5: Y5D, Y5G, Y5H, Y5I, Y5K, Y5V; E6: E6D, E6H, E6K, E6R, E6S; T7: T7P, T7S, T7L, T7V, T7D, T7K, T7N; K119: K119R, K119H, K119S, K119Q, K119I, K119E, K119N;
group 3: D27: D27E, D27H, D27K, D27R, D27S; Y41: Y41D, Y41G, Y41H, Y41I, Y41K, Y41V; N43: N43H, N43K, N43M, N43Q, N43R; I44: I44L, I44K, I44R, I44D; E45: E45S, E45D, E45H, E45K, E45R; G46: G46N, G46H, G46K, G46M, G46Q, G46R; N47: N47H, N47K, N47M, N47Q, N47R; P50: P50G; G51: G51N, G51H, G51K, G51M, G51Q, G51R; K55: K55R, K55H, K55S, K55Q, K551, K55E, K55N; D72: D72E, D72S, D72H, D72R, D72K; E73: E73D, E73S, E73H, E73R, E73K;
group 4: E8: E8D, E8H, E8K, E8R, E8S; P108: P108G; I113: I113L, I113K, I113R, I113D, K 15: K115R, K115H, K115S, K115Q, K115I, K115E, K115N;
group 5: H154: H154W, H154F, H154S, H154D; S155: S155T, S155L, S155V, S155D, S155K; N159: N159H, N159K, N159M, N159Q, N159R, N159G, +160N;
group 6: D25: D25E, D25H, D25K, D25R, D25S; N28: N28H, N28K, N28M, N28Q, N28R, N28T;
group 7: H76: H76W, H76F, H76S, H76D; N78: N78H, N78K, N78M, N78Q, N78R; K80: K80R, K80H, K80S, K80Q, K80I, K80E, K80N; E101: E101D, E101H, E101K, E101R, E101S; K103: K103R, K103H, K103S, K103Q, K103I, K103E, K103V;
group 8: K68: K68R, K68H, K68S, K68Q, K68I, K68E, K68N; R70: R70K, R70H, R70T, R70D, R70E, R70N; I86: I86L, I86K, I86R, I86D; E87: E87D, E87H, E87K, E87R, E87S, E87A; E96: E96D, E96H, E96K, E96R, E96S, E96L; K97: K97R, K97H, K97S, K97Q, K971, K97E;
group 9: G1: G1N, G1H, G1K, G1M, G1Q, G1R; G92: G92N, G92H, G92K, G92M, G92Q, G92R; T94: T94A, T94S, T94L, T94V, T94D, T94K, T94N; G124: G124N, G124H, G124K, G124M, G124Q, G124R; D125: D125E, D125H, D125K, D125R, D125S, D125Y; H126: H126W, H126F, H126S, H126D;
group 10: Y66: Y66D, Y66G, Y66H, Y66I, Y66K, Y66V.

8. A recombinant Bet v 1 allergen according to claim 1, where the primary mutations are selected from at least 4 of the following 8 groups, each group comprising surface exposed amino acid positions suitable for the following specific amino acid substitutions:
group 1: A130: A130V, A130G, A130I, A130L, A130S, A130H, A130T; A135: A135V, A135G, A135I, A135L, A135S, A135H, A135T; K137: K137R, K137H, K137S, K137Q, K137I, K137E; E138: E138D, E138H, E138K, E138R, E138S, E138N; E141: E141D, E141H, E141K, E141R, E141S; T142: T142A, T142S, T142L, T142V, T142D, T142K, T142N;
group 2: F3: F3H, F3W, F3S, F3D; N4: N4H, N4K, N4M, N4Q, N4R; E6: E6D, E6H, E6K, E6R, E6S; T7: T7P, T7S, T7L, T7V, T7D, T7K, T7N; K119: K119R, K119H, K119S, K119Q, K119I, K119E, K119N;
group 3: D27: D27E, D27H, D27K, D27R, D27S; Y41: Y41D, Y41G, Y41H, Y41I, Y41K, Y41V; N43: N43H, N43K, N43M, N43Q, N43R; I44: I44L, I44K, I44R, I44D; G46: G46N, G46H, G46K, G46M, G46Q, G46R; N47: N47H, N47K, N47M, N47Q, N47R; P50: P50G; G51: G51N, G51H, G51K, G51M, G51Q, G51R; D72: D72E, D72S, D72H, D72R, D72K; E73: E73D, E73S, E73H, E73R, E73K;
group 4: E8: E8D, E8H, E8K, E8R, E8S; I113: I113L, I113K, I113R, I113D, K115: K115R, K115H, K115S, K115Q, K115I, K115E, K115N;
group 5: H154: H154W, H154F, H154S, H154D; S155: S155T, S155L, S155V, S155D, S155K; N159: N159H, N159K, N159M, N159Q, N159R, N159G,+160N;
group 7: H76: H76W, H76F, H76S, H76D; N78: N78H, N78K, N78M, N78Q, N78R; K80: K80R, K80H, K80S, K80Q, K801, K80E, K80N; E101: E101D, E101H, E101K, E101R, E101S; group 8: K68: K68R, K68H, K68S, K68Q, K681, K68E, K68N; R70: R70K, R70H, R70T, R70D, R70E, R70N; I86: I86L, I86K, I86R, I86D; E87: E87D, E87H, E87K, E87R, E87S, E87A;
group 9: G1: G1N, G1H, G1K, G1M, G1Q, G1R; G92: G92N, G92H, G92K, G92M, G92Q, G92R; D93: D93N, D93E, D93S, D93H, D93R, D93K; G124: G124N, G124H, G124K, G124M, G124Q, G124R; H126: H126W, H126F, H126S, H126D;
group 10: Y66: Y66D, Y66G, Y66H, Y66I, Y66K, Y66V.

9. A recombinant Bet v 1 allergen according to claim 1 that comprises the following mutations: Y5V, E45S, N78K, K97S, K103V, K134E, +160N.

10. A recombinant Bet v 1 allergen according to claim 9 that further comprises at least one of the following substitutions: E8/K115, D125/H126, E138/K137/E141, D25/N28, E87/K55, S155/H154/N159, N47/P50/H76/N43/144/R70, E87/K55, E73/P50/D72, A130, N28/D25, P108, V2/K119/N4/E6/E96.

11. A recombinant Bet v 1 allergen according to claim 9 that further comprises at least one of the following substitutions: T10P, K65N, N28/D25/K32Q/E141/K137/E138, D125/K123I/H126, P108/D109N, E42S/K55/144/N43, E73/D72, E87, E96/K119, A130, V2/E6, E8/K115, N47/P50/R70/H76/T77A, S155/D156H/N159, E6N2.

12. A recombinant Bet v 1 allergen according to claim 10 that further comprises at least one of the following substitutions: T10P, K65N, N28/D25/K32Q/E141/K137/E138, D125/K123I/H126, P108/D109N, E42S/K55/144/N43, E73/D72, E87, E96/K119, A130, V2/E6, E8/K115, N47/P50/R70/H76/T77A, S155/D156H/N159, E6N2.

13. A recombinant Bet v 1 allergen according to claim 1 that comprises the following mutations: Y5V, N28T, K32Q, E45S, N78K, K97S, K103V, K134E, +160N.

14. A recombinant Bet v 1 allergen according to claim 13 that further comprises at least one of the following substitutions: E8/K115, D125/H126, E138/K137/E141, E87/K55, S155/H154/N159, N47/P50/H76/N43/144/R70, K55, E73/P50/D72, A130, D25, P108, V2/K 119/N4/E6/E96.

15. A recombinant Bet v 1 allergen according to claim 13 that further comprises at least one of the following substitutions: T10P, K65N, E141/K137/E138, D125/K123I/H126, P108/D109N, E42S/K55/144/N43, E73/D72, E87, V2/E6, N47/P50/R70/H76/T77A. E96/K119, A130, E8/K115, S155/D156H/H154/N159, E6N2.

16. A recombinant Bet v 1 allergen according to claim 14 that further comprises at least one of the following substitutions: T10P, K65N, E141/K137/E138, D125/K123I/H126, P108/D109N, E42S/K55/144/N43, E73/D72, E87, V2/E6, N47/P50/R70/H76/T77A. E96/K119, A130, E8/K115, S155/D156H/H154/N159, E6N2.

17. A recombinant Bet v 1 allergen according to claim 1 that comprises the following mutations: Y5V, N28T, K32Q, E45S, N78K, E87S, K97S, K103V, K134E, N159G, +160N.

18. A recombinant allergen according to claim 17, that further comprises at least one of the following substitutions: K55, A138/K137/E141, D125/H126, P108, V2/N4/K119/E6, S155/H154, N47/P50/H76, E73, R70, A130, E8/K115, E96.

19. A recombinant allergen according to claim 17, that further comprises at least one of the following substitutions: K65N, T10P, D125, K123I, P108, D109N, N47/P50/H76, E138/K137/El41, E42S/K55/144/N43, S155/D156H, E73/D72, E6N2, E96.

20. A recombinant allergen according to claim 18, that further comprises at least one of the following substitutions: K65N, T10P, D125, K123I, P108, D109N, N47/P50/H76, E138/K137/El41, E42S/K55/144/N43, S155/D156H, E73/D72, E6N2, E96.

21. A recombinant Bet v 1 allergen according to claim 1 that comprises the following mutations: Y5V, N28T, K32Q, E45S, N78K, K97S, K103V, P108G, D125Y, K134E, +160N.

22. A recombinant Bet v 1 allergen according to claim 21 that further comprises at least one of the following substitutions: E87, E141, K55, N47/N43/144/H76, S155/HIS154, A130, E8, E73, V2/K119.

23. A recombinant Bet v 1 allergen according to claim 21 that further comprises at least one of the following substitutions: K65N, T10P/E8, E87, S155/D156H, E141, E42S, A130, E8/T10P, N47, H76T, V2.

24. A recombinant Bet v 1 allergen according to claim 22 that further comprises at least one of the following substitutions: K65N, T10P/E8, E87, S155/D156H, E141, E42S, A130, E8/T10P, N47, H76T, V2.

25. A recombinant Bet v 1 allergen according to claim 1 that comprises the following mutations: Y5V, N28T, K32Q, E45S, E73S, E96S, P108G, D125Y, N159G, +160N.

26. A recombinant Bet v 1 allergen according to claim 25 that further comprises at least one of the following substitutions: K134, N78, E87, K119, E8, K55X, E141, N47, S155, E6, K103, A130, V2.

27. A recombinant Bet v 1 allergen according to claim 25 that further comprises at least one of the following substitutions: K65N/K55, T10P/E8/E141, E138/K134, E87, E42S/K55/144, S155/D156H, N78, K119N2/N4, N47/P50, H76/T77A, A130, E6/K115/K103.

28. A recombinant Bet v 1 allergen according to claim 26 that further comprises at least one of the following substitutions: K65N/K55, T10P/E8/E141, E138/K134, E87, E42S/K55/144, S155/D156H, N78, K119N21N4, N47/P50, H76/T77A, A130, E6/K115/K103.

29. A recombinant Bet v 1 allergen according to claim 1 that comprises the following mutations: Y5V, N28T, K32Q, E45S, E96S, P108G, +160N.

30. A recombinant Bet v 1 allergen according to claim 29 that further comprises at least one of the following substitutions: K134, N78, E87, K119, E8, K55X, E141, S155, N47, E6, K103, A130, V2, R70, D125.

31. A recombinant Bet v 1 allergen according to claim 29 that further comprises at least one of the following substitutions: N78/T77A, K103X, K134/E138, K65N/K55, T10P, D125/H126, E42S/K55, S155/D156H/HIS154, K119N2, E87, N47/P50/H76, A130.

32. A recombinant Bet v 1 allergen according to claim 30 that further comprises at least one of the following substitutions: N78/T77A, K103X, K134/E138, K65N/K55, T10P, D125/H126, E42S/K55, S155/D156H/HIS154, K119/V2, E87, N47/P50/H76, A130.

33. A recombinant Bet v 1 allergen according to claim 1 that comprises at least one of the following substitutions: Y5, N28, K32, E45, E96/K97, P108/D109, N159/+160, E60, T10, K103/K115, K65, K129, K134, E42/K55, S149/A153/L152, D125/K123, N47/L24, T77/N78, K119, E87, A16/K20/P14, Q36/G61/P63, E73, D93, V2.

34. A recombinant Bet v 1 allergen according to claim 1 that comprises at least one of the following substitutions: Y5V, N28T, K32Q, E45S, E96S/K97S, P108G/D109N, N159G/+160N, E60S, T10N, K103V/K115N, K129N, K134E, E42S/K55N, S149T/A153V/L152A, D125Y/K123I, N47K/L24A, T77N/N78K, K119N, E87A, A116G/K20S/P14G, Q36N/G61S/P63G, E73S, D93S, V2L.

35. A recombinant Bet v 1 mutant allergen according to claim 1 comprising substitutions that are selected from at least four of the following 10 groups:
group 1: A130V, K134E, E141N,
group 2: V2L, Y5V, E6S, K 119N,
group 3: E42S, E45S, N47K, K55N, E73S, E73T, E73S,
group 4: E8S, T10P, P14G, P108G, D109N, K115N,
group 5: A16G, K20S, S149T L152A A153V, S155T, N159G, +160N,
group 6: L24A, D25E, N28T, K32Q,
group 7: T77A, T77N, N78K, K103V,
group 8: R70N, E87A, E96S, K97S,
group 9: D93S, K1231, D125Y, K129N,
group 10: Q36N, E60S, G61S, P63G.

36. A recombinant Bet v 1 mutant allergen according to claim 1 comprising substitutions that are selected from at least four of the following 10 groups:
group 1: K134E,
group 2: Y5V, K119N, V2L,
group 3: E45S, E42S, K55N, N47K, E73S,
group 4: E96S, K97S, P108G, D109N, T10N, K115N, P14G,
group 5: N159G, +160N, S149T, A153V, L152A, A16G, K20S,
group 6: N28T, K32Q, L24A,
group 7: K103V, T77N, N78K,
group 8: E96S, K97S, E87A,
group 9: K129N, D125Y, K123I, D93S,
group 10: E60S, Q36N, G61S, P63G.

37. A recombinant Bet v 1 allergen according to claim 1 comprising 5-10 primary mutations.

38. A recombinant Bet v 1 allergen according claim 1 that further comprises at least one secondary mutation.

39. A recombinant Bet v 1 allergen according to claim 1 that further comprises at least one secondary mutation selected from the groups listed in claim 1.

40. A recombinant Bet v 1 allergen according to claim 1 that further comprises at least one additional mutation wherein the mutation is an addition or deletion of a surface exposed loop amino acid residue.

41. A composition comprising two or more recombinant mutant Bet v 1 allergen variants according to claim 1 wherein each variant is defined by having at least one primary mutation, which is absent in at least one of the other variants.

42. A composition according to claim 41 comprising 2-12 of said variants.

43. A composition according to claim 41 that is a pharmaceutical composition.

44. A composition according to claim 43 that is suitable for preventing and/or treating Fagales pollen allergy.

45. A composition according to claim 43 that is suitable for preventing and/or treating birch pollen allergy.

46. A pharmaceutical composition characterised in that it comprises a recombinant allergen according to claim 1, optionally in combination with a pharmaceutically acceptable carrier and/or excipient, and optionally an adjuvant.

47. A pharmaceutical composition characterised in that it comprises a composition according to claim 41, optionally in combination with a pharmaceutically acceptable carrier and/or excipient, and optionally an adjuvant.

48. A pharmaceutical composition according to claim 46, characterised in that it is in the form of a vaccine against allergic reactions elicited by a naturally occurring Bet v 1 allergen in patients suffering from birch pollen allergy.

49. A pharmaceutical composition according to claim 47, characterised in that it is in the form of a vaccine against allergic reactions elicited by a naturally occurring Bet v 1 allergen in patients suffering from birch pollen allergy.

50. A method of generating an immune response in a subject comprising administering to a subject a recombinant allergen according to claim 1.

51. A method of generating an immune response in a subject comprising administering to a subject a composition according to claim 41.

52. A method of generating an immune response in a subject comprising administering to a subject a pharmaceutical composition according to claim 45.

53. Vaccination or treatment of a subject comprising administering to the subject a recombinant allergen according to claim 1.

54. Vaccination or treatment of a subject comprising administering to the subject a composition according to claim 41.

55. Vaccination or treatment of a subject comprising administering to the subject a pharmaceutical composition according to claim 45.

56. A process for preparing a pharmaceutical composition according to claim 45 comprising mixing a recombinant allergen or a composition with pharmaceutically acceptable substances and/or excipients.

57. A pharmaceutical composition obtainable by the process according to claim 56.

58. A method for the treatment, prevention or alleviation of allergic reactions in a subject comprising administering to a subject a recombinant Bet v 1 allergen according to claim 1.

59. A method for the treatment, prevention or alleviation of allergic reactions in a subject comprising administering to a subject, a composition according to claim 41.

60. A method for the treatment, prevention or alleviation of allergic reactions in a subject comprising administering to a subject a pharmaceutical composition according to claim 57.

61. A method of preparing a recombinant Bet v 1 allergen according to claim 1 wherein the substitution of amino acids is carried out by site-directed mutagenesis.

62. A method of preparing a recombinant Bet v 1 allergen according to claim 1, wherein the allergen is produced by DNA shuffling (molecular breeding).

63. A method of preparing a recombinant Bet v 1 allergen library according to claim 1 wherein the allergen is produced by using oligonucleotide primers accommodating random substitutions of at least four amino residues.

64. A method according to claim 63 wherein the amino acid residues are selected from the group consisting of: Y5, T10, K20, N28, K32, Q36, E42, E45, E73, K65, N78, E87, K97, K103, P108, K123, K129, K134, S149, D156, and +160.

65. A DNA sequence encoding a recombinant Bet v 1 allergen according to claim 1, a derivative thereof, a partial sequence thereof, a degenerated sequence thereof or a sequence which hybridises thereto under stringent conditions, wherein said derivative, partial sequence, degenerated sequence or hybridising sequence encodes a peptide having at least one B cell epitope.

66. A DNA sequence according to claim 65, which is a derivative of the DNA sequence encoding the naturally occurring allergen.

67. A DNA sequence according to claim 66 wherein the derivative is obtained by site-directed mutagenesis of the DNA encoding the naturally occurring Bet v 1 allergen.

68. An expression vector comprising the DNA according to claim 65.

69. A host cell comprising the expression vector of claim 68.

70. A method of producing a recombinant mutant Bet v 1 allergen comprising the step of cultivating the host cell of claim 69.

71. A recombinant Bet v 1 allergen according to claim 1 comprising at least one T cell epitope capable of stimulating a T cell clone or T cell line specific for the naturally occurring Bet v 1 allergen.

72. A recombinant Bet v 1 allergen that is encoded by the DNA sequence according to claim 65 comprising at least one T cell epitope capable of stimulating a T cell clone or T cell line specific for the naturally occurring Bet v 1 allergen.

73. A diagnostic assay for assessing relevance, safety, or outcome of therapy of a subject using a recombinant mutant Bet v 1 allergen according to claim 1, wherein an IgE containing sample of a subject is mixed with said mutant and assessed for the level of reactivity between the IgE in said sample and said mutant.

74. A diagnostic assay for assessing relevance, safety, or outcome of therapy of a subject using a composition according to claim 41, wherein an IgE containing sample of a subject is mixed with mutant or composition and assessed for the level of reactivity between the IgE in said sample and said mutant.


SEQUENCE LISTING (89 sequences)

SEQ ID NO: 1 - protein, 160 aa, Betula verrucosa
MGVFNYETETTSVIPAARLFKAFILDGDNLFPKVAPQAISSVENIEGNGGPGTIKKISFPEGLPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 2 - protein, 160 aa, Betula verrucosa
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 3 - protein, 160 aa, Betula verrucosa
  MISC_FEATURE (1)..(160): Point mutated Bet v 1 allergen
GVFNVETETTSVIPAARLFKAFILDGDNLFPKVAPQAISSVSNIEGNGGPGTIKKISFPEGFPFKYVKDRVDEVDHTKFKYNYSVIEGGPIGDTLESISNEIVIVATPDGGSILKISNKYHTKGDHEVKAEQVEASKEMGETLLRAVESYLLAHSDAYNN

SEQ ID NO: 4 - protein, 160 aa, Betula verrucosa
  MISC_FEATURE (1)..(160): Point mutated Bet v 1 allergen
GVFNVETETTSVIPAARLFKAFILDGDTLFPQVAPQAISSVENISGNGGPGTIKKISFPEGFPFKYVKDRVDEVDHTKFKYNYSVIEGGPIGDTLESISNEIVIVATPDGGSILKISNKYHTKGDHEVKAEQVEASKEMGETLLRAVESYLLAHSDAYNN

SEQ ID NO: 5 - protein, 160 aa, Betula verrucosa
  MISC_FEATURE (1)..(160): Point mutated Bet v 1 allergen
GVFNVETETTSVIPAARLFKAFILDGDTLFPQVAPQAISSVENISGNGGPGTIKKISFPEGFPFKYVKDRVDEVDHTKFKYNYSVIEGGPIGDTLESISNEIVIVATGDGGSILKISNKYHTKGYHEVKAEQVEASKEMGETLLRAVESYLLAHSDAYNN

SEQ ID NO: 6 - protein, 160 aa, Betula verrucosa
  MISC_FEATURE (1)..(160): Point mutated Bet v 1 allergen
GVFNVETETTSVIPAARLFKAFILDGDTLFPQVAPQAISSVENISGNGGPGTIKKISFPEGFPFKYVKDRVDEVDHTNFKYNYSVIEGGPIGDTLSKISNEIKIVATGDGGSILKISNKYHTKGDHEVKAEQVKASKEMGETLLRAVESYLLAHSDAYNN

SEQ ID NO: 7 - protein, 160 aa, Betula verrucosa
  MISC_FEATURE (1)..(160): Point mutated Bet v 1 allergen
GVFNVETETTSVIPAARLFKAFILDGDTLFPQVAPQAISSVENISGNGGPGTIKKISFPEGFPFKYVKDRVDEVDHTKFKYNYSVIEGGPIGDTLESISNEIVIVATPDGGSILKISNKYHTKGDHEVKAEQVEASKEMGETLLRAVESYLLAHSDAYGN

SEQ ID NO: 8 - protein, 160 aa, Betula verrucosa
  MISC_FEATURE (1)..(160): Point mutated Bet v 1 allergen
GVFNVETETTSVIPAARLFKAFILDGDTLFPKVAPQAISSVENISGNGGPGTIKKISFPEGFPFKYVKDRVDSVDHTNFKYNYSVIEGGPIGDTLSKISNEIKIVATGDGGSILKISNKYHTKGYHEVKAEQVKASKEMGETLLRAVESYLLAHSDAYGN

SEQ ID NO: 9 - protein, 160 aa, Betula verrucosa
  MISC_FEATURE (1)..(160): Point mutated Bet v 1 allergen
GVFNVETSTTSVIPAARLFKAFILTGDTLFPQVAPQAISSVENISGKGGPGTIKNISFPEGLPFKYVKDRVDEVDHTKFKYNYSVIAGGPIGDTLESISNEIVIVATPDGGSILKISNKYHTKGYHEVKAEQVEASKEMGSTLLRAVESYLLAHTDAYNN

SEQ ID NO: 10 - protein, 160 aa, Betula verrucosa
  MISC_FEATURE (1)..(160): Point mutated Bet v 1 allergen
GLFNVETSTTSVIPAARLFKAFILTGDTLFPQVAPQAISSVENISGKGGPGTIKNISFPEGLPFKYVKDRVDTVDHTKFKYNYSVIAGGPIGDTLESISNEIVIVATGDGGSILKISNKYHTKGYHEVKVEQVEASKEMGSTLLRAVESYLLAHTDAYNN

SEQ ID NO: 11 - protein, 160 aa, Betula verrucosa
  MISC_FEATURE (1)..(160): Point mutated Bet v 1 allergen
GLFNVETETPSVIPAARLFKAFILDGDTLFPQVAPQAISSVSNISGKGGPGTIKKISFPEGLPFNYVKDRVDTVDHTTFKYNYSVIAGGPIGDTLESISNEIVIVATPNGGSILKISNKYHTIGDHEVKAEQVEASKEMGNTLLRAVESYLLAHSDAYNN

SEQ ID NO: 12 - protein, 160 aa, Betula verrucosa
  MISC_FEATURE (1)..(160): Point mutated Bet v 1 allergen
GLFNVSTSTPSVIPAARLFKAFILDGDTLFPQVAPQAISSVSNISGKGGPGTIKKISFPEGLPFNYVKDRVDTVDHTTFKYNYSVIAGGPIGDTLESISNEIVIVATPNGGSILKISNKYHTIGDHEVKAEQVEASKEMGNTLLRAVESYLLAHSHAYNN

SEQ ID NO: 13 - protein, 160 aa, Betula verrucosa
  MISC_FEATURE (1)..(160): Point mutated Bet v 1 allergen
GVFNVETETTSVIPAARLFKAFILDGDTLFPQVAPQAISSVENISGKGGPGTIKNISFPEGLPFKYVKDRVDEVDHTKFKYNYSVIAGGPIGDTLESISNEIVIVATGDGGSILKISNKYHTKGYHEVKAEQVEASKEMGNTLLRAVESYLLAHTDAYNN

SEQ ID NO: 14 - protein, 160 aa, Betula verrucosa
  MISC_FEATURE (1)..(160): Point mutated Bet v 1 allergen
GLFNVETSTTSVIPAARLFKAFILDGDTLFPQVAPQAISSVENISGKGGPGTIKNISFPEGLPFKYVKDRVDTVDHTKFKYNYSVIAGGPIGDTLESISNEIVIVATGDGGSILKISNKYHTKGYHEVKVEQVEASKEMGNTLLRAVESYLLAHTDAYNN

SEQ ID NO: 15 - protein, 160 aa, Betula verrucosa
  MISC_FEATURE (1)..(160): Point mutated Bet v 1 allergen
GVFNVETETPSVIPAARLFKAFILDGDTLFPQVAPQAISSVSNISGNGGPGTIKKISFPEGLPFNYVKDRVDEVDHTKFKYNYSVIAGGPIGDTLESISNEIVIVATGDGGSILKISNKYHTKGYHEVKAEQVEASKEMGNTLLRAVESYLLAHSHAYNN

SEQ ID NO: 16 - protein, 160 aa, Betula verrucosa
  MISC_FEATURE (1)..(160): Point mutated Bet v 1 allergen
GLFNVETSTPSVIPAARLFKAFILDGDTLFPQVAPQAISSVSNISGKGGPGTIKKISFPEGLPFNYVKDRVDEVDTTKFKYNYSVIAGGPIGDTLESISNEIVIVATGDGGSILKISNKYHTKGYHEVKVEQVEASKEMGNTLLRAVESYLLAHSHAYNN

SEQ ID NO: 17 - protein, 160 aa, Betula verrucosa
  MISC_FEATURE (1)..(160): Point mutated Bet v 1 allergen
GVFNVETSTTSVIPAARLFKAFILDGDTLFPQVAPQAISSVENISGKGGPGTIKNISFPEGLPFKYVKDRVDSVDHTKFKYNYSVIAGGPIGDTLSKISNEIKIVATGDGGSILKISNNYHTKGYHEVKAEQVEASKEMGNTLLRAVESYLLAHSDAYGN

SEQ ID NO: 18 - protein, 160 aa, Betula verrucosa
  MISC_FEATURE (1)..(160): Point mutated Bet v 1 allergen
GLFNVSTSTTSVIPAARLFKAFILDGDTLFPQVAPQAISSVENISGKGGPGTIKNISFPEGLPFKYVKDRVDSVDHTKFKYNYSVIAGGPIGDTLSKISNEIVIVATGDGGSILKISNNYHTKGYHEVKVEQVEASKEMGNTLLRAVESYLLAHTDAYGN

SEQ ID NO: 19 - protein, 160 aa, Betula verrucosa
  MISC_FEATURE (1)..(160): Point mutated Bet v 1 allergen
GVFNVETETPSVIPAARLFKAFILDGDTLFPQVAPQAISSVSNISGNGGPGTIKKISFPEGLPFNYVKDRVDSVDHTKFKYNYSVIAGGPIGDTLSKISNEIKIVATGDGGSILKISNKYHTKGYHEVKAEQVKASKNMGETLLRAVESYLLAHSHAYGN

SEQ ID NO: 20 - protein, 160 aa, Betula verrucosa
  MISC_FEATURE (1)..(160): Point mutated Bet v 1 allergen
GLFNVSTETPSVIPAARLFKAFILDGDTLFPQVAPQAISSVSNISGNGGPGTIKKISFPEGLPFNYVKDRVDSVDHTKFKYNYSVIAGGPIGDTLSKISNEIVIVATGDGGSILKISNKYHTKGYHEVKVEQVKASKNMGETLLRAVESYLLAHTHAYGN

SEQ ID NO: 21 - protein, 160 aa, Betula verrucosa
  MISC_FEATURE (1)..(160): Point mutated Bet v 1 allergen
GVFNVETSTTSVIPAARLFKAFILDGDTLFPQVAPQAISSVENISGKGGPGTIKNISFPEGLPFKYVKDRVDEVDHTKFKYNYSVIAGGPIGDTLSKISNEIKIVATGDGGSILKISNNYHTKGDHEVKAEQVEASKNMGNTLLRAVESYLLAHTDAYNN

SEQ ID NO: 22 - protein, 160 aa, Betula verrucosa
  MISC_FEATURE (1)..(160): Point mutated Bet v 1 allergen
GLFNVSTSTTSVIPAARLFKAFILDGDTLFPQVAPQAISSVENISGKGGPGTIKNISFPEGLPFKYVKDNVDEVDHTKFKYNYSVIAGGPIGDTLSKISNEIVIVATGDGGSILKISNNYHTKGYHEVKVEQVEASKNMGNTLLRAVESYLLAHTDAYNN

SEQ ID NO: 23 - protein, 160 aa, Betula verrucosa
  MISC_FEATURE (1)..(160): Point mutated Bet v 1 allergen
GVFNVETETPSVIPAARLFKAFILDGDTLFPQVAPQAISSVSNISGNGGPGTIKKISFPEGLPFNYVKDRVDEVDHANFKYNYSVIEGGPIGDTLSKISNEIVIVATGDGGSILKISNKYHTKGYHEVKAEQVKASKNMGETLLRAVESYLLAHSDAYNN

SEQ ID NO: 24 - protein, 160 aa, Betula verrucosa
  MISC_FEATURE (1)..(160): Point mutated Bet v 1 allergen
GVFNVETETPSVIPAARLFKAFILDGDTLFPQVAPQAISSVSNISGNGGPGTIKKISFPEGLPFNYVKDRVDEVDHANFKYNYSVIAGGPIGDTLSKISNEIVIVATGDGGSILKISNNYHTKGYHEVKVEQVKASKNMGETLLRAVESYLLAHSHAYNN

SEQ ID NO: 25 - protein, 160 aa, Betula verrucosa
  MISC_FEATURE (1)..(160): Point mutated Bet v 1 allergen
GVFNVETETASVIPAARLFLAFILDGDTLFPQVAPPAISSVSNISGNGGPGTIKKISFPEGLPFNYVKDRVDPVDHTKFKYNYSVIGGGPIGDTLESISNEIVIVATPDGGSILKISNKYHTIGDHEVEAEQVEASKEMGETLLRAVEGYLLAHSHAYNN

SEQ ID NO: 26 - protein, 160 aa, Betula verrucosa; identical to SEQ ID NO: 25
  MISC_FEATURE (1)..(160): Point mutated Bet v 1 allergen

SEQ ID NO: 27 - protein, 160 aa, Betula verrucosa
  MISC_FEATURE (1)..(160): Point mutated Bet v 1 allergen
GVFNVETETPSVIPAARLFHAFILDGDTLFPQVAPKAISSVSNISGNGGPGTIKKISFPEGLPFNYVKDRVDRVDHTKFKYNYSVIEGGPIGDTLESISNEIVIVATPDGGSILKISNKYHTIGDHEVEAEQVEASKEMGETLLRAVEGYLLAHSHAYNN

SEQ ID NO: 28 - protein, 160 aa, Betula verrucosa
  MISC_FEATURE (1)..(160): Point mutated Bet v 1 allergen
GVFNVETETTSVIPAARLFHAFILDGDNLFPKVAPPAISSVSNISGNGGPGTIKKISFPEGLPFNYVKDRVDSVDHTKFKYNYSVIGGGPIGDTLESISNEIVIVATPDGGSILKISNKYHTIGDHEVEAEQVEASKEMGETLLRAVERYLLAHSHAYNN

SEQ ID NO: 29 - protein, 160 aa, Betula verrucosa
  MISC_FEATURE (1)..(160): Point mutated Bet v 1 allergen
GVFNVETETPSVIPAARLFMAFILDGDTLFPQVAPPAISSVSNISGNGGPGTIKKISFPEGLPFNYVKDRVDSVDHTNFKYNYSVIGGGPIGDTLESISNEIVIVATPDGGSILKISNKYHTIGDHEVEAEQVEASKEMGETLLRAVEAYLLAHSHAYNN

SEQ ID NO: 30 - protein, 160 aa, Betula verrucosa
  MISC_FEATURE (1)..(160): Point mutated Bet v 1 allergen
GVFNVETETPSVIPAARLFLAFILDGDNLFPKVAPPAISSVSNISGNGGPGTIKKISFPEGLPFNYVKDRVDTVDHTKFKYNYSVIGGGPIGDTLESISNEIVIVATPDGGSILKISNKYHTIGDHEVEAEQVEASKEMGETLLRAVERYLLAHSHAYNN

SEQ ID NO: 31 - protein, 160 aa, Betula verrucosa
  MISC_FEATURE (1)..(160): Point mutated Bet v 1 allergen
GVFNVETETPSVIPAARLFQAFILDGDNLFPKVAPPAISSVSNISGNGGPGTIKKISFPEGLPFNYVKDRVDSVDHTNFKYNYSVIGGGPIGDTLESISNEIVIVATPDGGSILKISNKYHTIGDHEVEAEQVEASKEMGETLLRAVEPYLLAHSHAYNN

SEQ ID NO: 32 - protein, 160 aa, Betula verrucosa
  MISC_FEATURE (1)..(160): Point mutated Bet v 1 allergen
GVFNVETETASVIPAARLFLAFILDGDNLFPKVAPPAISSVSNISGNGGPGTIKKISFPEGLPFNYVKDRVDGVDHTKFKYNYSVIDGGPIGDTLESISNEIVIVATPDGGSILKISNKYHTIGDHEVEAEQVEASKEMGETLLRAVERYLLAHSHAYNN

SEQ ID NO: 33 - protein, 160 aa, Betula verrucosa
  MISC_FEATURE (1)..(160): Point mutated Bet v 1 allergen
GVFNVETETASVIPAARLFLAFILDGDTLFPQVAPEAISSVSNISGNGGPGTIKKISFPEGLPFNYVKDRVDGVDHTNFKYNYSVIGGGPIGDTLESISNEIVIVATPDGGSILKISNKYHTIGDHEVEAEQVEASKEMGETLLRAVEAYLLAHSHAYNN

SEQ ID NO: 34 - protein, 160 aa, Betula verrucosa
  MISC_FEATURE (1)..(160): Point mutated Bet v 1 allergen
GVFNVETETPSVIPAARLFKAFILDGDNLFPKVAPPAISSVSNISGNGGPGTIKKISFPEGLPFNYVKDRVDSVDHTKFKYNYSVIGGGPIGDTLESISNEIVIVATPDGGSILKISNKYHTIGDHEVEAEQVEASKEMGETLLRAVETYLLAHSHAYNN

SEQ ID NO: 35 - protein, 160 aa, Betula verrucosa
  MISC_FEATURE (1)..(160): Point mutated Bet v 1 allergen
GVFNVETETPSVIPAARLFKAFILDGDNLFPKVAPPAISSVSNISGNGGPGTIKKISFPEGLPFNYVKDRVDRVDHTKFKYNYSVIGGGPIGDTLESISNEIVIVATPDGGSILKISNKYHTIGDHEVEAEQVEASKEMGETLLRAVERYLLAHSHAYNN

SEQ ID NO: 36 - protein, 160 aa, Betula verrucosa; identical to SEQ ID NO: 35
  MISC_FEATURE (1)..(160): Point mutated Bet v 1 allergen

SEQ ID NO: 37 - protein, 160 aa, Betula verrucosa
  MISC_FEATURE (1)..(160): Point mutated Bet v 1 allergen
GVFNVETETPSVIPAARLFLAFILDGDTLFPQVAPKAISSVSNISGNGGPGTIKKISFPEGLPFNYVKDRVDRVDHTKFKYNYSVIDGGPIGDTLESISNEIVIVATPDGGSILKISNKYHTIGDHEVEAEQVEASKEMGETLLRAVEPYLLAHSHAYNN

SEQ ID NO: 38 - protein, 160 aa, Betula verrucosa
  MISC_FEATURE (1)..(160): Point mutated Bet v 1 allergen
GVFNVETETPSVIPAARLFLAFILDGDTLFPQVAPKAISSVSNISGNGGPGTIKKISFPEGLPFNYVKDRVDGVDHTKFKYNYSVIGGGPIGDTLESISNEIVIVATPDGGSILKISNKYHTIGDHEVEAEQVEASKEMGETLLRAVETYLLAHSHAYNN

SEQ ID NO: 39 - protein, 160 aa, Betula verrucosa
  MISC_FEATURE (1)..(160): Point mutated Bet v 1 allergen
GVFNVETETPSVIPAARLFMAFILDGDNLFPKVAPPAISSVSNISGNGGPGTIKKISFPEGLPFNYVKDRVDGVDHTKFKYNYSVIDGGPIGDTLESISNEIVIVATPDGGSILKISNKYHTIGDHEVEAEQVEASKEMGETLLRAVEGYLLAHSHAYNN

SEQ ID NO: 40 - protein, 160 aa, Betula verrucosa
  MISC_FEATURE (1)..(160): Point mutated Bet v 1 allergen
GVFNVETETNSVIPAARLFKAFILDGDTLFPQVAPQAISSVSNISGNGGPGTIKKISFPSGLPFNYVKDRVDEVDHTNFKYNYSVIEGGPIGDTLESISNEIVIVATGDGGSILKISNKYHTKGYHEVNAEQVKASKEMGETLLRAVETYLLAHSDAYNN

SEQ ID NO: 41 - protein, 160 aa, Betula verrucosa
  MISC_FEATURE (1)..(160): Point mutated Bet v 1 allergen
GLFNVETETNSVIPAARLFKAFILDGDTLFPQVAPQAISSVSNISGKGGPGTIKKISFPSGLPFNYVKDRVDEVDHNNFKYNYSVIEGGPIGDTLESISNEIVIVATGDGGSILKISNKYHTKGYHEVNAEQVEASKEMGETLLRAVETYLLAHSDAYNN

SEQ ID NO: 42 - protein, 160 aa, Betula verrucosa
  MISC_FEATURE (1)..(160): Point mutated Bet v 1 allergen
GLFNVETETNSVIPAGRLFKAFILDGDTLFPQVAPNAISSVSNISGKGGPGTIKKISFPSGLPFNYVKDRVDSVDHNNFKYNYSVIAGGPIGSTLESISNEIVIVATGDGGSILKISNKYHTKGYHEVNAEQVEASKEMGETLLRAVETYLLAHSDAYNN

SEQ ID NO: 43 - protein, 161 aa, Betula verrucosa
  MISC_FEATURE (1)..(161): Point mutated Bet v 1 allergen
GVFNVETETTSVIPAARLFKAFILDGDTLFPQVAPQAISSVENISGNGGPGTIKKISFPEXGFPFKYVKDRVDEVDHTKFKYNYSVIEGGPIGDTLESISNEIVIVATGDGGSILKISNKYHTKGYHEVKAEQVEASKEMGETLLRAVESYLLAHSDAYNN

SEQ ID NO: 44 - protein, 161 aa, Betula verrucosa
  MISC_FEATURE (1)..(161): Point mutated Bet v 1 allergen
GVFNVETETTSVIPAARLFKAFILDGDTLFPQVAPQAISSVENISGNGGPGTIKKISFPEGFPFKYVKDRVDEVDHTKFKYNYSVIEGGPIGDXTLESISNEIVIVATGDGGSILKISNKYHTKGYHEVKAEQVEASKEMGETLLRAVESYLLAHSDAYNN

SEQ ID NO: 45 - protein, 161 aa, Betula verrucosa
  MISC_FEATURE (1)..(161): Point mutated Bet v 1 allergen
GVFNVETETTSVXIPAARLFKAFILDGDTLFPQVAPQAISSVENISGNGGPGTIKKISFPEGFPFKYVKDRVDEVDHTKFKYNYSVIEGGPIGDTLESISNEIVIVATGDGGSILKISNKYHTKGYHEVKAEQVEASKEMGETLLRAVESYLLAHSDAYNN

SEQ ID NO: 46 - protein, 162 aa, Betula verrucosa
  MISC_FEATURE (1)..(162): Point mutated Bet v 1 allergen
GVFNVETETTSVIPAARLFKAFILDGDTLFPQVAPQAISSVENISGNGGPGTIKKIXSFPEXGFPFKYVKDRVDEVDHTKFKYNYSVIEGGPIGDTLESISNEIVIVATGDGGSILKISNKYHTKGYHEVKAEQVEASKEMGETLLRAVESYLLAHSDAYNN

SEQ ID NO: 47 - protein, 159 aa, Betula verrucosa
  MISC_FEATURE (1)..(159): Point mutated Bet v 1 allergen (amino acid deletion of residue G111)
GVFNVETETTSVIPAARLFKAFILDGDTLFPQVAPQAISSVENISGNGGPGTIKKISFPEGFPFKYVKDRVDEVDHTKFKYNYSVIEGGPIGDTLESISNEIVIVATGDGSILKISNKYHTKGYHEVKAEQVEASKEMGETLLRAVESYLLAHSDAYNN

SEQ ID NO: 48 - DNA, 24 bp, Betula verrucosa
cagactaattcgagctcggtaccc

SEQ ID NO: 49 - DNA, 24 bp, Betula verrucosa
gaaggctttcctttcaaatacgtg

SEQ ID NO: 50 - DNA, 24 bp, Betula verrucosa
tttgaaagggaggccttcgggaaa

SEQ ID NO: 51 - DNA, 24 bp, Betula verrucosa
gacacattgctgaagatctccaac

SEQ ID NO: 52 - DNA, 24 bp, Betual verrucosa
ggagatcttcagcaatgtgtcgcc

SEQ ID NO: 53 - DNA, 24 bp, Betula verrucosa
gtggcaaccccggatggaggatcc

SEQ ID NO: 54 - DNA, 24 bp, Betula verrucosa
tcctccatccggggttgccactat

SEQ ID NO: 55 - DNA, 24 bp, Betula verrucosa
accaaaggttatcatgaggtgaag

SEQ ID NO: 56 - DNA, 24 bp, Betula verrucosa
cacctcatgataacctttggtgtg

SEQ ID NO: 57 - DNA, 38 bp, Betula verrucosa
gccggaattcattagttgttgtaggcatcggagtgtgc

SEQ ID NO: 58 - DNA, 24 bp, Betula verrucosa primer
actgagaccvcstctgttatccca

SEQ ID NO: 59 - DNA, 24 bp, Betula verrucosa
gataacagasgbggtctcagtctc

SEQ ID NO: 60 - DNA, 24 bp, Betula verrucosa
cgactgttcmwkgcctttatcctt

SEQ ID NO: 61 - DNA, 24 bp, Betula verrucosa
gataaaggcmwkgaacagtcgagc

SEQ ID NO: 62 - DNA, 24 bp, Betula verrucosa
gttgcacccvmrgccattagcagt

SEQ ID NO: 63 - DNA, 24 bp, Betula verrucosa
gctaatggcykbgggtgcaacctt

SEQ ID NO: 64 - DNA, 24 bp, Betula verrucosa
agcgtgatcgrwggcggtcccata

SEQ ID NO: 65 - DNA, 24 bp, Betula verrucosa
gggaccgccwycgatcacgctgta

SEQ ID NO: 66 - DNA, 27 bp, Betula verrucosa
attggtgaccatgaggtgrawgcagag

SEQ ID NO: 67 - DNA, 27 bp, Betula verrucosa
wtycacctcatggtcaccaatggtgtg

SEQ ID NO: 68 - DNA, 24 bp, Betula verrucosa
gccgttgagvsytacctcttggca

SEQ ID NO: 69 - DNA, 24 bp, Betula verrucosa
caagaggtarsbctcaacggccct

SEQ ID NO: 70 - DNA, 24 bp, Betula verrucosa
agagttgatvsygtggaccacaca

SEQ ID NO: 71 - DNA, 24 bp, Betula verrucosa
gtggtccacrsbatcaactctgtc

SEQ ID NO: 72 - DNA, 30 bp, Betula verrucosa
cgtcccaagctttcattagttgttgtaggc

SEQ ID NO: 73 - DNA, 38 bp, Betula verrucosa
cggaattcataatggattataaagatggtgtgtttaat

SEQ ID NO: 74 - DNA, 24 bp, Betula verrucosa
cagactaattcgagctcggtaccc

SEQ ID NO: 75 - DNA, 23 bp, Betula verrucosa
tttcctgaaatgttttcaacact

SEQ ID NO: 76 - DNA, 23 bp, Betula verrucosa
aacatttcaggaaatggagggcc

SEQ ID NO: 77 - DNA, 24 bp, Betula verrucosa
cacgtagttgaaagggaggccttc

SEQ ID NO: 78 - DNA, 24 bp, Betula verrucosa
tttcaactacgtgaaggacagagt

SEQ ID NO: 79 - DNA, 24 bp, Betula verrucosa
tttcaactacgtgaaggacagagt

SEQ ID NO: 80 - DNA, 24 bp, Betula verrucosa
tttcaactacgtgaaggacagagt

SEQ ID NO: 81 - DNA, 24 bp, Betula verrucosa
ggagagcatctccaacgagataaa

SEQ ID NO: 82 - DNA, 24 bp, Betula verrucosa
caggttgaagcaagtaaagaaatg

SEQ ID NO: 83 - DNA, 24 bp, Betula verrucosa
gcaggtcgactctagaggatccat

SEQ ID NO: 84 - DNA, 24 bp, Betula verrucosa
cagactaattcgacgtcggtaccc

SEQ ID NO: 85 - DNA, 24 bp, Betula verrucosa
cagtcgcggtgctgggataacaga

SEQ ID NO: 86 - DNA, 24 bp, Betula verrucosa
ccagcaccgcgactgttcaaggcc

SEQ ID NO: 87 - DNA, 24 bp, Betula verrucosa
cactatggttatctcgttggagat

SEQ ID NO: 88 - DNA, 24 bp, Betula verrucosa
gagataaccatagtggcaactggt

SEQ ID NO: 89 - DNA, 45 bp, Betula verrucosa
ttactgaattcattagttgtaggcatccgggtggcctttgaggta